(19) United States
(12) Patent Application Publication
(10) Pub. No.: US 2024/0215556 A1
(43) Pub. Date: Jul. 4, 2024

(54) TRANSGENIC RODENTS EXPRESSING CHIMERIC EQUINE-RODENT ANTIBODIES AND METHODS OF USE THEREOF

(71) Applicant: TRIANNI, INC., Wilmington, DE (US)

(72) Inventors: Werner MUELLER, Köln (DE); Peter BURROWS, Birmingham, AL (US); Bao DUONG, Pacifica, CA (US); Matthias WABL, San Francisco, CA (US)

(21) Appl. No.: 18/557,414

(22) PCT Filed: May 4, 2022
(86) PCT No.: PCT/US2022/027622

§ 371 Date: Oct. 26, 2023

Related U.S. Application Data
(60) Provisional application No. 63/184,440, filed on May 5, 2021.

(51) Int. Cl. A01K 67/0275; C07K 16/46; C12N 15/85

(52) U.S. Cl. CPC A01K 67/0275; C07K 16/462; C12N 15/8509; A01K 2217/072; A01K 2227/105; A01K 2267/01; C07K 2317/24

(57) ABSTRACT

Described herein are transgenic mammals that express immunoglobulins with equine variable domains, including transgenic rodents that express immunoglobulins with equine variable domains for the development of therapeutic equine antibodies.

### FIELD OF THE INVENTION

This invention relates to production of immunoglobulin molecules, including methods for generating transgenic mammals capable of producing antigen-specific antibody-secreting cells for the generation of equine monoclonal antibodies.

### BACKGROUND OF THE INVENTION

In the following discussion certain articles and methods are described for background and introductory purposes. Nothing contained herein is to be construed as an “admission” of prior art. Applicant expressly reserves the right to demonstrate, where appropriate, that the articles and methods referenced herein do not constitute prior art under the applicable statutory provisions.

Antibodies have emerged as important biological pharmaceuticals because they (i) exhibit exquisite binding properties that can target antigens of diverse molecular forms, (ii) are physiological molecules with desirable pharmacokinetics that make them well tolerated in treated humans and animals, and (iii) are associated with powerful immunological properties that naturally ward off infectious agents. Furthermore, established technologies exist for the rapid isolation of antibodies from laboratory animals, which can readily mount a specific antibody response against virtually any foreign substance not present natively in the body.

In their most elemental form, antibodies include two identical heavy (H) chains that are each paired with an identical light (L) chain. The N-termini of both H and L chains include a variable domain (V_{H} and V_{L}, respectively) that together provide the paired H-L chains with a unique antigen-binding specificity.

The exons that encode the antibody V_{H} and V_{L} domains do not exist in the germline DNA. Instead, each V_{H} exon is generated by recombination of randomly selected V_{H}, D, and J_{H} gene segments present in the immunoglobulin H chain locus; likewise, individual V_{L} exons are produced by the chromosomal rearrangements of randomly selected V_{L} and J_{L} gene segments in a light chain locus.

In mammals, the genome typically contains two alleles that can express the H chain, two alleles that can express the kappa (κ) L chain, and two alleles that can express the lambda (2) L chain (one allele from each parent). There are multiple V_{H}, D, and J_{H} gene segments at the immunoglobulin H chain locus as well as multiple V_{L} and J_{L} gene segments at both the immunoglobulin κ (IGK) and immunoglobulin λ (IGL) L chain loci (Collins and Watson (2018) Immunoglobulin Light Chain Gene Rearrangements, Receptor Editing and the Development of a Self-Tolerant Antibody Repertoire. Front. Immunol. 9:2249. (doi: 10.3389/fimmu.2018.02249)).

In the heavy chain locus, exons for the expression of different antibody classes (isotypes) also exist. For example, in equine animals, the encoded isotypes are IgM, IgD, IgG1, IgG2, IgG3, IgG4, IgG5, IgG6, IgG7, IgE, and IgA. Polymorphic variants (referred to as allotypes) also exist among the encoded isotypes and can be useful as allelic markers. In equine animals, polymorphic variants exist for IgM, IgG3, IgG4, IgG7, and IgE allotypes.

During B cell development, gene rearrangements occur first on one of the two homologous chromosomes that contain the H chain variable gene segments. In pre-B cells, the resultant V_{H} exon is then spliced at the RNA level to the Cu exons for IgM H chain (μH chain) expression. Most of the μH chain synthesized by pre-B cells is retained in the endoplasmic reticulum (ER) and eventually degraded due to the non-covalent interaction between the partially unfolded CHI domain of the μH chain and the resident ER chaperone BiP (Haas and Wabl, Nature, 306:387-9, 1983; Bole et al., J Cell Biol. 102:1558, 1986). However, a small fraction of the μH chains associate with a surrogate light chain complex, which includes invariant 25 and VpreB proteins. This association displaces BiP and allows the μH chain/\5/VpreB complex, together with an Iga/ß signaling molecule heterodimer, to exit the ER as a preB Cell Receptor (preBCR) and traffic through the secretory pathway to the plasma membrane.

Subsequently, V_{L}-J_{L} rearrangements occur on one L chain allele at a time until a functional L chain is produced, after which the L chain polypeptides can associate with the IgM H chain homodimers to form a fully functional antigen-specific B cell receptor (BCR), which is expressed on the surface of the immature B cell.

The immature B cells migrate to secondary lymphoid organs where they differentiate into mature B cells that can respond to cognate antigen and differentiate into antibody-secreting plasmacytes and memory B cells. With the assistance of T cells, the B cells can undergo isotype switching, which changes the antibody isotype from IgM to IgG, IgA or IgE, as well as somatic hypermutation, which can change the amino acid sequence of the V_{H} and V_{L} exons. Although these mutations are introduced randomly into the V_{H} and V_{L} exons, B cells with higher affinity for the immunizing antigen are able to take up more of the antigen, process it and present it to T follicular helper cells and thus are preferentially activated compared to B cells with low or no affinity for the immunizing antigen. As a result, the somatic mutations become enriched in the complementarity determining regions (CDR) 1, 2 and 3, because these are the regions of the V_{H} and V_{L} domains that interact with the antigen.

The genes encoding various mouse immunoglobulins have been extensively characterized. For example, Blankenstein and Krawinkel described the mouse variable heavy chain region in Eur. J. Immunol., 17:1351-1357 (1987). The equine immunoglobulin genes (e.g., from the thoroughbred horse, Equus caballus, Twilight breed) have been structurally characterized. Sun et al. (Dev. Comp. Immunol. 34:109 (2010)) and Talmadge et al. (Dev. Comp. Immunol. 1:33 (2013); Dev. Comp. Immunol. 46:171 (2014)) describe the Ig heavy and lambda light chain genes in the horse genome and Walther, et al., describe the molecular characterization of all the Ig loci (Igh, Igk and IgÀ) (Dev. Comp. Immunol. 3:303 (2015)).

The generation of transgenic animals-such as mice having varied immunoglobulin loci—has allowed the use of such transgenic animals in various research and development applications, e.g., in drug discovery and basic research into various biological systems. For example, the generation of transgenic mice bearing human immunoglobulin genes is described in International Application Nos. WO 90/10077 and WO 90/04036. WO 90/04036 describes a transgenic mouse with an integrated human immunoglobulin “mini” locus. WO 90/10077 describes a vector containing the immunoglobulin dominant control region for use in generating transgenic animals.

Numerous methods have been developed for modifying the mouse endogenous immunoglobulin variable region gene locus with, e.g., human immunoglobulin sequences, to create partly or fully human antibodies for drug discovery purposes. Examples of such mice include those described in, e.g., U.S. Pat. Nos. 7,145,056; 7,064,244; 7,041,871; 6,673,986; 6,596,541; 6,570,061; 6,162,963; 6,130,364; 6,091,001; 6,023,010; 5,593,598; 5,877,397; 5,874,299; 5,814,318; 5,789,650; 5,661,016; 5,612,205; and 5,591,669.

The use of antibodies that function as drugs is not limited to the prevention or therapy of human disease. Domestic animals such as horses suffer from afflictions similar to those of humans, e.g., cancer, atopic dermatitis and chronic pain. Monoclonal antibodies targeting CD20, IgE and Nerve Growth Factor, respectively, are already in veterinary use for treatment of some of these conditions. However, before clinical use, the monoclonal antibodies, which were made in mice, had to be equineized, i.e., their amino acid sequence had to be changed from mouse to horse to prevent an adverse immune response in the recipient horses.

Based on the foregoing, it is clear that a need exists for efficient and cost-effective methods to produce equine antibodies for the treatment of diseases in horses. More particularly, there is a need in the art for small, rapidly breeding, non-equine mammals capable of producing antigen-specific equine immunoglobulins, particularly for generating hybridomas capable of large-scale production of equine monoclonal antibodies. As such, there remains a need for improved methods for generating transgenic nonhuman animals that are capable of producing equine antibodies, for example, antibodies with equine V regions.

### SUMMARY OF THE INVENTION

This Summary is provided to introduce a selection of concepts in a simplified form that are further described below in the Detailed Description. This Summary is not intended to identify key or essential features of the claimed subject matter, nor is it intended to be used to limit the scope of the claimed subject matter. Other features, details, utilities, and advantages of the claimed subject matter will be apparent from the following written Detailed Description including those aspects illustrated in the accompanying drawings and defined in the appended claims.

Described herein are methods for producing mouse antibodies with equine immunoglobulin variable regions. In one aspect, an antibody with equine variable regions is provided that can be produced in a transgenic mammal or in an in vitro cell culture.

In one aspect, a non-equine mammalian cell or a non-equine mammal is provided that has a genome that includes a heterologous partly equine immunoglobulin locus. In one aspect, the heterologous locus includes coding sequences of the equine immunoglobulin variable region genes and non-coding sequences based on the endogenous immunoglobulin variable region locus of the non-equine mammalian host. In one aspect, the non-equine mammalian cell or mammal is capable of expressing a chimeric B cell receptor (BCR) or antibody that includes equine heavy (H) and light (L) chain variable regions and constant regions that are endogenous to the non-equine mammalian host cell or mammal. In one aspect, the transgenic mammalian host cell or mammal has a genome in which part or all of the endogenous immunoglobulin variable region gene locus has been removed.

To produce chimeric equine monoclonal antibodies in a non-equine mammalian host, the host genome should have at least one locus that expresses chimeric equine immunoglobulin H or L chain. In one aspect, the host genome includes one heavy chain locus and two light chain loci that express chimeric equine immunoglobulin H and L chains, respectively.

In some aspects, the partly equine immunoglobulin locus includes equine V_{H} coding sequences and non-coding sequences present in the endogenous V_{H} gene locus of the non-equine mammalian host. In some aspects, the partly equine immunoglobulin locus includes equine V_{H} coding sequences and non-coding regulatory or scaffold sequences present in the endogenous V_{H} gene locus of the non-equine mammalian host. In one aspect, the partly equine immunoglobulin locus includes equine D_{H} and J_{H} gene segment coding sequences and non-coding sequences present in the endogenous D_{H} and J_{H} gene segments of the non-equine mammalian host cell genome. In one aspect, the partly equine immunoglobulin locus includes equine D_{H} and J_{H} gene segment coding sequences and non-coding regulatory or scaffold sequences present in the endogenous D_{H} and J_{H} gene segments of the non-equine mammalian host cell genome.

In other aspects, the partly equine immunoglobulin locus includes equine V_{L} coding sequences and non-coding sequences present in the endogenous V_{L} gene locus of the non-equine mammalian host. In other aspects, the partly equine immunoglobulin locus includes equine V_{L} coding sequences and non-coding regulatory or scaffold sequences present in the endogenous V_{L} gene locus of the non-equine mammalian host. In one aspect, the heterologous partly equine immunoglobulin locus includes equine V_{L} coding sequences and equine J_{L} gene segment coding sequences and non-coding sequences present in the endogenous J_{L} gene segments of the non-equine mammalian host cell genome. In one aspect, the heterologous partly equine immunoglobulin locus includes equine V_{L} coding sequences and equine J_{L} gene segment coding sequences and non-coding regulatory or scaffold sequences present in the endogenous J_{L} gene segments of the non-equine mammalian host cell genome.

In one aspect, the non-equine mammal is a rodent, for example, a mouse or rat.

In one aspect, a method is provided for generating a non-equine mammalian cell that includes a partly equine immunoglobulin locus. In one aspect, the method includes: a) introducing two or more recombinase targeting sites into the genome of a non-equine mammalian host cell and integrating at least one site upstream and at least one site downstream of a genomic region that includes endogenous immunoglobulin V_{H}, D_{H} and J_{H} genes or endogenous V_{L} and J_{L} genes; and b) introducing into the non-equine mammalian host cell via recombinase-mediated cassette exchange (RMCE) a heterologous partly equine immunoglobulin variable gene locus that includes equine V_{H}, D_{H} and J_{H} gene or equine V_{L} and J_{L} gene coding sequences and non-coding sequences based on the non-coding sequences present in the endogenous immunoglobulin variable region gene locus of the non-equine mammalian host.

In another aspect, the method includes deleting the endogenous immunoglobulin variable region in the genome of the host animal that is flanked by the two heterologous recombinase-targeting sites prior to introducing into the non-equine mammalian host cell via RMCE a heterologous partly equine immunoglobulin variable gene locus.

In one aspect, the heterologous partly equine immunoglobulin locus includes equine V_{H} gene segment coding sequences, equine D_{H} and J_{H} gene segment coding sequences and non-coding regulatory or scaffold sequences upstream of the equine D_{H} gene segments (Pre-D sequences, FIG. 1) based on the sequences present upstream of the endogenous D_{H} gene segments in the genome of the non-equine mammalian host. In one aspect, the upstream scaffold sequences contain non-immunoglobulin genes, such as Adam6 (FIG. 1), which is related to male fertility (Nishimura et al. Developmental Biol. 233(1): 204-213 (2011)). In one aspect, the partly equine immunoglobulin locus is introduced into the host cell using recombinase targeting sites that were previously introduced upstream of the endogenous immunoglobulin V_{H} gene locus and downstream of the endogenous J_{H} gene locus on the same chromosome.

In one aspect, the scaffold sequences includes a naturally occurring nucleic acid sequence from another species. In one aspect, the scaffolding sequences can be designed based on a naturally occurring nucleic acid sequence from another species, for example, the scaffolding sequences can include a naturally occurring nucleic acid sequence from another species that has been modified, for example, by one or more nucleic acid substitutions, insertions, deletions or other modifications. In one aspect, the scaffolding sequences can include an artificial sequence. In one aspect, the scaffold sequence includes sequences that are present in the immunoglobulin locus of the equine genome in combination with other sequences, for example, scaffold sequences from other species.

In another aspect, the heterologous partly equine immunoglobulin locus includes equine immunoglobulin V_{L} gene segment coding sequences, equine J_{L} gene segment coding sequences and non-coding sequences based on the non-coding sequences present in the endogenous L chain locus of the non-equine mammalian host cell genome. In one aspect, the non-coding sequences includes regulatory or scaffold sequences. In one aspect, the heterologous partly equine immunoglobulin locus is introduced into the host cell using recombinase targeting sites that have been previously introduced upstream of the endogenous immunoglobulin V_{L} gene locus and downstream of the endogenous J_{L} gene locus on the same chromosome.

In one aspect, the heterologous partly equine immunoglobulin locus is synthesized as a single nucleic acid and introduced into the non-equine mammalian host cell as a single nucleic acid region. The heterologous partly equine immunoglobulin locus may also be synthesized in two or more contiguous segments and introduced to the mammalian host cell as discrete segments. The heterologous partly equine immunoglobulin locus can also be produced using recombinant methods and isolated prior to being introduced into the non-equine mammalian host cell. In one aspect, a partly equine immunoglobulin heavy chain variable region locus can be generated in silico as follows: the genomic sequence of a mouse heavy chain immunoglobulin locus is obtained as well as equine V_{H}, D and J_{H} coding sequences, for example, from the National Center for Biotechnology Information. The mouse V_{H}, D and J_{H} coding sequences are replaced in silico with equine V_{H}, D and J_{H} coding sequences, for example, using commercially available software. Advantageously, the V_{H}, D and J_{H} coding sequences can be replaced while leaving the intervening mouse non-coding sequences intact. Similarly, a partly equine immunoglobulin light chain variable region locus can be generated in silico as follows: the genomic sequence of a mouse light chain immunoglobulin locus is obtained as well as equine V_{L} and J_{L} coding sequences, for example, from the National Center for Biotechnology Information. The mouse V_{L} and J_{L} coding sequences are replaced in silico with equine V_{L} and J_{L} coding sequences, for example, using commercially available software. Again, the V_{L} and J_{L} coding sequences can be replaced while leaving the intervening mouse non-coding sequences intact. Methods are known for synthesizing a DNA sequence that includes the partly equine immunoglobulin locus based on the in silico sequences.

In another aspect, a method is provided for generating a non-equine mammalian cell that includes a heterologous partly equine immunoglobulin locus. In one aspect, the method includes: a) introducing into the genome of a non-equine mammalian host cell two or more sequence-specific recombination sites that are not capable of recombining with one another, wherein at least one recombination site is introduced upstream of an endogenous immunoglobulin variable region gene locus and at least one recombination site is introduced downstream of the same endogenous immunoglobulin variable region gene locus; b) providing a vector that includes a heterologous partly equine immunoglobulin locus having i) equine immunoglobulin variable region gene coding sequences and ii) non-coding regulatory or scaffold sequences based on an endogenous immunoglobulin variable region gene locus of the host cell genome, wherein the partly equine immunoglobulin locus is flanked by the same two sequence-specific recombination sites that flank the endogenous immunoglobulin variable region gene locus of the host cell; c) introducing into the host cell the vector of step b) and a site specific recombinase capable of recognizing the two recombinase sites; d) allowing a recombination event to occur between the genome of the cell and the heterologous partly equine immunoglobulin locus, resulting in a replacement of the endogenous immunoglobulin variable region gene locus with the heterologous partly equine immunoglobulin variable region gene locus. In one aspect, the partly equine immunoglobulin locus includes equine V_{H} immunoglobulin gene segment coding sequences, and i) equine D_{H} and J_{H} gene segment coding sequences, ii) non-coding regulatory or scaffold sequences flanking individual V_{H}, D_{H}, and J_{H} gene segments present endogenously in the genome of the non-equine mammalian host, and iii) pre-D sequences based on the endogenous genome of the non-equine mammalian host cell. In one aspect, the recombinase targeting sites are introduced upstream of the endogenous immunoglobulin V_{H} gene locus and downstream of the endogenous J_{H} gene loci.

In one aspect, a transgenic rodent is provided with a genome in which a rodent endogenous immunoglobulin variable gene locus has been deleted and replaced with a heterologous partly equine immunoglobulin locus that includes equine immunoglobulin variable gene coding sequences and non-coding regulatory or scaffold sequences based on the rodent endogenous immunoglobulin variable gene locus. In one aspect, the heterologous partly equine immunoglobulin locus of the transgenic rodent is functional and expresses immunoglobulin chains that include equine variable domains and rodent constant domains. In one aspect, the heterologous partly equine immunoglobulin locus includes equine V_{H}, D_{H}, and J_{H} coding sequences. In one aspect, the heterologous partly equine immunoglobulin locus includes equine V_{L} and J_{L} coding sequences. In one aspect, the heterologous partly equine immunoglobulin locus includes equine kappa (κ) V_{L} and J_{L} coding sequences. In one aspect, the heterologous partly equine immunoglobulin locus includes equine lambda (2) V_{L} and J_{L} coding sequences. In one aspect, a cell of B lymphocyte lineage from the transgenic rodent is provided. In one aspect, a part or whole immunoglobulin molecule that includes equine variable domain and rodent constant domain sequences obtained from the cell of B lymphocyte lineage are provided. In one aspect, a hybridoma cell derived from the cell of B lymphocyte lineage is provided. In one aspect, a part or whole immunoglobulin molecule that includes equine variable domains and rodent constant domains derived from the hybridoma cell is provided. In one aspect, an immortalized cell derived from the cell of B lymphocyte lineage is provided. In one aspect, a part or whole immunoglobulin molecule that includes equine variable domains and rodent constant domains derived from an immortalized cell is provided. In one aspect, a transgenic rodent is provided, wherein the heterologous partly equine immunoglobulin locus includes equine V_{L} and J_{L} coding sequences. In one aspect, a transgenic rodent is provided, in which the heterologous partly equine immunoglobulin loci includes equine V_{H}, D_{H}, and J_{H} coding sequences. In one aspect, the heterologous partly equine immunoglobulin locus includes equine kappa (κ) V_{L} and J_{L} coding sequences. In one aspect, the heterologous partly equine immunoglobulin locus includes equine lambda (2) V_{L} and J_{L} coding sequences. In one aspect, the rodent is a mouse. In one aspect, the non-coding regulatory sequences include the one or more of the following sequences of the endogenous host: promoters preceding each V gene segment, splice sites, and recombination signal sequences for V(D)J recombination. In one aspect, the heterologous partly equine immunoglobulin locus further includes one or more of the following sequences of the endogenous host: ADAM6 gene, a Pax-5-Activated Intergenic Repeat (PAIR) elements, and CTCF binding sites from heavy chain intergenic control region 1 (IGCR1).

In one aspect, the non-equine mammalian cell is a mammalian cell. In one aspect, the non-equine mammalian cell is a mammalian embryonic stem (ES) cell.

In one aspect, non-equine mammalian cells in which the endogenous immunoglobulin variable region gene locus has been replaced with a heterologous partly equine immunoglobulin variable region gene locus are selected and isolated. In one aspect, the cells are non-equine mammalian ES cells, for example, rodent ES cells. In one aspect, at least one isolated non-equine mammalian cell is used to create a transgenic non-equine mammal expressing the heterologous partly equine immunoglobulin variable region gene loci. In one aspect, at least one isolated non-equine mammalian ES cell is used to create a transgenic non-equine mammal expressing the heterologous partly equine immunoglobulin variable region gene loci.

In one aspect, a method for generating the transgenic rodent is provided. In one aspect, the method includes: a) integrating at least one target site for a site-specific recombinase into the genome of a rodent cell upstream of an endogenous immunoglobulin variable gene locus and at least one target site for a site-specific recombinase downstream of the endogenous immunoglobulin variable gene locus. In one aspect, the endogenous immunoglobulin variable locus includes V_{H}, D_{H} and J_{H} gene segments. In one aspect, the endogenous immunoglobulin variable locus includes Vκ and Jκ gene segments. In one aspect, the endogenous immunoglobulin variable locus includes VA and JA gene segments. In one aspect, the endogenous immunoglobulin variable locus includes VA, JA gene segments and Cλ genes. In one aspect, the method includes: b) providing a vector that includes an heterologous partly equine immunoglobulin locus. In one aspect, said heterologous partly equine immunoglobulin locus includes chimeric equine immunoglobulin gene segments. In one aspect, each of the partly equine immunoglobulin gene segments include equine immunoglobulin variable gene coding sequences and rodent non-coding regulatory or scaffold sequences. In one aspect, the partly equine immunoglobulin variable gene locus is flanked by target sites for a site-specific recombinase. In one aspect, the target sites are capable of recombining with target sites introduced into the rodent cell. In one aspect, the method includes: c) introducing into the rodent cell the vector and a site-specific recombinase capable of recognizing the target sites. In one aspect, the method includes: d) allowing a recombination event to occur between the genome of the cell and the heterologous partly equine immunoglobulin locus, wherein the endogenous immunoglobulin variable gene locus is replaced with the heterologous partly equine immunoglobulin locus. In one aspect, the method includes: e) selecting a cell that includes the heterologous partly equine immunoglobulin variable locus generated in step d); and using the cell to create a transgenic rodent that includes the heterologous partly equine immunoglobulin variable locus. In one aspect, the cell is a rodent embryonic stem (ES) cell. In one aspect, the cell is a mouse embryonic stem (ES) cell.

In one aspect, the method further includes after step a) and before step b), a step of deleting the endogenous immunoglobulin variable gene locus by introducing a recombinase that recognizes a first set of target sites, wherein the deleting step leaves in place at least one set of target sites that are not capable of recombining with one another in the genome of the rodent cell. In one aspect, the vector includes equine V_{H}, D_{H}, and J_{H}, coding sequences. In one aspect, the vector includes equine V_{L} and J_{L} coding sequences. In one aspect, the heterologous partly equine immunoglobulin locus includes equine kappa (κ) V_{L} and J_{L} coding sequences. In one aspect, the heterologous partly equine immunoglobulin locus includes lambda (λ) V_{L} and J_{L} coding sequences. In one aspect, the vector further includes one or more of the following: a promoter, splice sites, and recombination signal sequences.

In one aspect, a method is provided for generating a transgenic non-equine mammal that includes a heterologous partly equine immunoglobulin variable region gene locus. In one aspect, the method includes: a) introducing into the genome of a non-equine mammalian host cell one or more sequence-specific recombination sites that flank an endogenous immunoglobulin variable region gene locus and are not capable of recombining with one another. In one aspect, the method includes: b) providing a vector that includes a partly equine immunoglobulin locus having i) equine variable region gene coding sequences and ii) non-coding regulatory or scaffold sequences based on the endogenous host immunoglobulin variable region gene locus. In one aspect, the coding and non-coding regulatory or scaffold sequences are flanked by the same sequence-specific recombination sites as those introduced to the genome of the host cell of a). In one aspect, the method includes: c) introducing into the cell the vector of step b) and a site-specific recombinase capable of recognizing one set of recombinase sites. In one aspect, the method includes: d) allowing a recombination event to occur between the genome of the cell of a) and the heterologous partly equine immunoglobulin variable region gene locus. In one aspect, the endogenous immunoglobulin variable region gene locus is replaced with the partly equine immunoglobulin locus. In one aspect, the method includes: e) selecting a cell that includes the partly equine immunoglobulin locus; and f) using the cell to create a transgenic mammal that includes the partly equine immunoglobulin locus.

In one aspect, the transgenic non-equine mammal is a rodent, e.g., a mouse or a rat.

In one aspect, an immunoglobulin library (also referred to as repertoire) is provided that includes a diversity of at least 10³ library members.

In one aspect, a repertoire of antibodies is provided that includes the partly equine antibody described herein. In one aspect, the repertoire includes a diversity of antibodies, that each specifically recognize the same target antigen. Such repertoire can be referred to as an antibody library of the same antibody type or structure, wherein antibodies differ in their antigen-binding sites, e.g., to produce antibody variants of a parent antibody recognizing the same epitope. In one aspect, the antibody library includes affinity matured or otherwise optimized antibody variants. In one aspect, the antibody library includes antibodies that specifically recognize a target antigen, but different epitopes of such target antigen.

In one aspect, the antibody repertoire is screened and individual library members are selected according to desired structural or functional properties, for example, to produce an antibody product.

In one aspect, a repertoire of antibodies is provided that include the partly equine antibody described herein. In one aspect, the repertoire includes a diversity of antibodies that recognize different target antigens. In one aspect, the repertoire is obtained by immunizing the non-equine mammal with multicomponent antigens, including, but not limited to, as viruses or bacteria, which can have many different target antigens, each of which can include multiple epitopes.

In one aspect, the repertoire is a naïve library of antibodies, which can also be referred to as a “pre-immune repertoire”. In one aspect, the pre-immune repertoire is expressed by mature but antigen-inexperienced B cells that have recently exited from the bone marrow.

In one aspect, the repertoire of antibodies can be characterized by a diversity encompassing at least about 103 antibodies, for example, at least about 10+, about 105, about 106 or about 107, each characterized by a different antigen-binding site.

In one aspect, a non-equine mammalian cell is provided that expresses a heterologous immunoglobulin variable region gene locus having equine variable region gene coding sequences and non-coding regulatory or scaffold sequences based on the endogenous non-equine immunoglobulin locus of the host genome. In one aspect, the non-equine mammalian cell expresses chimeric antibodies that include fully equine H or L chain variable domains in conjunction with their respective constant regions that are endogenous to the non-equine mammalian cell or mammal.

In one aspect, a non-equine transgenic mammal is provided that expresses a heterologous immunoglobulin variable region gene locus having equine variable region gene coding sequences and non-coding regulatory or scaffold sequences based on the endogenous non-equine immunoglobulin locus of the host genome. In one aspect, the non-equine transgenic mammal expresses chimeric antibodies that include fully equine H or L chain variable domains in conjunction with their respective constant regions that are endogenous to the non-equine mammalian cell or mammal.

In one aspect, B cells from transgenic non-equine mammals are provided that are capable of expressing partly equine antibodies having fully equine variable sequences. In one aspect, immortalized B cells are provided as a source of a monoclonal antibody specific for a particular antigen.

In one aspect, equine immunoglobulin variable region gene sequences are provided that are cloned from B cells for use in the production or optimization of antibodies for diagnostic, preventative and therapeutic uses.

In one aspect, non-equine hybridoma cells are provided that are capable of producing partly equine monoclonal antibodies having fully equine immunoglobulin variable region sequences.

In one aspect methods are provided for removing V_{H} and V_{L} exons that encode H and L chain immunoglobulin variable domains from monoclonal antibody-producing hybridomas and modifying the V_{H} and V_{L} exons to include equine constant regions, thereby creating a fully equine antibody that is not immunogenic when injected into horses.

In one aspect, a method of producing an equine antibody for therapeutic or diagnostic use is provided. In one aspect, the method includes:
(i) expressing an antibody with an equine variable domain cloned from an antibody-producing cell of a transgenic rodent whose genome includes an endogenous rodent immunoglobulin locus variable region that has been deleted and replaced with an heterologous immunoglobulin locus variable region that includes at least one of each of a chimeric V_{H}, D_{H} and J_{H} immunoglobulin variable region gene segment at the immunoglobulin heavy chain locus, and/or at least one of each of a chimeric V_{L} and J_{L} variable gene segment at the immunoglobulin light chain loci, wherein each chimeric gene segment that includes equine V, D or J immunoglobulin variable region coding sequences and rodent immunoglobulin variable region non-coding gene segment sequences; and
(ii) isolating the antibody with the equine variable domain, wherein the antibody is suitable for therapeutic or diagnostic use.

In one aspect, the antibody is cloned from a B cell of the transgenic rodent. In one aspect, the rodent is a mouse. In one aspect, a therapeutic or diagnostic antibody is provided that is produced by a method described herein.

In one aspect, a method of producing a therapeutic or diagnostic antibody with equine variable domains is provided. In one aspect, the method includes:
(i) cloning an equine variable domain of an antibody expressed by an antibody-producing cell from a transgenic rodent whose genome includes an endogenous rodent immunoglobulin locus variable region that has been deleted and replaced with an heterologous immunoglobulin locus variable region includes at least one of each of a chimeric V_{H}, D_{H} and J_{H} immunoglobulin variable region gene segment at the immunoglobulin heavy chain locus, and/or at least one of each of a chimeric V_{L} and J_{L} variable gene segment at the immunoglobulin light chain loci, wherein each chimeric gene segment includes equine V, D or J immunoglobulin variable region coding sequences and rodent immunoglobulin variable region non-coding gene segment sequences; and
(ii) producing the therapeutic or diagnostic antibody that includes the equine variable domain of the antibody expressed by the transgenic rodent.

In one aspect, the equine variable domain is cloned from an antibody expressed by a B cell from the transgenic rodent. In one aspect, the rodent is a mouse. In one aspect, a therapeutic or diagnostic antibody is provided that is produced by a method described herein.

In one aspect, a method is provided for producing a monoclonal antibody that includes an equine variable domain. In one aspect, the method includes:
(i) providing B cells from a transgenic rodent whose genome includes an endogenous rodent immunoglobulin locus variable region which has been deleted and replaced with an heterologous immunoglobulin locus variable region that includes at least one of each of a chimeric V_{H}, D and J_{H} immunoglobulin variable region gene segment at the immunoglobulin heavy chain locus, and/or at least one of each of a chimeric V_{L} and J_{L} variable gene segment at the immunoglobulin light chain loci, wherein each chimeric gene segment includes equine V, D or J immunoglobulin variable region coding sequences embedded in rodent immunoglobulin variable region non-coding gene segment sequences;
(ii) immortalizing the B cells; and
(iii) isolating monoclonal antibodies that include equine variable domains expressed by the immortalized B cells, or genes encoding the antibodies.

In one aspect, the method includes:
(iv) cloning the equine variable domains expressed by the B cells; and
(v) producing a therapeutic or diagnostic antibody that includes the equine variable domain cloned from the B-cells of the transgenic rodent.

In one aspect, a method is provided for producing antibodies that include equine variable domains. In one aspect, the method includes providing a transgenic rodent whose genome includes an endogenous rodent immunoglobulin locus variable region which has been deleted and replaced with an heterologous immunoglobulin locus variable region that includes at least one of each of a chimeric V_{H}, D_{H} and J_{H} immunoglobulin variable region gene segment at the immunoglobulin heavy chain locus, and/or at least one of each of a chimeric V_{L} and J_{L} variable gene segment at the immunoglobulin light chain loci, wherein each chimeric gene segment includes equine V, D or J immunoglobulin variable region coding sequences embedded in rodent immunoglobulin variable region non-coding gene segment sequences, wherein the heterologous immunoglobulin locus of the transgenic rodent expresses antibodies that include equine variable domains.

In one aspect, the method includes isolating the antibodies with equine variable regions expressed by the transgenic rodent, or genes encoding the antibodies. In one aspect, the method includes: (i) obtaining B cells from the transgenic rodent expressing antibodies specific for the target antigen; (ii) immortalizing the B cells; and (iii) isolating antibodies specific for the target antigen from the immortalized B cells.

In one aspect, the method includes cloning equine variable regions from the B cells specific for the particular antigen. In one aspect, the rodent is a mouse. In one aspect, the method includes producing a therapeutic or diagnostic antibody using the equine variable regions cloned from the B cells. In one aspect, a therapeutic or diagnostic antibody is provided that is produced by the method described herein.

These and other aspects, are described in more detail below.

### BRIEF DESCRIPTION OF THE FIGURES

FIG. 1 depicts the mouse Igh locus (top) (including V (IghV), D (IghD), J (IghJ), and C (IghC) gene segments) located at the telomeric end of chromosome 12, the Igk locus (middle) (including V (IgkV), J (IgkJ), and C (IgkC) gene segments) located on located on chromosome 6 and the Igλ locus (bottom) (including IgIV (V), IglJ (J), and IgIC (C) gene segments) located on chromosome 16. Also shown in the Igh locus are 1) PAIR elements, which are cis-regulatory sequences critical for Igh looping to ensure utilization of distal V_{H} gene segments in VDJ rearrangements, 2) the Adam6a male fertility-enabling gene, 3) Intergenic Control Region 1 (IGCR1), which contains sites that regulate ordered, lineage-specific rearrangement of the Igh locus, 4) Eu, the heavy chain intronic enhancer, 5) Su, the switch region, 6) the 3′ regulatory region (3′RR), a cis-acting element that controls isotype switching. Also shown in the Igk locus are the 5′ (E5′) and 3′ (E3′) enhancers, and in the Iga locus are three enhancers, EN 2-4, EN, and Ex 3-1 6).

FIG. 2 is a schematic diagram illustrating the strategy of targeting by homologous recombination to introduce a first set of sequence-specific recombination sites into a region upstream of the H chain variable region gene locus in the genome of a non-equine mammalian host cell.

FIG. 3 is a schematic diagram illustrating the introduction of a second set of sequence-specific recombination sites into a region downstream of the H chain variable region gene locus in the genome of a non-equine mammalian cell via a homology targeting vector. The diagram also illustrates deletion of the endogenous immunoglobulin H chain variable region gene locus as well as the selectable markers from the genome of the non-equine mammalian host cell.

FIG. 4 is a schematic diagram illustrating the RMCE strategy to introduce an heterologous partly equine immunoglobulin H chain locus into the non-equine mammalian host cell genome that has been previously modified to delete the endogenous immunoglobulin H chain variable region locus.

FIG. 5 is a schematic diagram illustrating the introduction of an heterologous partly equine immunoglobulin κ L chain variable region gene locus into the endogenous immunoglobulin κ L chain locus of the mouse genome.

FIGS. 6A and 6B are schematic diagrams illustrating the introduction of an heterologous partly equine immunoglobulin 2 L chain variable region gene locus into the endogenous immunoglobulin A L chain locus of the mouse genome.

### Definitions

The terms used herein are intended to have the plain and ordinary meaning as understood by those of ordinary skill in the art. The following definitions are intended to aid the reader in understanding the present invention but are not intended to vary or otherwise limit the meaning of such terms unless specifically indicated.

The term “locus” as used herein refers to a chromosomal segment or nucleic acid sequence that, respectively, is present endogenously in the genome or is (or about to be) introduced into the genome. For example, an immunoglobulin locus may include part or all of the genes (i.e., V_{H}, D_{H} and J_{H} gene segments or V_{L} and J_{L} gene segments as well as constant region genes) and intervening non-coding sequences (i.e., introns, enhancers, etc.) that support expression of immunoglobulin H or L chain polypeptides. The term “locus” (e.g., immunoglobulin heavy chain variable region locus) may refer to a specific portion of a larger locus (e.g., a portion of the immunoglobulin H chain locus that includes the V_{H}, D_{H} and J_{H} gene segments). Similarly, an immunoglobulin light chain variable region gene locus may refer to a specific portion of a larger locus (e.g., a portion of the immunoglobulin L chain locus that includes the V_{L} and J_{L} gene segments).

The term “immunoglobulin variable region gene” as used herein refers to a variable (V), diversity (D), joining (J) gene segment, including V_{H}, D_{H}, or J_{H} gene segment in the immunoglobulin heavy chain variable region or V_{L} or J_{L} gene segments in the immunoglobulin light chain variable region that encode a portion of an immunoglobulin H or L chain variable domain, respectively. The term “immunoglobulin variable region locus” as used herein refers to part of, or the entire, chromosomal segment or nucleic acid strand containing clusters of V_{H}, D_{H}, or J_{H} gene segments or V_{L} or J_{L} gene segments and the intervening non-coding sequences, including, for example, non-coding regulatory or scaffold sequences.

The term “gene segment” as used herein, refers to a nucleic acid sequence that encodes a part of the heavy chain or light chain variable domain of an immunoglobulin molecule. A gene segment can include coding and non-coding sequences. The coding sequence of a gene segment is a nucleic acid sequence that can be translated into a polypeptide, such the leader peptide and the N-terminal portion of a heavy chain or light chain variable domain. The non-coding sequences of a gene segment are sequences flanking the coding sequence, which may include the promoter, 5′ untranslated sequence, intron intervening the coding sequences of the leader peptide, recombination signal sequence(s) (RSS), and splice sites. The gene segments in the immunoglobulin heavy chain (IGH) locus include the V_{H}, D_{H} and J_{H} gene segments (also referred to as IGHV, IGHD and IGHJ, respectively). The light chain variable region gene segments in the immunoglobulin κ and λ light loci can be referred to as V_{L} and J_{L} gene segments. In the k light chain, the V_{L} and J_{L} gene segments can be referred to as V_{κ} and J_{κ} gene segments or IGKV and IGKJ. Similarly, in the A light chain, the V_{L} and J_{L} gene segments can be referred to as Vi and Ji gene segments or IGLV and IGLJ.

The heavy chain constant region can be referred to as C_{H} or IGHC. The C_{H} region exons in the horse that encode IgM, IgD, IgG1-7, IgE, or IgA can be referred to as, respectively, C_{μ}, C_{δ}, C_{γ1-7}, C_{ε} or C_{α}. Similarly, the immunoglobulin κ or λ constant region can be referred to as C_{κ} or C_{λ}, as well as IGKC or IGLC, respectively.

“Partly equine” as used herein refers to nucleic acids, or their expressed protein and RNA products, that include sequences corresponding to the sequences found in a given locus of both an equine and a non-equine mammalian host. “Partly equine” as used herein also refers to an immunoglobulin locus that includes nucleic acid sequences from both an equine and a non-equine mammal. In one aspect, “partly equine” refers to an immunoglobulin locus that includes, for example, nucleic acid sequences from a rodent, for example, a mouse. In one aspect, the partly equine nucleic acids have coding sequences of equine immunoglobulin H or L chain variable region gene segments and sequences based on the non-coding regulatory or scaffold sequences of the endogenous immunoglobulin locus of the non-equine mammal.

The term “based on” when used with reference to endogenous non-coding regulatory or scaffold sequences from a non-equine mammalian host cell genome refers to the non-coding regulatory or scaffold sequences that are present in the corresponding endogenous locus of the mammalian host cell genome. In one aspect, the term “based on” means that the non-coding regulatory or scaffold sequences that are present in the partly equine immunoglobulin locus share a relatively high degree of homology with the non-coding regulatory or scaffold sequences of the endogenous locus of the host mammal. In one aspect, the non-coding sequences in the partly equine immunoglobulin locus share at least about 80%, about 90%, about 95%, about 96%, about 97%, about 98%, about 99% or about 100% homology with the corresponding non-coding sequences found in the endogenous locus of the host mammal. In one aspect, the non-coding sequences in the partly equine immunoglobulin locus are the same as the corresponding non-coding sequences found in the endogenous locus of the host mammal. In one aspect, the non-coding sequences in the partly equine immunoglobulin locus are retained from an immunoglobulin locus of the host mammal. In one aspect, the non-coding sequences in the partly equine immunoglobulin locus are the same as the corresponding non-coding sequences present in the endogenous locus of the host mammal. In one aspect, the equine coding sequences are embedded in the non-regulatory or scaffold sequences of the immunoglobulin locus of the host mammal. In one aspect, the non-equine host animal is a rodent, such as a rat or mouse.

“Chimeric” refers to a nucleotide sequence that includes nucleotide sequences from two or more species of animal, or a polypeptide, for example, an antibody, encoded by a nucleotide sequence that includes nucleotide sequences from two or more species of animal. A “chimeric” immunoglobulin locus refers to an immunoglobulin locus that includes nucleic acid sequences from two or more species of animal. In one aspect, the chimeric immunoglobulin locus includes equine nucleic acid sequences and mouse nucleic acid sequences. In one aspect, the chimeric immunoglobulin includes protein sequences from two or more species of animal. In one aspect, the chimeric immunoglobulin includes equine sequences and mouse sequences. In one aspect, the chimeric immunoglobulin includes an equine variable domain and a mouse constant domain. In one aspect, the chimeric immunoglobulin variable region locus includes equine V_{H}, D_{H} and J_{H} coding sequences or equine V_{L} and J_{L} coding sequences and non-equine non-coding sequences. In one aspect, the chimeric immunoglobulin variable region locus includes equine V_{H}, D_{H} and J_{H} coding sequences or equine V_{L} and J_{L} coding sequences and mouse non-coding sequences.

“Flanking” as used herein, refers to a sequence, for example, a nucleotide sequence that is upstream or downstream to a reference sequence. In one aspect, the flanking sequence is adjacent to the reference sequence. In one aspect, a pair of sequences flank a reference sequence, such that a first sequence is upstream of the reference sequence and a second sequence is downstream of the reference sequence.

“Endogenous” refers to a nucleic acid sequence or polypeptide that is naturally occurring within an organism or cell.

“Heterologous” refers to a nucleic acid sequence or polypeptide that is not naturally occurring within an organism or cell.

“Non-coding regulatory sequences” refer to sequences that are known to be essential for (i) V(D)J recombination, (ii) isotype switching, (iii) proper expression of the full-length immunoglobulin H or L chains following V(D)J recombination, or (iv) alternate splicing to generate, e.g., membrane and secreted forms of the immunoglobulin H chain. “Non-coding regulatory sequences” may further include the following sequences: enhancer and locus control elements such as the CTCF and PAIR sequences (Proudhon, et al., Adv. Immunol. 128:123-182 (2015)); promoters preceding each endogenous V gene segment; splice sites; introns; or recombination signal sequences flanking each V, D, or J gene segment. In one aspect, the “non-coding regulatory sequences” of the partly equine immunoglobulin locus share at least about 70%, about 75%, about 80%, about 85%, about 90%, about 95%, about 96%, about 97%, about 98%, about 99% and up to about 100% homology with the corresponding non-coding sequences found in the endogenous immunoglobulin locus of the non-equine mammalian host cell. In one aspect, the “non-coding regulatory sequences” of the partly equine immunoglobulin locus have the same sequence as the corresponding non-coding sequences found in the endogenous immunoglobulin locus of the non-equine mammalian host cell.

“Scaffold sequences” refer to sequences intervening the gene segments present in the endogenous immunoglobulin locus of the host cell genome. In certain aspects, the scaffold sequences are interspersed by sequences essential for the expression of a functional non-immunoglobulin gene, for example, ADAM6A or ADAM6B. In one aspect, the scaffold sequences can include a naturally occurring nucleic acid sequence from another species. In one aspect, the scaffolding sequences can be heterologous, based on a naturally occurring nucleic acid sequence from another species. In one aspect, the scaffolding sequences can include an artificial sequence. In one aspect, the scaffold sequence includes sequences that are present in the immunoglobulin locus of the equine genome in combination with other sequences, for example, scaffold sequences from other species. The phrase “non-coding regulatory or scaffold sequence” is inclusive in meaning and can refer to both non-coding regulatory sequences and scaffold sequences in an immunoglobulin locus.

“Specifically binds” refers to the ability of an antibody or immunoglobulin to bind to an epitope or antigenic determinant of a particular antigen with a much higher affinity than the antibody or immunoglobulin binds to other antigens.

The term “homology targeting vector” refers to a nucleic acid sequence used to modify the endogenous genome of a mammalian host cell by homologous recombination. A homology targeting vector can include, for example, targeting sequences with homology to the corresponding endogenous sequences flanking a locus to be modified that is present in the genome of a non-equine mammalian host. In one aspect, the homology targeting vector includes at least one sequence-specific recombination site. In one aspect, the homology targeting vector includes non-coding regulatory or scaffold sequences. In one aspect, the homology targeting vector includes one or more selectable marker genes. In one aspect, the homology targeting vector can be used to introduce a sequence-specific recombination site into particular region of a host cell genome.

“Site-specific recombination” or “sequence-specific recombination” refers to a process of DNA rearrangement between two compatible recombination sequences (also referred to as “sequence-specific recombination sites” or “site-specific recombination sequences”). Site-specific recombination can include any of the following three events: a) deletion of a preselected nucleic acid flanked by the recombination sites; b) inversion of the nucleotide sequence of a preselected nucleic acid flanked by the recombination sites, and c) reciprocal exchange of nucleic acid sequences proximate to recombination sites located on different nucleic acid strands. It is to be understood that this reciprocal exchange of nucleic acid segments can be exploited as a targeting strategy to introduce a heterologous nucleic acid sequence into the genome of a host cell.

The term “targeting sequence” refers to a sequence homologous to DNA sequences in the genome of a cell that flank or are adjacent to the region of an immunoglobulin locus to be modified. The flanking or adjacent sequence may be within the locus itself or upstream or downstream of coding sequences in the genome of the host cell. Targeting sequences are inserted into recombinant DNA vectors which can be used to transfect a host cell, for example, an ES cell, such that sequences to be inserted into the host cell genome, such as the sequence of a recombination site, are flanked by the targeting sequences of the vector.

The term “site-specific targeting vector” as used herein refers to a vector that includes a nucleic acid encoding a sequence-specific recombination site, an heterologous partly equine locus, and optionally a selectable marker gene. In one aspect, the “site-specific targeting vector” is used to modify an endogenous immunoglobulin locus in a host using recombinase-mediated site-specific recombination. The recombination site of the targeting vector is suitable for site-specific recombination with another corresponding recombination site that has been inserted into a genomic sequence of the host cell (e.g., via a homology targeting vector), adjacent to an immunoglobulin locus that is to be modified. Integration of a heterologous partly equine sequence into a recombination site in an immunoglobulin locus results in replacement of the endogenous locus by the heterologous partly equine region.

The term “transgene” is used herein to describe genetic material that has been or is about to be artificially inserted into the genome of a cell, and particularly a cell of a mammalian host animal. The term “transgene” as used herein refers to a partly equine nucleic acid, e.g., a partly equine nucleic acid in the form of a heterologous expression construct or a targeting vector.

“Transgenic animal” refers to a non-equine animal, usually a mammal, having an heterologous nucleic acid sequence present as an extrachromosomal element in a portion of its cells or stably integrated into its germ line DNA (i.e., in the genomic sequence of most or all of its cells). In the present invention, a partly equine nucleic acid is introduced into the germ line of such transgenic animals by genetic manipulation of, for example, embryos or embryonic stem cells of the host animal according to methods well known in the art.

A “vector” includes plasmids and viruses and any DNA or RNA molecule, whether self-replicating or not, that can be used to transform or transfect a cell.

### DETAILED DESCRIPTION OF THE INVENTION

The practice of the techniques described herein may employ, unless otherwise indicated, conventional techniques and descriptions of organic chemistry, polymer technology, molecular biology (including recombinant techniques), cell biology, biochemistry, and sequencing technology, which are within the skill of those who practice in the art. Such conventional techniques include polymer array synthesis, hybridization and ligation of polynucleotides, and detection of hybridization using a label. Specific illustrations of suitable techniques can be had by reference to the examples herein. However, other equivalent conventional procedures can, of course, also be used. Such conventional techniques and descriptions can be found in standard laboratory manuals such as Green, et al., Eds. (1999), Genome Analysis: A Laboratory Manual Series (Vols. I-IV); Weiner, Gabriel, Stephens, Eds. (2007), Genetic Variation: A Laboratory Manual; Dieffenbach and Veksler, Eds. (2007), PCR Primer: A Laboratory Manual; Bowtell and Sambrook (2003), DNA Microarrays: A Molecular Cloning Manual; Mount (2004), Bioinformatics: Sequence and Genome Analysis; Sambrook and Russell (2006), Condensed Protocols from Molecular Cloning: A Laboratory Manual; and Green and Sambrook (2012), Molecular Cloning: A Laboratory Manual (all from Cold Spring Harbor Laboratory Press); Stryer, L. (1995) Biochemistry (4th Ed.) W.H. Freeman, New York N.Y.; Gait, “Oligonucleotide Synthesis: A Practical Approach” 1984, IRL Press, London; Nelson and Cox (2000), Lehninger, Principles of Biochemistry 3.sup.rd Ed., W. H. Freeman Pub., New York, N.Y.; and Berg et al. (2002) Biochemistry, 5.sup.th Ed., W.H. Freeman Pub., New York, N.Y., all of which are herein incorporated in their entirety by reference for all purposes.

Note that as used herein and in the appended claims, the singular forms “a,” “an,” and “the” include plural referents unless the context clearly dictates otherwise. Thus, for example, reference to “a locus” refers to one or more loci, and reference to “the method” includes reference to equivalent steps and methods known to those skilled in the art, and so forth.

As used herein, the term “or” can mean “and/or”, unless explicitly indicated to refer only to alternatives or the alternatives are mutually exclusive. The terms “including,” “includes” and “included”, are not limiting.

Unless defined otherwise, all technical and scientific terms used herein have the same meaning as commonly understood by one of ordinary skill in the art to which this invention belongs. All publications mentioned herein are incorporated by reference for the purpose of describing and disclosing devices, formulations and methodologies that may be used in connection with the presently described invention.

Where a range of values is provided, it is understood that each intervening value, between the upper and lower limit of that range and any other stated or intervening value in that stated range is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

In the following description, numerous specific details are set forth to provide a more thorough understanding of the present invention. However, it will be apparent to one of skill in the art that the present invention may be practiced without one or more of these specific details. In other instances, well-known features and procedures well-known to those skilled in the art have not been described in order to avoid obscuring the invention.

In the humoral immune system, a diverse antibody repertoire is produced by combinatorial and junctional diversity of IgH (Igh) and Igl chain gene loci by a process termed V(D)J recombination. In the developing B cell, the first recombination event to occur is between one D and one J gene segment of the heavy chain locus, and the DNA between these two gene segments is deleted. This D-J recombination is followed by the joining of one V gene segment from a region upstream of the newly formed DJ complex, forming a rearranged VDJ exon. All other sequences between the recombined V and D gene segments of the newly generated VDJ exon are deleted from the genome of the individual B cell. This rearranged exon is ultimately expressed on the B cell surface as the variable region of the H-chain polypeptide, which is associated with an L-chain polypeptide to form the B cell receptor (BCR). The murine and equine Ig loci are highly complex in the numbers of features they contain and in how their coding regions are diversified by V(D)J rearrangement; however, this complexity does not extend to the basic details of the structure of each variable region gene segment. The V, D and J gene segments are uniform in their compositions and organizations. For example, V gene segments have the following features that are arranged in essentially invariant sequential fashion in immunoglobulin loci: a short transcriptional promoter region (<600 bp in length), an exon encoding the majority of the signal peptide for the antibody chain; an intron; an exon encoding a small part of the signal peptide of the antibody chain and the majority of the antibody variable domain, and a 3′ recombination signal sequence necessary for V(D)J rearrangement. Similarly, D gene segments have the following features: a 5′ recombination signal sequence, a coding region and a 3′ recombination signal sequence. The J gene segments have the following features: a 5′ recombination signal sequence, a coding region and a 3′ splice donor sequence.

In one aspect, non-equine mammalian cells are provided that include a heterologous, partly equine nucleic acid sequence that includes equine variable region coding sequences and non-coding regulatory or scaffold sequences present in the immunoglobulin locus of the mammalian host genome, e.g., mouse genomic non-coding sequences when the host mammal is a mouse.

The equine genome V_{H} region includes approximately 50 V_{H}, 40 D_{H} and 8 J_{H} gene segments mapping to a 510 kb region of equine chromosome 24. The lambda (λ) coding region maps to equine chromosome 8, spanning about 1310 kb, and contains approximately 144 Vλ, 7 Jλ and 7 Cλ genes, while the kappa (κ) coding region maps to equine chromosome 15, spanning about 820 kb, and contains approximately 60 Vκ, 4 functional Jκ and 1 Cκ gene. There are several features of the equine Ig loci that are unusual, notably the high frequency of apparently non-functional V gene segments. For example, only 12 of the 50 V_{H} gene segments are functional; 33 are pseudogenes and 5 are classified as open reading frames (ORFs), which are variable gene segments with open reading frames that have defects in splicing sites, recombination signal sequences, regulatory elements, or changes in highly conserved amino acids that are predicted to lead to incorrect folding of the V domain. Similarly, only 27 of the 144 Vλ and 4 of the 7 JA gene segments and 19 of the 60 Vκ gene segments are functional. The genomic structure of the λ locus is also atypical. In humans and mice, for example, there are a cluster (I) of Vλ gene segments followed by a cluster of Jλ-Cλ genes. During B cell development, deletional rearrangement results in the association of one of the Vλ gene segments with one of the JA-CA genes. In the horse, on the other hand, there are a cluster (I) of Vλ gene segments followed by a cluster of Jλ-Cλ genes followed by another cluster (II) of Vλ gene segments. Based on their orientation, the VA gene segments in cluster II undergo inversional V→J gene rearrangement, which occurs much less frequently than deletional gene rearrangement, to create a Vλ exon that includes the recombined Vλ and JA gene segments. Moreover, of the 34 Vλ gene segments in cluster II, 25 are pseudogenes and 2 are ORFs, although Walther et al. (Dev. Comp. Immunol. 3:303 (2015)) have identified seven functional Vλ gene segments in this cluster. Analysis of the sequence of this contig (NW_001867428.1), indicates that none of the seven putative functional Vλ gene segments in cluster II include a conventional RSS, which makes it unlikely that they are used in the equine λLC repertoire. However, as shown in Table 1, all seven of these Vλ gene segments are found as cDNAs in GenBank, indicating that they can be rearranged and expressed in B cells. In one aspect, the partly equine Vλ locus described herein can include Vλ gene segments from both cluster I and cluster II. In one aspect, in the partly equine H and K and A L chain loci, all V_{H}, D_{H} and J_{H} segments and all V_{L} and J_{L} segments are flanked by mouse RSS to promote rearrangement during B cell development and contribution to the partly equine antibody repertoire of the transgenic mouse.

As with humans and mice, horses express two types of Ig light chains (K and A). However, the K to A ratio differs significantly among these animals. In mice, approximately 96% of light chains in the serum antibodies are the k type, while the k type in humans accounts for only 66% of the total population of Ig L chains. In contrast, the L chain repertoire in horses is dominated (95%) by 2.

The partly equine nucleic acid sequences incorporated into the Igh, Igk or Igλ loci allow the transgenic animal to produce antibodies that include equine heavy chain variable regions paired with equine K or λ variable regions. The partly equine immunoglobulin variable region locus retains the regulatory sequences and other elements within the intervening sequences of the host genome (e.g., rodent) that help to promote efficient antibody production and antigen recognition in the host.

In one aspect, a synthetic, or recombinantly produced, partly equine immunoglobulin locus is provided that includes equine coding sequences and non-equine non-coding regulatory or scaffold sequences from an immunoglobulin V_{H}, Vλ or Vκ locus.

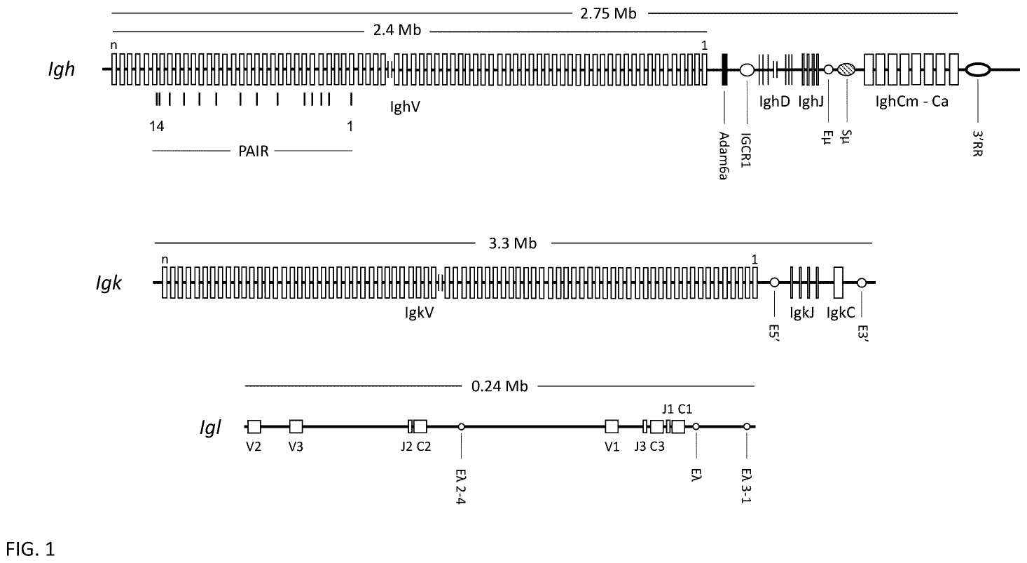

In one aspect the synthetic H chain DNA segment contains one or more of the following elements: the ADAM6 gene needed for male fertility, Pax-5-Activated Intergenic Repeats (PAIR) elements involved in Igh locus contraction, CTCF binding sites from the heavy chain intergenic control region 1, involved in regulating normal VDJ rearrangement ((Proudhon, et al., Adv. Immunol., 128:123-182 (2015)), or combinations thereof. The locations of these endogenous non-coding regulatory and scaffold sequences in the mouse Igh locus are depicted in FIG. 1, which illustrates from left to right: the ˜100 functional heavy chain variable region gene segments (101); PAIR, Pax-5 Activated Intergenic Repeats involved in Igh locus contraction for VDJ recombination (102); Adam6a, a disintegrin and metallopeptidase domain 6A gene required for male fertility (103); Pre-D region, a 21609 bp fragment upstream of the most distal D_{H} gene segment, Ighd-5 (104); Intergenic Control Region 1 (IGCR1) that contains CTCF insulator sites to regulate V_{H} gene segment usage (106); D_{H}, diversity gene segments (10-15 depending on the mouse strain) (105); four joining J_{H} gene segments (107); Eu, the intronic enhancer involved in VDJ recombination (108); Su, the u switch region for isotype switching (109); eight heavy chain constant region genes: Cμ, Cδ, Cγ3, Cγ1, Cγ2b, C2γa/c, Ce, and Cα (110); 3′ Regulatory Region (3′RR) that controls isotype switching and somatic hypermutation (111). FIG. 1 is modified from a figure taken from Proudhon, et al., Adv. Immunol., 128:123-182 (2015).

In one aspect, the heterologous partly equine immunoglobulin locus to be integrated into a mammalian host cell includes all or a substantial number of the known equine V_{H} gene segments. In some instances, however, it may be desirable to use a subset of such V_{H} gene segments. In one aspect, even as few as one equine V_{H} coding sequence may be included in the partly equine immunoglobulin locus.

In one aspect, the non-equine mammals or mammalian cell includes a heterologous partly equine immunoglobulin locus that includes equine V_{H}, D_{H}, and J_{H} gene coding sequences. In one aspect, the partly equine immunoglobulin locus includes non-coding regulatory and scaffold sequences, for example, pre-D sequences, based on the endogenous Igh locus of the non-equine mammalian host. In one aspect, the heterologous partly equine immunoglobulin locus includes a fully recombined V(D)J exon.

In one aspect, the transgenic non-equine mammal is a rodent, for example, a mouse, that includes a heterologous, partly equine immunoglobulin locus that includes equine V_{H}, D_{H}, and J_{H} genes and intervening sequences, including, for example, a pre-D region, based on the intervening (non-coding regulatory or scaffold) sequences in the rodent. In one aspect, the transgenic rodent further includes a partly equine Igl loci that include equine Vκ or Vλ coding sequences, and equine Jκ or Jλ coding sequences, respectively, and intervening sequences, such as non-coding regulatory or scaffold sequences present in the Igl loci of the rodent.

In one aspect, the entire endogenous V_{H} immunoglobulin locus of the mouse genome is deleted and replaced with 12 functional equine V_{H} gene segments and non-coding sequences of the J558 V_{H} locus of the mouse genome. In one aspect, the heterologous immunoglobulin locus includes 40 equine D_{H} and 8 J_{H} gene segments. In one aspect, the heterologous immunoglobulin locus includes the mouse pre-D region. In one aspect, the equine V_{H}, D_{H}, and J_{H} coding sequences are embedded in the rodent non-coding sequences.

In one aspect, a combination of homologous recombination and site-specific recombination is used to generate transgenic cells and animals. In one aspect, a homology targeting vector is used to introduce sequence-specific recombination sites into a mammalian host cell genome at a desired location in the endogenous immunoglobulin loci. In one aspect, the sequence-specific recombination site is inserted into the genome of a mammalian host cell by homologous recombination and does not affect expression or coding sequences of any other genes in the mammalian host cell. In one aspect, the ability of the immunoglobulin genes to be transcribed and translated to produce antibodies is maintained after the recombination sites and, optionally, any additional sequence such as a selectable marker gene are inserted. However, in some cases it is possible to insert other heterologous sequences into an immunoglobulin locus sequence such that an amino acid sequence of the resultant antibody molecule is altered by the insertion, but the antibody retains sufficient functionality for the desired purpose. In one aspect, one or more polymorphisms are introduced into the endogenous locus in the constant region exons, thereby providing an allotypic marker so that the different Ig alleles can be distinguished.

In one aspect, the homology targeting vector is used to replace sequences within the endogenous immunoglobulin locus as well as to insert sequence-specific recombination sites and one or more selectable marker genes into the host cell genome. It is understood by those of ordinary skill in the art that a selectable marker gene as used herein can be exploited identify and eliminate cells that have not undergone homologous recombination or cells that harbor random integration of the targeting vector.

Methods for homologous recombination are known and include those described in U.S. Pat. Nos. 6,689,610; 6,204,061; 5,631,153; 5,627,059; 5,487,992; and 5,464,764, each of which is incorporated by reference in its entirety.

### Site/Sequence-Specific Recombination

Site/sequence-specific recombination differs from homologous recombination in that short, specific DNA sequences, which are required for recognition by a recombinase, are the only sites at which recombination occurs. Depending on the orientations of these sites on a particular DNA strand or chromosome, the specialized recombinases that recognize these specific sequences can catalyze i) DNA excision or ii) DNA inversion or rotation. Site-specific recombination can also occur between two DNA strands if these sites are not present on the same chromosome. A number of bacteriophage- and yeast-derived site-specific recombination systems, each including a recombinase and its cognate recognition sites, have been shown to work in eukaryotic cells, including, but not limited to, the bacteriophage P1 Cre/lox system, the yeast FLP-FRT system, and the Dre system of the tyrosine family of site-specific recombinases. Such systems and methods are described, e.g. in U.S. Pat. Nos. 7,422,889; 7,112,715; 6,956,146; 6,774,279; 5,677,177; 5,885,836; 5,654,182; and 4,959,317; each of which is incorporated herein by reference.

Other systems of the tyrosine family of site-specific recombinases can be used, including, but not limited to, bacteriophage lambda integrase, HK2022 integrase, and systems belonging to the serine family of recombinases, including, for example, bacteriophage phiC31, and R4Tp901 integrases.

Because site-specific recombination can occur between two different DNA strands, site-specific recombination can be used to introduce a heterologous immunoglobulin locus into a host cell genome by a process called recombinase-mediated cassette exchange (RMCE). The RMCE process can be exploited using wild-type and mutant sequence-specific recombination sites for a recombinase protein. In one aspect, RMCE includes negative selection. For example, a chromosomal locus to be targeted may be flanked by a wild-type LoxP site on one end and by a mutant LoxP site on the other. Likewise, a vector can include a heterologous sequence to be inserted into the host cell genome that is flanked by a wild-type LoxP site on one end and by a mutant LoxP site on the other. When the vector is transfected into the host cell in the presence of Cre recombinase, Cre recombinase will catalyze RMCE between the endogenous DNA strands and the DNA of the vector, rather than catalyzing an excision reaction on the same DNA strands, because the wild-type LoxP and mutant LoxP sites on each DNA strand are incompatible for recombination with each other. As such, the LoxP site on one DNA strand will only recombine with a LoxP site on the other DNA strand; and similarly, the mutated LoxP site on one DNA strand will only recombine with a mutated LoxP site on the other DNA strand.

In one aspect, variants of the sequence-specific recombination sites that are recognized by the same recombinase for RMCE are used. Examples of such sequence-specific recombination site variants include those that contain a combination of inverted repeats or those that include recombination sites with mutant spacer sequences. For example, two classes of variant recombinase sites are available to engineer stable Cre-loxP integrative recombination. Both exploit sequence mutations in the Cre recognition sequence, either within the 8 bp spacer region or the 13-bp inverted repeats. Spacer mutants such as lox511 (Hoess, et al., Nucleic Acids Res, 14:2287-2300 (1986)), lox5171 and lox2272 (Lee and Saito, Gene, 216:55-65 (1998)), m2, m3, m7, and m11 (Langer, et al., Nucleic Acids Res, 30:3067-3077 (2002)) recombine readily with themselves but have a markedly reduced rate of recombination with the wild-type site. This class of mutants has been exploited for DNA insertion by RMCE using non-interacting Cre-Lox recombination sites and non-interacting FLP recombination sites (Baer and Bode, Curr Opin Biotechnol, 12:473-480 (2001); Albert, et al., Plant J, 7:649-659 (1995); Seibler and Bode, Biochemistry, 36:1740-1747 (1997); Schlake and Bode, Biochemistry, 33:12746-12751 (1994)).

Inverted repeat mutants are another class of variant recombinase sites. For example, LoxP sites can contain altered bases in the left inverted repeat (LE mutant) or the right inverted repeat (RE mutant). An LE mutant, lox71, has 5 bp on the 5′ end of the left inverted repeat that is changed from the wild type sequence to TACCG (Araki, et al, Nucleic Acids Res, 25:868-872 (1997)). Similarly, the RE mutant, lox66, has the five 3′-most bases changed to CGGTA. Inverted repeat mutants are used for integrating plasmid inserts into chromosomal DNA with the LE mutant designated as the “target” chromosomal loxP site into which the “donor” RE mutant recombines. Post-recombination, loxP sites are located in cis, flanking the inserted segment. The mechanism of recombination is such that, post-recombination, one loxP site is a double mutant (containing both the LE and RE inverted repeat mutations) and the other is wild type (Lee and Sadowski, Prog Nucleic Acid Res Mol Biol, 80: 1-42 (2005); Lee and Sadowski, J Mol Biol, 326:397-412 (2003)). The double mutant is sufficiently different from the wild-type site that it is unrecognized by Cre recombinase and the inserted segment is not excised.

In one aspect, sequence-specific recombination sites are introduced into introns, rather than coding or regulatory sequences to avoid disrupting regulatory sequences or coding sequences used in antibody expression.

Introduction of the sequence-specific recombination sites may be achieved by conventional homologous recombination techniques. Such techniques are described in references such as e.g., Green and Sambrook (2012) (Molecular cloning: a laboratory manual 4th ed. (Cold Spring Harbor, N.Y.: Cold Spring Harbor Laboratory Press) and Nagy, A. (2003). (Manipulating the mouse embryo: a laboratory manual, 3rd ed. (Cold Spring Harbor, N.Y.: Cold Spring Harbor Laboratory Press).

Specific recombination into the genome can be facilitated using vectors designed for positive or negative selection as known in the art. In order to facilitate identification of cells that have undergone the replacement reaction, an appropriate genetic marker system may be employed and cells selected by, for example, use of a selection tissue culture medium. In one aspect, nucleic acid sequences at or adjacent to the two end points of the heterologous sequence, for example, a marker system or gene can be removed following selection of the cells containing the heterologous nucleic acid.

In one aspect, cells in which the endogenous immunoglobulin locus has been deleted may be positively selected for using a marker gene, which can optionally be removed from the cells following or as a result of the recombination event. A positive selection system that may be used is based on the use of two non-functional portions of a marker gene, such as Hypoxanthine-guanine phosphoribosyltransferase (HPRT), that are brought together through the recombination event. In one aspect, the two non-functional portions are brought into functional association upon a successful replacement of the endogenous immunoglobulin locus with the heterologous immunoglobulin locus. In one aspect, the functionally reconstituted marker gene is flanked on either side by further sequence-specific recombination sites (which are different from the sequence-specific recombination sites used for the replacement reaction), such that the marker gene can be excised from the genome, using an appropriate site-specific recombinase. In another aspect, cells are negatively selected against upon exposure to a toxin or drug. For example, cells in which a targeting construct is not integrated by homologous recombination but is randomly integrated into the genome will retain expression of Herpes Simplex Virus-Thymidine Kinase (HSV-TK) if the HSV-TK gene is located outside of the region of homology. Such cells can be selected against using nucleoside analogues such as ganciclovir.

In one aspect, the recombinase is provided as a purified protein. In one aspect, the recombination is provided as a protein expressed from a vector construct transiently transfected into the host cell or stably integrated into the host cell genome. Alternatively, a transgenic animal that includes the heterologous immunoglobulin locus may be crossed with an animal that expresses the recombinase.

In one aspect, two or more sets of sequence-specific recombination sites are included within the engineered genome, such that multiple rounds of RMCE can be exploited to insert the partly equine immunoglobulin variable region locus into a non-equine mammalian host cell genome.

In one aspect, the partly equine immunoglobulin locus is introduced using CRISPR technology. For example, the CRISPR/Cas9 genome editing system may be used for targeted recombination (He, et al., Nuc. Acids Res., 44:e85, (2016)).

### Generation of Transgenic Animals

In one aspect, methods are provided for the creation of transgenic animals, for example, rodents, for example, mice, that include a heterologous partly equine immunoglobulin locus.

In one aspect, the genome of the transgenic animal is modified so that B cells of the transgenic animal are capable of expressing more than one functional V_{H} domain per cell, i.e., the cells produce bispecific antibodies as described in WO20170/35252, filed Aug. 24, 2016, entitled “Enhanced Production of Immunoglobulins”, the disclosure of which is incorporated by reference herein.

In one aspect, the genome of the transgenic animal is modified so that B cells of the transgenic animal are capable of expressing antibodies that include heavy chains but no light chains, i.e., the cells produce heavy chain-only antibodies.

In one aspect, the host cell is an embryonic stem (ES) cell, which can then be used to create a transgenic mammal. In one aspect, the method includes: isolating an embryonic stem cell that includes the heterologous partly equine immunoglobulin locus and using the ES cell to generate a transgenic animal that contains the heterologous partly equine immunoglobulin locus.

### EXAMPLES

The following examples are put forth to provide those of ordinary skill in the art with a complete disclosure and description of how to make and use the present invention, and are not intended to limit the scope of what the inventors regard as their invention, nor are they intended to represent or imply that the experiments below are all of or the only experiments performed. It will be appreciated by persons skilled in the art that numerous variations or modifications may be made without departing from the spirit or scope of the invention as described herein. The examples are, therefore, to be considered as illustrative and not restrictive.

Efforts have been made to ensure accuracy with respect to terms and numbers used (e.g., vectors, amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric.

The examples illustrate targeting by both a 5′ vector and a 3′ vector that flank a site of recombination and introduction of synthetic DNA via RMCE. Upon reading the specification, it will be apparent to one skilled in art that the 5′ vector targeting can take place first followed by the 3′, or the 3′ vector targeting can take place first followed by the 5′ vector. In some circumstances, targeting can be carried out simultaneously with dual detection mechanisms. Although some different strategies are used in each example to select for cells that have properly integrated the 5′ or 3′ vector, it will also be apparent that, with minor modifications, such strategies are interchangeable for targeting the Igh, Igκ or Igλloci.

### Example 1: Introduction of an Heterologous Partly Equine Immunoglobulin Variable Region Gene Locus into the Immunoglobulin H Chain Variable Region Gene Locus of a Non-Equine Mammalian Host Cell Genome

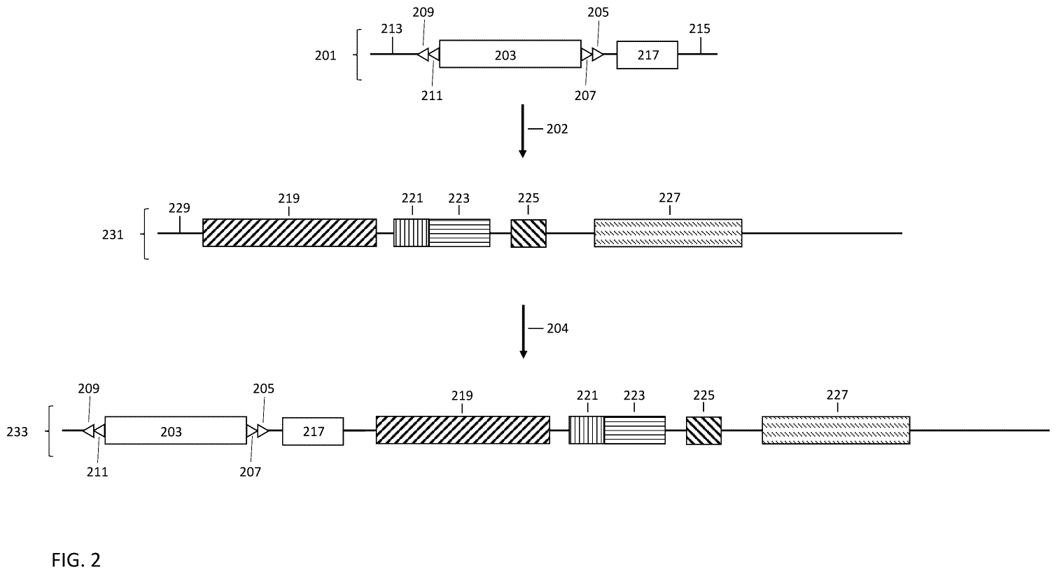

An exemplary method illustrating the introduction of a heterologous partly equine immunoglobulin locus into the genomic locus of a non-mammalian ES cell is illustrated in FIGS. 2-4. FIG. 2 depicts a method for introducing site-specific recombination sequences upstream (5′) of the endogenous V_{H} gene segments. A 5′ homology targeting vector (201) is provided that includes a puromycin phosphotransferase-thymidine kinase fusion protein (puro-TK) (203) flanked by two different recombinase recognition sites (e.g., FRT (207) and loxP (205) for Flp and Cre, respectively) and two different mutant sites (e.g., modified mutant FRT (209) and mutant loxP (211)) that lack the ability to recombine with their respective wild-type counterparts/sites (i.e., wild-type FRT (207) and wild-type loxP (205)). The targeting vector includes a diphtheria toxin receptor (DTR) cDNA (217) for use in negative selection of cells. The targeting vector also optionally includes a visual marker such as a green fluorescent protein (GFP) (not shown). The regions 213 and 215 are homologous to the 5′ and 3′ portions, respectively, of a contiguous region (229) in the endogenous non-equine locus that is 5′ of the genomic region that includes the endogenous non-equine V_{H} gene segments (219). The homology targeting vector (201) is introduced (202) into the ES cell, which has an immunoglobulin locus (231) that includes endogenous V_{H} gene segments (219), the pre-D region (221), the D_{H} gene segments (223), J_{H} gene segments (225), and the immunoglobulin constant gene region genes (227). The site-specific recombination sequences and the DTR cDNA from the homology targeting vector (201) are integrated (204) into the non-equine genome at a site 5′ of the endogenous mouse V_{H} gene locus, resulting in the genomic structure illustrated at 233.

Mouse embryonic stem (ES) cells (derived from C57B1/6NTac mice) are transfected by electroporation with the 5′ vector (201) according to known procedures. Prior to electroporation, the vector DNA is linearized with a rare-cutting restriction enzyme that cuts only in the prokaryotic plasmid sequence or the polylinker associated with it. The transfected cells are plated and after ˜24 hours they are placed under selection for cells that have integrated the 5′ vector into their DNA. The ES cells that do not have the 5′ vector (201) integrated into their genome can be selected against (killed) by including puromycin in the culture medium; only the ES cells that have stably integrated the 5′ vector (201) into their genome and constitutively express the puro-TK gene are resistant to puromycin.

Colonies of drug-resistant ES cells are physically extracted from their plates after they become visible to the naked eye about a week later. These picked colonies are disaggregated, re-plated in micro-well plates, and cultured for several days. Thereafter, each of the clones of cells is divided such that some of the cells can be frozen as an archive, and the rest used for isolation of DNA for analytical purposes. The primary screening procedure for the introduction of 5′ vector can be carried out by Southern blotting, or by PCR with confirmations from secondary screening methods such as Southern blotting.

DNA from the ES cell clones is screened by PCR using a widely practiced gene-targeting assay design. For this assay, one of the PCR oligonucleotide primer sequences maps outside the region of identity shared between the 5′ vector (201) and the genomic DNA, while the other maps within the 5′ vector, e.g., in the Puro-TK gene (203). According to the standard design, these assays detect DNA that would only be present in clones of ES cells that undergo homologous recombination between the 5′ targeting vector and the endogenous mouse Igh locus.

The Southern blot assays are performed according to widely used procedures using three probes and genomic DNA digested with multiple restriction enzymes chosen so that the combination of probes and digests allow the structure of the targeted locus in the clones to be identified as properly modified by homologous recombination. One of the probes maps to DNA sequence flanking the 5′ side of the region of identity shared between the 5′ targeting vector and the genomic DNA; a second probe maps outside the region of identity but on the 3′ side; and the third probe maps within the novel DNA between the two arms of genomic identity in the vector, e.g., in the Puro-TK gene (203). The Southern blot identifies the presence of the expected restriction enzyme-generated fragment of DNA corresponding to the modified sequence, i.e., by homologous recombination with the 5′ targeting vector, part of the Igh locus as detected by one of the external probes and by the Puro-TK probe. The external probe detects the mutant fragment and also a wild-type fragment from the non-mutant copy of the immunoglobulin Igh locus on the homologous chromosome.

Karyotypes of PCR- and Southern blot-positive clones of ES cells are analyzed using an in situ fluorescence hybridization procedure designed to distinguish the most commonly arising chromosomal aberrations that arise in mouse ES cells. Clones with such aberrations are excluded from further use. ES cell clones that have the expected genomic structure based on the Southern blot data, and that do not have detectable chromosomal aberrations based on the karyotype analysis, are selected for further use.

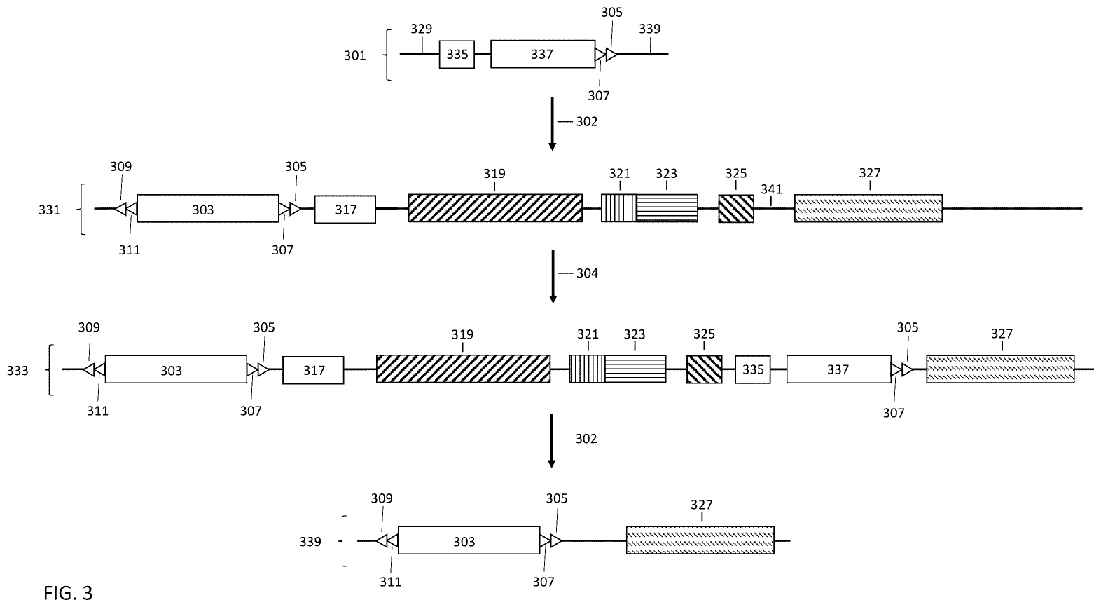

As illustrated in FIG. 3, a 3′ homology targeting vector (301) is provided that includes an optional hypoxanthine-guanine phosphoribosyltransferase (HPRT) gene (335) that can be used for positive selection in HPRT-deficient ES cells; a neomycin resistance gene (337); recombinase recognition sites FRT (307) and loxP (305), for Flp and Cre, respectively. The regions 329 and 339 are homologous to the 5′ and 3′ portions, respectively, of a contiguous region (341) in the endogenous mouse locus that is downstream of the endogenous J_{H} gene segments (325) and upstream of the constant region genes (327). The homology targeting vector is introduced (302) into the modified mouse immunoglobulin locus (331), which includes the endogenous V_{H} gene segments (319), the pre-D region (321), the D gene segments (323), the J_{H} gene segments (325), and the constant region genes (327). The site-specific recombination sequences (307, 305), the HPRT gene (335) and a neomycin resistance gene (337) of the homology targeting vector are integrated (304) into the mouse genome upstream of the endogenous mouse constant region genes (327), resulting in the genomic structure illustrated at 333.

Acceptable clones modified with the 3′ vector (301) are identified using procedures and screening assays that are essentially identical in design to those used with the 5′ vector (201) except that neomycin or HPRT selection is used instead of puromycin for selection. The PCR assays, probes and digests are also tailored to match the genomic region modified by the 3′ vector. Karyotypes of PCR- and Southern blot-positive clones of ES cells are analyzed using an in situ fluorescence hybridization procedure designed to distinguish the most commonly arising chromosomal aberrations that arise in mouse ES cells. Clones with such aberrations are excluded from further use.

Clones of ES cells that have been mutated by both the 3′ and the 5′ vectors, i.e., doubly targeted cells carrying both engineered mutations, are isolated following vector targeting and analysis. The clones must have undergone gene targeting on the same chromosome, as opposed to homologous chromosomes (i.e., the engineered mutations created by the targeting vectors must be in cis on the same DNA strand rather than in trans on separate homologous DNA strands). Clones with the cis arrangement are distinguished from those with the trans arrangement by analytical procedures such as fluorescence in situ hybridization of metaphase spreads using probes that hybridize to the novel DNA present in the two gene targeting vectors (303 and 337) between their arms of genomic identity. The two types of clones can also be distinguished from one another by transfecting them with a vector expressing Cre recombinase, which deletes the HPRT (335) and neomycin resistance (337) genes if the targeting vectors have been integrated in cis, and then analyzing the drug resistance phenotype of the clones by a “sibling selection” screening procedure in which some of the cells from each clone are tested for resistance to G418/neomycin. The majority of the resulting cis-derived clones are also sensitive to G418/neomycin, in contrast to the trans-derived clones, which should retain resistance to the drugs. Doubly targeted clones of cells with the cis-arrangement of engineered mutations in the heavy chain locus are selected for further use.

Once the two recombination sites are integrated into the mammalian host cell genome, the endogenous immunoglobulin locus is then subjected to recombination by introducing one of the recombinases corresponding to the sequence-specific recombination sites integrated into the genome, e.g., either Flp or Cre. In the presence of Flp or Cre (302), all the intervening sequences between the wild-type FRT or wild-type LoxP sites including the DTR gene (317), the endogenous Igh variable region gene loci (319, 323, 325), the pre-D region (321), and the HPRT (335) and neomycin resistance (337) genes are deleted, resulting in a genomic structure illustrated at 339. The procedure depends on the second targeting having occurred on the same chromosome rather than on its homolog (i.e., in cis rather than in trans). If the targeting occurs in cis as intended, the cells are not sensitive to negative selection by diphtheria toxin introduced into the media, because the DTR gene (317) that causes sensitivity to diphtheria toxin should be absent (deleted) from the host cell genome. Likewise, ES cells that harbor random integration of the first or second targeting vector(s) are rendered sensitive to diphtheria toxin by presence of the undeleted DTR gene.

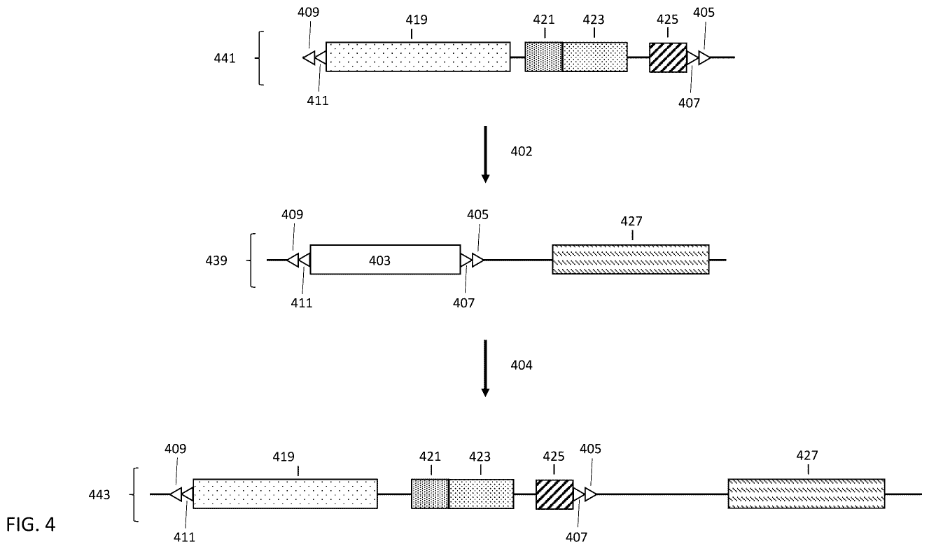

ES cell clones carrying the sequence deletion in one of the two homologous copies of their immunoglobulin heavy chain locus are retransfected with a Cre recombinase expression vector and a vector that includes a partly equine immunoglobulin heavy chain locus containing equine V_{H}, D_{H} and J_{H} gene segment coding sequences embedded in mouse non-coding sequences. FIG. 4 illustrates introduction of the heterologous partly equine immunoglobulin heavy chain locus into a mouse genome in which the part of the endogenous immunoglobulin heavy chain locus that encodes the heavy chain variable region domains has been deleted, including the intervening sequences between the endogenous V_{H} and J_{H} gene loci. A site-specific targeting vector (441) that includes a partly equine immunoglobulin locus to be inserted into the non-equine host genome is introduced (402) into the modified genome of the host cell (439) by RMCE. The site-specific targeting vector (441) that includes a partly equine V_{H} gene locus (419), mouse pre-D region (421), a partly equine D_{H} gene locus (423), a partly equine J_{H} gene locus (425), as well as flanking mutant FRT (409), mutant LoxP (411) wild-type FRT (407) and wild-type LoxP (405) sites is introduced (402) into the host cell by RMCE. Specifically, the partly equine V_{H} gene locus (419) includes 12 functional equine V_{H} gene segment coding sequences and 3′ non-equine RSS and intervening sequences present in the endogenous non-equine genome; the pre-D region (421) includes a 21.6 kb non-equine sequence present upstream in the endogenous non-equine genome; the D_{H} region (423) includes codons of 40 equine D_{H} gene segments flanked by non-equine RSS and embedded in the intervening sequences present in the endogenous non-equine D_{H} region; and the J_{H} gene locus (425) includes codons of 8 equine J_{H} gene segments with 5′ non-equine RSS and embedded in the intervening sequences present in the endogenous non-equine genome. In one aspect, the Igh locus of the host cell genome is modified to delete all endogenous V_{H}, D_{H}, and J_{H} gene segments including the intervening sequences as described in relation to FIG. 3. As a consequence of this modification, the endogenous non-equine Igh locus (439) is left with a puro-TK fusion gene (403), which is flanked by a mutant FRT site (409) and a mutant LoxP site (411) upstream as well as a wild-type FRT (407) and a wild-type LoxP (405) downstream. Upon introduction of the appropriate recombinase (404), the partly equine immunoglobulin locus is integrated between the lox5171 (411) and loxP (405) sites into the genome upstream of the endogenous mouse constant region genes (427), to create the DNA region illustrated at 443.

ES cells that have not undergone RMCE and integration of the partly equine Igh locus retain the puro-TK fusion gene (403) and are eliminated by inclusion of ganciclovir to the tissue culture media.

The sequences of equine V_{H}, D_{H} and J_{H} gene segments are shown in SEQ ID NO. 1-65.

Integration of the heterologous partly equine immunoglobulin region can be detected by Southern blotting, or by PCR with confirmations from secondary screening methods such as Southern blotting. The screening methods are designed to detect the presence of the inserted V_{H}, D_{H} or J_{H} gene loci, as well as the intervening sequences. Karyotypes of PCR- and Southern blot-positive clones of ES cells are analyzed using an in situ fluorescence hybridization procedure designed to distinguish the most commonly arising chromosomal aberrations that arise in mouse ES cells. Clones with such aberrations are excluded from further use

ES cell clones carrying the partly equine immunoglobulin heavy chain variable region (443) in the mouse heavy chain locus are microinjected into mouse blastocysts from strain DBA/2 to create ES cell-derived chimeric mice according to standard procedures. Male chimeric mice with the highest levels of ES cell-derived contribution to their coats are selected for mating to female mice. Offspring from these matings are analyzed for the presence of the partly equine immunoglobulin heavy chain locus. Mice that carry the partly equine immunoglobulin heavy chain locus are used to establish a colony of mice.

### Example 2: Introduction of an Heterologous Partly Equine Immunoglobulin Locus into the Immunoglobulin Kappa Chain Gene Locus of a Mouse Genome

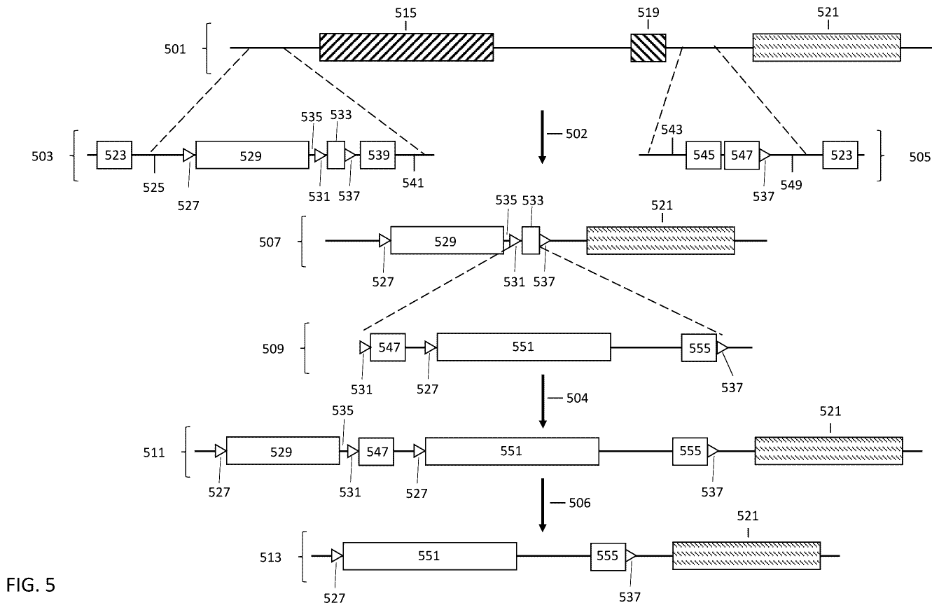

A method for replacing a portion of a mouse Igκ locus with partly equine Igκ locus is illustrated in FIG. 5. This method includes introducing a first site-specific recombinase recognition sequence into the mouse genome, which may be introduced either 5′ or 3′ of the cluster of endogenous V_{K} (515) and J_{K} (519) region gene segments of the mouse genome, followed by the introduction of a second site-specific recombinase recognition sequence into the mouse genome, which in combination with the first sequence-specific recombination site, flanks the entire locus that includes clusters of V_{K} and J_{K} gene segments upstream of the constant region gene (521). The flanked region is deleted and replaced with a partly equine immunoglobulin light chain variable region locus using the relevant site-specific recombinase.

The targeting vectors employed for introducing the site-specific recombination sequences on either side of the V_{K} (515) and J_{K} (519) gene segments also include an additional site-specific recombination sequence that is modified so that it is still recognized efficiently by the recombinase but does not recombine with unmodified sites. This site is positioned in the targeting vector such that after deletion of the V_{K} and J_{K} gene segment clusters it can be used for a second site specific recombination event in which a heterologous immunoglobulin light chain variable region locus is inserted into the modified V_{K} locus via RMCE. In this example, the heterologous immunoglobulin light chain variable region locus is a synthetic nucleic acid that includes equine V_{K} and J_{K} gene segments and mouse Igκ variable region non-coding sequences.

Two gene targeting vectors are constructed to accomplish the process just outlined. One of the vectors (503) includes mouse genomic DNA (525 and 541) taken from the 5′ end of the locus, upstream of the most distal V_{K} gene segment. The other vector (505) includes mouse genomic DNA (543 and 549) taken from within the locus downstream (3′) of the J_{K} gene segments (519) and upstream of the constant region gene (521).

The key features of the 5′ vector (503) are as follows: a gene encoding the diphtheria toxin A subunit (DTA) under transcriptional control of a modified herpes simplex virus type I thymidine kinase gene promoter coupled to two mutant transcriptional enhancers from the polyoma virus (523); 6 Kb of mouse genomic DNA (525) mapping upstream of the most distal variable region gene in the kappa chain locus; a FRT recognition sequence for the Flp recombinase (527); a piece of genomic DNA containing the mouse Polr2a gene promoter (529); a translation initiation sequence (535, methionine codon embedded in a “Kozak” consensus sequence); a mutated loxP recognition sequence (lox5171) for the Cre recombinase (531); a transcription termination/polyadenylation sequence (533); a loxP recognition sequence for the Cre recombinase (537); a gene encoding a fusion protein included of a protein conferring resistance to puromycin fused to a truncated form of the thymidine kinase (pu-TK) under transcriptional control of the promoter from the mouse phosphoglycerate kinase 1 gene (539); 2.5 Kb of mouse genomic DNA (541) mapping close to the 6 Kb sequence at the 5′ end in the vector and arranged in the native relative orientation.

The key features of the 3′ vector (505) are as follows: 6 Kb of mouse genomic DNA (543) mapping within the intron between the J_{κ} (519) and C_{κ} (521) gene loci; a gene encoding the human hypoxanthine-guanine phosphoribosyl transferase (HPRT) under transcriptional control of the mouse Polr2a gene promoter (545); a neomycin resistance gene under the control of the mouse phosphoglycerate kinase 1 gene promoter (547); a loxP recognition sequence for the Cre recombinase (537); 3.6 Kb of mouse genomic DNA (549) that maps immediately downstream in the genome of the 6 Kb DNA fragment included at the 5′ end in the vector, with the two fragments oriented in the same relative way as in the mouse genome; a gene encoding the diphtheria toxin A subunit (DTA) under transcriptional control of a modified herpes simplex virus type I thymidine kinase gene promoter coupled to two mutant transcriptional enhancers from the polyoma virus (523).

Mouse embryonic stem (ES) cells derived from C57B1/6NTac mice are transfected by electroporation with the 3′ vector (505) according to known procedures. Prior to electroporation, the vector DNA is linearized with a rare-cutting restriction enzyme that cuts only in the prokaryotic plasmid sequence or the polylinker associated with it. The transfected cells are plated and after ˜24 hours they are placed under positive selection for cells that have integrated the 3′ vector into their DNA by using the neomycin analogue drug G418. There is also negative selection for cells that have integrated the vector into their DNA but not by homologous recombination. Non-homologous recombination will result in retention of the DTA gene, which will kill the cells when the gene is expressed, whereas the DTA gene is deleted by homologous recombination since it lies outside of the region of vector homology with the mouse Igκ locus. Colonies of drug-resistant ES cells are physically extracted from their plates after they become visible to the naked eye about a week later. These picked colonies are disaggregated, re-plated in micro-well plates, and cultured for several days. Thereafter, each of the clones of cells is divided such that some of the cells could be frozen as an archive, and the rest used for isolation of DNA for analytical purposes.

DNA from the ES cell clones is screened by PCR using a gene-targeting assay. For this assay, one of the PCR oligonucleotide primer sequences maps outside the region of identity shared between the 3′ vector (505) and the genomic DNA (501), while the other maps within the novel DNA between the two arms of genomic identity in the vector, e.g., in the HPRT (545) or neomycin resistance (547) genes. These assays detect pieces of DNA that are only present in clones of ES cells derived from transfected cells that had undergone homologous recombination between the 3′ vector (505) and the endogenous mouse Igκ locus. PCR-positive clones are selected for expansion followed by further analysis using Southern blot assays.

The Southern blot assays are performed according to known procedures; they involve three probes and genomic DNA digested with multiple restriction enzymes chosen so that the combination of probes and digests allowed for conclusions to be drawn about the structure of the targeted locus in the clones and whether it is properly modified by homologous recombination. One of the probes maps to a DNA sequence flanking the 5′ side of the region of identity shared between the 3′ kappa targeting vector (505) and the genomic DNA; a second probe also maps outside the region of identity but on the 3′ side; the third probe maps within the novel DNA between the two arms of genomic identity in the vector, e.g., in the HPRT (545) or neomycin resistance (547) genes. The Southern blot identifies the presence of the expected restriction enzyme-generated fragment of DNA corresponding to the correctly mutated, i.e., by homologous recombination with the 3′ kappa targeting vector (505) part of the kappa locus, as detected by one of the external probes and by the neomycin resistance or HPRT gene probe. The external probe detects the mutant fragment and also a wild-type fragment from the non-mutant copy of the immunoglobulin kappa locus on the homologous chromosome.

Karyotypes of PCR- and Southern blot-positive clones of ES cells are analyzed using an in situ fluorescence hybridization procedure designed to distinguish the most commonly arising chromosomal aberrations that arise in mouse ES cells. Clones with such aberrations are excluded from further use. Karyotypically normal clones that are judged to have the expected correct genomic structure based on the Southern blot data are selected for further use.

Acceptable clones are then modified with the 5′ vector (503) using procedures and screening assays that are essentially identical in design to those used with the 3′ vector (505), except that puromycin selection is used instead of G418/neomycin selection, and the protocols are tailored to match the genomic region modified by the 5′ vector (503). The goal of the 5′ vector (503) transfection experiments is to isolate clones of ES cells that have been mutated in the expected fashion by both the 3′ vector (505) and the 5′ vector (503), i.e., doubly targeted cells carrying both engineered mutations. In these clones, the Cre recombinase causes a recombination (502) to occur between the loxP sites introduced into the kappa locus by the two vectors, resulting in the genomic DNA configuration shown at 507.

Further, the clones must have undergone gene targeting on the same chromosome, as opposed to homologous chromosomes; i.e., the engineered mutations created by the targeting vectors must be in cis on the same DNA strand rather than in trans on separate homologous DNA strands. Clones with the cis arrangement are distinguished from those with the trans arrangement by analytical procedures such as fluorescence in situ hybridization of metaphase spreads using probes that hybridize to the novel DNA present in the two gene targeting vectors (503 and 505) between their arms of genomic identity. The two types of clones can also be distinguished from one another by transfecting them with a vector expressing the Cre recombinase, which deletes the pu-Tk (539), HPRT (545) and neomycin resistance (547) genes if the targeting vectors have been integrated in cis, and comparing the number of colonies that survive ganciclovir selection against the thymidine kinase gene introduced by the 5′ vector (503) and by analyzing the drug resistance phenotype of the surviving clones by a “sibling selection” screening procedure in which some of the cells from the clone are tested for resistance to puromycin or G418/neomycin. Cells with the cis arrangement of mutations are expected to yield approximately 103 more ganciclovir-resistant clones than cells with the trans arrangement. The majority of the resulting cis-derived ganciclovir-resistant clones should also be sensitive to both puromycin and G418/neomycin, in contrast to the trans-derived ganciclovir-resistant clones, which should retain resistance to both drugs. Clones of cells with the cis-arrangement of engineered mutations in the kappa chain locus are selected for further use.

The doubly targeted clones of cells are transiently transfected with a vector expressing the Cre recombinase (502) and the transfected cells are subsequently placed under ganciclovir selection, as in the analytical experiment summarized above. Ganciclovir-resistant clones of cells are isolated and analyzed by PCR and Southern blot for the presence of the expected deletion (507) between the two engineered mutations created by the 5′ vector (503) and the 3′ vector (505). In these clones, the Cre recombinase causes a recombination to occur between the loxP sites (537) introduced into the kappa chain locus by the two vectors. Because the loxP sites are arranged in the same relative orientations in the two vectors, recombination results in excision of a circle of DNA that includes the entire genomic interval between the two loxP sites. The circle does not contain an origin of replication and thus is not replicated during mitosis and is therefore lost from the clones of cells as they undergo clonal expansion. The resulting clones carry a deletion of the DNA that was originally between the two loxP sites. Karyotypes of PCR- and Southern blot-positive clones of ES cells are analyzed using an in situ fluorescence hybridization procedure designed to distinguish the most commonly arising chromosomal aberrations that arise in mouse ES cells. Clones with such aberrations are excluded from further use. Karyotypically normal clones that are judged to have the expected correct genomic structure based on the Southern blot data are selected for further use.

The ES cell clones carrying the deletion of sequence in one of the two homologous copies of their immunoglobulin kappa chain locus are retransfected (504) with a Cre recombinase expression vector and a vector (509) that includes a partly equine immunoglobulin kappa chain locus containing V_{K} (551) and J_{K} (555) gene segments. The key features of the vector are the following: a lox5171 site (531); a neomycin resistance gene open reading frame (547, lacking the initiator methionine codon, but in-frame and contiguous with an uninterrupted open reading frame in the lox5171 site (531) downstream of a methionine start codon (535); a FRT site (527); an array of 19 equine V_{K} gene segments (551), each including equine coding sequences flanked on the 3′ side by mouse RSS and embedded in mouse noncoding sequences; optionally a 13.5 Kb piece of genomic DNA from immediately upstream of the cluster of J kappa region gene segments in the mouse kappa chain locus (not shown); DNA containing the four equine J_{K} region gene segments (555) flanked on the 5′ side by mouse RSS and embedded in mouse noncoding DNA; a loxP site (537) in opposite relative orientation to the lox5171 site (531).

The sequences of the equine V_{K} and J_{K} gene coding regions are shown in SEQ ID NO. 66-86.

The transfected ES clones are placed under G418 selection, which enriches for clones of cells that have undergone RMCE, in which the donor DNA (509) that includes the partly equine immunoglobulin kappa chain locus is integrated in its entirety into the deleted endogenous immunoglobulin kappa chain locus between the lox5171 (531) and loxP (537) sites that were placed there by 5′ (503) and 3′ (505) vectors, respectively. Only cells that have properly undergone RMCE have the capability to express the neomycin resistance gene (547) because the promoter (529) as well as the initiator methionine codon (535) required for its expression are not present in the vector (509) and are already pre-existing in the modified host cell Igκ locus (507). The DNA region created using the 509 sequence is illustrated at 511. The remaining elements from the 5′ vector (503) located between the FRT sites (527) are removed via Flp-mediated recombination (506) in vitro or in vivo, as described below, resulting in the partly-equine immunoglobulin light chain locus as shown at 513.

G418-resistant ES cell clones are analyzed by PCR and Southern blotting to determine if they have undergone the expected RMCE process without unwanted rearrangements or deletions. Karyotypes of PCR- and Southern blot-positive clones of ES cells are analyzed using an in situ fluorescence hybridization procedure designed to distinguish the most commonly arising chromosomal aberrations that arise in mouse ES cells. Clones with such aberrations are excluded from further use. Karyotypically normal clones that are judged to have the expected correct genomic structure based on the Southern blot data are selected for further use.

The ES cell clones carrying the partly equine immunoglobulin kappa chain locus in the endogenous mouse immunoglobulin kappa chain locus (513) are microinjected into mouse blastocysts from strain DBA/2 to create partly ES cell-derived chimeric mice according to standard procedures. Male chimeric mice with the highest levels of ES cell-derived contribution to their coats are selected for mating to female mice. The female mice of choice for use in the mating are of the C57B1/6NTac strain, and also carry a transgene encoding the Flp recombinase that is expressed in their germline and will delete the FRT-flanked neomycin resistance gene (520) and other elements from the 5′ vector. Offspring from these matings are analyzed for the presence of the partly equine immunoglobulin kappa chain locus and for loss of the neomycin resistance gene. Mice that carry the partly equine immunoglobulin kappa chain locus are used to establish colonies of mice.

Mice carrying the partly equine immunoglobulin heavy chain locus, produced as described in Example 1, can be bred with mice carrying a partly equine immunoglobulin kappa chain locus. Their offspring are in turn bred together in a scheme that ultimately produces mice that are homozygous for both the partly equine Igh and the partly equine Igκ. Such mice produce partly equine heavy chains that include equine variable domains and mouse constant domains. They also produce partly equine kappa proteins that include equine kappa variable domains and the mouse kappa constant domain. Monoclonal antibodies recovered from these mice include equine heavy chain variable domains paired with equine kappa variable domains.

In one aspect, the mice that are homozygous for both the partly equine Igh and partly equine Igκ, are bred to mice homozygous for the partly equine lambda loci created in Example 3 to generate mice homozygous for all three loci.

Those skilled in the art will recognize that the 5′ vector (503) and subsequent strategy used here to target the Igκ locus can also be used in place of the 5′ vector (201) in FIG. 2 as an alternate strategy to target the Igh locus. In this case, the 5′ vector (503) is modified to replace the genomic DNA regions (525 and 541) homologous to the Igκ locus with genomic DNA regions (213 and 215 in FIG. 2) homologous to the Igh locus

### Example 3: Introduction of a Heterologous Partly Equine Immunoglobulin Locus into the Immunoglobulin Lambda Chain Gene Locus of a Mouse Genome

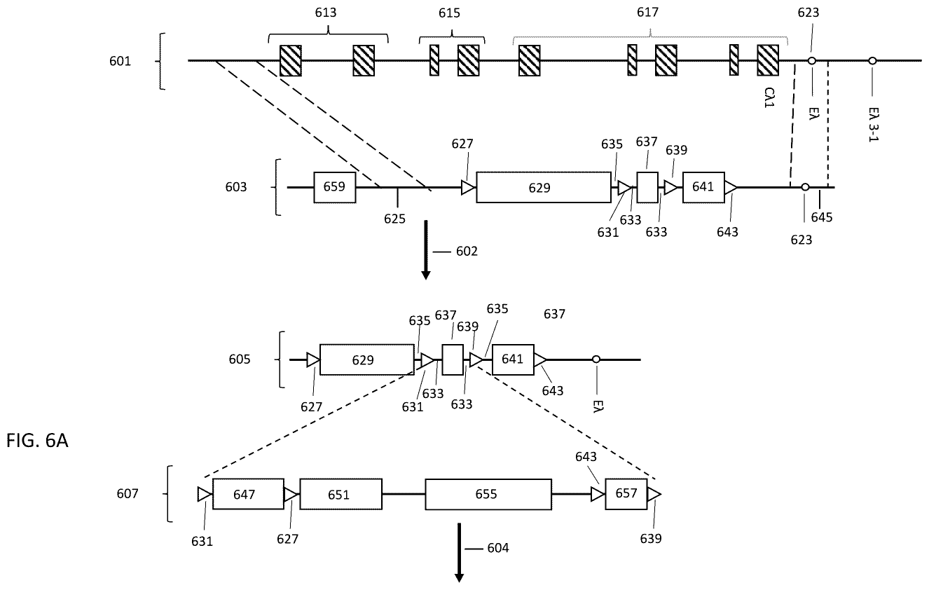

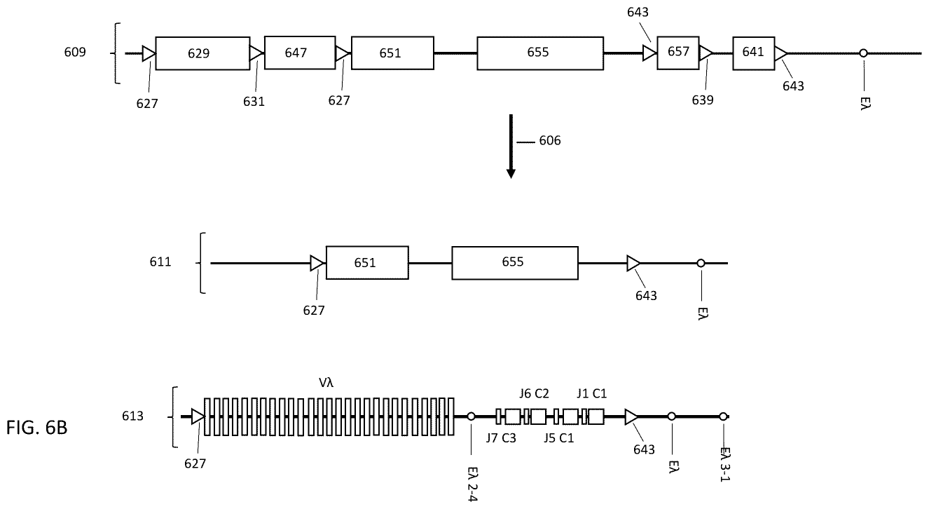

A method for replacing a portion of a mouse Igλ locus with partly equine Igλ locus is illustrated in FIGS. 6A and 6B. This method includes deleting approximately ˜200 Kb of DNA from the wild-type mouse immunoglobulin lambda locus (601 and FIG. 1, bottom) that includes Vλ2/Vλ3 gene segments (613), Jλ2/Cλ2 gene cluster (615), and Vλ1-Jλ3/Cλ3-Jλ1/Cλ1 gene cluster (617) by a homologous recombination process involving a targeting vector (603) that shares identity with the endogenous mouse immunoglobulin lambda locus both upstream of the Vλ2/Vλ3 gene segments (613) and downstream of the Cλ1 gene segment (rightmost box in 617) and either upstream or downstream of the EX enhancer (623). The vector replaces the ˜200 Kb of the endogenous mouse genomic DNA with elements designed to permit a subsequent site-specific recombination in which a heterologous immunoglobulin lambda locus replaces the modified VA locus via RMCE (604). In this example, the heterologous immunoglobulin lambda locus is a synthetic nucleic acid that includes equine Igh coding sequences and mouse Igλ non-coding sequences.

The key features of the gene targeting vector (603) for accomplishing the ˜200 Kb deletion and inserting the site-specific recombination sites are as follows: a negative selection gene such as a gene encoding the A subunit of the diphtheria toxin (DTA, 659) or a herpes simplex virus thymidine kinase gene (not shown); 4 Kb of genomic DNA from 5′ of the mouse Vλ2/Vλ3 variable region gene segments in the immunoglobulin lambda locus (625); a FRT site (627); genomic DNA containing the mouse Polr2a gene promoter (629); a translation initiation sequence (methionine codon embedded in a “Kozak” consensus sequence) (635); a mutated loxP recognition sequence (lox5171) for the Cre recombinase (631); a transcription termination/polyadenylation sequence (633); an open reading frame encoding a protein that confers resistance to puromycin (637), whereas this open reading frame is on the antisense strand relative to the Polr2a promoter and the translation initiation sequence next to it and is followed by its own transcription termination/polyadenylation sequence (633); a loxP recognition sequence for the Cre recombinase (639); a translation initiation sequence (a methionine codon embedded in a “Kozak” consensus sequence) (635) on the same antisense strand as the puromycin resistance gene open reading frame; a chicken beta actin promoter and cytomegalovirus early enhancer element (641) oriented such that it directs transcription of the puromycin resistance open reading frame, with translation initiating at the initiation codon downstream of the loxP site (635) and continuing back through the loxP site into the puromycin open reading frame all on the antisense strand relative to the Polr2a promoter and the translation initiation sequence next to it; a mutated recognition site for the Flp recombinase (643); and genomic DNA (645) containing the EX enhancer element (623).

Mouse embryonic stem (ES) cells derived from C57B1/6NTac mice are transfected (602) by electroporation with the targeting vector (603) according to known procedures. Homologous recombination replaces the endogenous mouse immunoglobulin lambda locus with the site-specific recombination sites from the targeting vector (603) in the ˜200 Kb region resulting in the genomic DNA configuration depicted at (605).

Prior to electroporation, the vector DNA is linearized with a rare-cutting restriction enzyme that cuts only in the prokaryotic plasmid sequence or the polylinker associated with it. The transfected cells are plated and after ˜24 hours placed under positive drug selection using puromycin. There is also negative selection for cells that have integrated the vector into their DNA but not by homologous recombination. Non-homologous recombination will result in retention of the DTA gene (659), which will kill the cells when the gene is expressed, whereas the DTA gene is deleted by homologous recombination since it lie outside of the region of vector homology with the mouse Igλ locus. Colonies of drug-resistant ES cells are physically extracted from their plates after they become visible to the naked eye over a week later. These picked colonies are disaggregated, re-plated at limiting dilution in micro-well plates and cultured for several days. Thereafter, each of the clones of cells are divided such that some of the cells are frozen as an archive, and the rest used for isolation of DNA for analytical purposes.

DNA from the ES cell clones is screened by PCR using a known gene-targeting assay. For these assays, one of the PCR oligonucleotide primer sequences maps outside the regions of identity shared between the targeting vector and the genomic DNA, while the other maps within the novel DNA between the two arms of genomic identity in the vector, e.g., in the puro gene (637). These assays detect pieces of DNA that would only be present in clones of cells derived from transfected cells that had undergone homologous recombination between the targeting vector (603) and the endogenous DNA (601).

PCR-positive clones from the transfection are selected for expansion followed by further analysis using Southern blot assays. The Southern blots involve three probes and genomic DNA from the clones that has been digested with multiple restriction enzymes chosen so that the combination of probes and digests allow identification of whether the ES cell DNA has been properly modified by homologous recombination.

Karyotypes of the PCR- and Southern blot-positive clones of ES cells are analyzed using an in situ fluorescence hybridization procedure designed to distinguish the most commonly arising chromosomal aberrations that arise in mouse ES cells. Clones that show evidence of aberrations are excluded from further use. Karyotypically normal clones that are judged to have the expected correct genomic structure based on the Southern blot data are selected for further use.

The ES cell clones carrying the deletion in one of the two homologous copies of their immunoglobulin lambda chain locus are retransfected (604) with a Cre recombinase expression vector together with a vector (607) that includes a partly equine immunoglobulin lambda chain locus containing equine Vλ and JA region gene segment coding sequences. The key features of this vector (607) are as follows: a lox5171 site (631); a neomycin resistance gene open reading frame lacking the initiator methionine codon (647), but in-frame and contiguous with an uninterrupted open reading frame in the lox5171 site (631 in diagram 605)); a FRT site (627); an array of 27 functional equine lambda variable region gene segments, each gene segment including equine lambda coding sequences flanked on the 3′ side by mouse RSS and embedded in mouse lambda noncoding sequences (651); an array of J-C units where each unit includes an equine JA gene segment and a mouse lambda constant domain gene segment embedded within noncoding sequences from the mouse lambda locus (655), including the Eλ2-4 enhancer element (FIG. 1). The equine Jλ gene segments are those encoding Jλ1, Jλ5, Jλ6 and Jλ7 (the other Jλ gene segments are pseudogenes) while the mouse lambda constant domain gene segments are Cλ1, Cλ2 or Cλ3 or a combination thereof; a mutated recognition site for the Flp recombinase (643); an open reading frame conferring hygromycin resistance (657), which is located on the antisense strand relative to the immunoglobulin gene segment coding information in the construct; a loxP site (639) in opposite relative orientation to the lox5171 site.

RCME inserts the partly equine immunoglobulin lambda chain locus from the RCME vector (607) into the modified endogenous mouse Igλ locus resulting in the genomic DNA configuration depicted at 609.

The sequences of the equine Vλ and JA gene coding regions are shown in SEQ ID NO. 87-122.

The transfected clones are placed under G418 or hygromycin selection, which enriches for clones of cells that have undergone a RMCE process, in which the partly equine immunoglobulin lambda chain variable is integrated into the deleted endogenous mouse immunoglobulin lambda chain locus between the lox5171 and loxP sites that were placed there by the gene targeting vector. The remaining elements from the targeting vector (603) are removed via FLP-mediated recombination (606) in vitro or in vivo (see below) resulting in the final partly equine immunoglobulin lambda chain locus as shown at 611.

A more detailed view of one configuration of the 611 partly equine immunoglobulin lambda chain locus is shown at 613 but is only provided as an example. Other arrangements and numbers of equine Vλ and JA gene segments and murine CA gene segments, as well as the position and number of enhancer elements are also possible.

G418/hygromycin-resistant ES cell clones are analyzed by PCR and Southern blotting to determine if they have undergone the expected recombinase-mediated cassette exchange process without unwanted rearrangements or deletions. Karyotypes of the PCR- and Southern blot-positive clones of ES cells are analyzed using an in situ fluorescence hybridization procedure designed to distinguish the most commonly arising chromosomal aberrations that arise in mouse ES cells. Clones that show evidence of aberrations are excluded from further use. Karyotypically normal clones that are judged to have the expected correct genomic structure based on the Southern blot data are selected for further use.

The ES cell clones carrying the partly equine immunoglobulin lambda chain locus (611) in the mouse immunoglobulin lambda chain locus are microinjected into mouse blastocysts from strain DBA/2 to create partially ES cell-derived chimeric mice according to known procedures. Male chimeric mice with the highest levels of ES cell-derived contribution to their coats are selected for mating to female mice. The female mice of choice here are of the C57B1/6NTac strain, which carry a transgene encoding the Flp recombinase expressed in their germline will delete the FRT-flanked selectable markers. Offspring from these matings are analyzed for the presence of the partly equine immunoglobulin lambda chain locus, and for loss of the FRT-flanked neomycin resistance gene and the mFRT-flanked hygromycin resistance gene that were created in the RMCE step. Mice that carry the partly equine immunoglobulin lambda chain locus are used to establish a colony of mice.

In one aspect, the mice homozygous for the partly equine immunoglobulin heavy chain locus and the partly equine immunoglobulin kappa light chain locus (as described in Examples 1 and 2) are bred to mice that carry the partly equine immunoglobulin lambda light chain locus. Mice generated from this type of breeding scheme are homozygous for the partly equine Igh locus and homozygous for the partly equine Igκ and IgA loci. Monoclonal antibodies recovered from these mice include equine heavy chain variable domains paired in some cases with equine kappa variable domains and in other cases with equine lambda variable domains.

TABLE 1
The Vλ gene segments in cluster II are expressed as cDNAs

| | | Corresponding |
|---|---|---|
| Vλ gene | Vλ gene | GenBank |
| designation¹ | designation² | cDNA |
| Vλ21 | IGLVxS64 | KF985057.1 |
| Vλ22 | IGLV8S5 | KF985111.1 |
| Vλ23 | IGLV8S6 | KF748660 |
| Vλ24 | IGLV9S2 | XM_014835914³ |
| Vλ25 | IGLV8S7 | KF985059.1 |
| Vλ26 | IGLV8S8 | KF985065.1 |
| Vλ27 | IGLV8S9 | KF985065 |

¹Nomenclature of Sun, et al. Dev. Comp. Immunol. 34:1009 (2010)
²Nomenclature of Walther, et al. Dev. Comp. Immunol. 53:303 (2015)
³This cDNA is from E. asinus, the others are from E. caballus

SEQUENCE INFORMATION
Note:
(RC) stands for reverse complement,
indicating that the sequences are in the
opposite transcriptional orientation
compared to the other sequences.
IGHV
SEQ ID NO. 1:
VH1 IGHV1-5*01
L1:
ATGGACTGGAGCTGGAGCATCCTCTTCTTGGTGGCAGTGGCTGCAG
L2:
GTGTCTCCTCC
VH:
GAGGTCCAGCTGGTGCAGTCTGGGGCTGAGGTGAAGAAGCCAGGG
GCATCCGTGAAGGTCTCCTGCAAGGCTTCTGGAGACAGCTTCACT
TATTACTCTATGAGCTGGGTGCGACAGGCCCCTGGACAAGGGCTT
GAGTGGACGGGATATATCTATCCTGAATATGATGCTATGGGCTAC
CCGCAGAAGTTCCAGGGCAGAGTCACCATGACTGCGGACAAGTCC
ACGAGCACAGTCTACATGGAGCTGAGCAGTCTGGCATCTGAGGAC
ACAGCCGTGTATTACTGTGCAACAGA
SEQ ID NO. 2:
VH2 IGHV4-11*01
L1:
ATGAATCACCTGTGGTTCTTCCTCTTTCTGGTGGCCGCTCCTGCAT
L2:
GTGTCCTGTCC
VH:
CAGGTGCAACTGAAGGAGTCAGGACCTGGCCTGGTGAAGCCCTCG
CAGACCCTGTCCCTCACCTGCCCTGTCTCTAGATTCCCTTTAACC
AACCATCATGTACACTGGACCCACCAGGCTCCAGGAAAAGGGCTG
GAGTGGCTTGGTGATTCAAGGAGTGGTGAAAGCACATACTACAAC
TTAACTCTGAAGTCCCAACTCAGCATCCCCAGTGATACTTCCAAA
AGCCAAATTTATTTAACGCTGAACAGGCTGAGAGGCGATGACATG
GCCATGTACTACTGTGCCAGAGA
SEQ ID NO. 3:
VH3 IGHV4-17*02
L1:
ATGAGACTCTTGTGTCTTCTCCTTTGCCTGGTGATGGCTCCCCAAG
L2:
GAGTCCTGTCC
VH:
CAGGTGAAGCTGCAGGAGTCGGGCCCAGGACTGGTGAAGCCCTCA
CAGACCCTCTCCCTCACCTGCTCTGTGTCTGGAGTCTCCATCACA
AGCAGTGGTGACTGGTGGAGCTGGATCCGCCAGCCCCCAGGGAAG
GGGCTGGAATGGATGGGGTACATAAGTTATAGTGGTAGCGCTTAC
TACACCACATCCCTCAAGAGCCGACTCTCCATCTCCAGAGACACG
TCCAAGGACCAGTTCTCCCTGCAGCTGAGCTCCGTGACCACAGAG
GACACGGCCGTTTATTACTGTGCAAGTGA
SEQ ID NO. 4:
VH4 IGHV4S1
L1:
ATGAATCACCTGTGGTTCTTCCTCTTTCTGGTGGCCGCTCCTACAT
L2:
GTGTCCTGTCC
VH:
CAGGTGCAACTGAAGGAGTCGGGACCTGGCCTGGTGAAGCCCTCG
CAGACCCTGTCCCTCACCTGCACTGTCTCTGGATTATCTTTGAGC
AGTAATGCTGTAGGCTGGGTCCGCCAGGCTCCAGGAAAAGGGCTG
GAGTGGGTTGGTGTTATATATGGTAGTGAAAGTACATACTACAAC
CCAGCCCTGAAGTCCCGAGCCAGCATCACCAAGGACACCTCAAAG
AGCCAAGTTTATCTGACGCTGAACAGCCTGACAGGCGAAGACACG
GCCGTCTATTACTGTGCAGGATG
SEQ ID NO. 5:
VH5 IGHV4-29*02
L1:
ATGAGTCACCTGTGGTTCTTCCTCTTTCTGGTGGCCGCTCCTACAT
L2:
GTGTCCTGTCC
VH:
CAGGTGCAACTGAAGGAGTCAGGACCTGGCCTGGTGAAGCCCTCG
CAGACCCTGTCCCTCACCTGCACTGTCTCTGGATTATCTTTGAGC
AGTTATGCTGTAGGCTGGGTCCGCCAGGCTCCAGGAAAAGGGCTG
GAATATGTTGGTGCTATATATGGTAGTGCAAGTGCAAACTACAAC
CCAGCCCTGAAGTCCCGAGCCAGCATCACCAAGGACACCTCAAAG
AGCCAAGTTTATCTGACGCTGAACAGCCTGACAGGCGAGGACACG
GCCGTCTATTACTGTGCGAGA
SEQ ID NO. 6:
VH6 IGHV1-41*01
L1:
ATGGACTGGAGCTGGAGCATCCTCTTCTTGGTGGCAGTGGCTGCAG
L2:
GTGTCTCCTCC
VH:
GAGGTCCAGCTGGTGCAGTCTGGGGCTGAGGTGAGGAAGCCAGGG
GCATCCGTGAAGGTCTCCTGCAAGGCTTCTGGAGACAGCTTCACT
TATTACTCTATGAGCTGGGTGCGACAGGCCCCTGGACAAGGGCTC
GACTGGATGGGAGGGATCTTGCCTATAGTTGATGATACAAGCTAC
ACGCAGAAGTTCCAGGGCAGAGTCACCATGACTGCAGACAAGTCC
ACGAGCACAGTCTACATGGAGCTGAGCAGTCTGACATCCGAGGAC
ACGGCCGTGTATTACTGTGCAAAAG
A
SEQ ID NO. 7:
VH7 IGHV1-70*01
L1:
ATGGGCTGGAGCTGGAGAATCCTCTTCTTGGTGGCAGTAGCTTCAG
L2:
GTGTCTCCTCC
VH:
GAGGGTCAGCTGGAACAGTCGGGGCCGGAGTTGAAGAAGCCTGGG
TCATCAGTGAAGATCTCCTGCAAGGCTTCTGGATACACCTTCAGT
AGCTATGCTGTGCACTGGGTGCGACAGGCCAATGGAAAAGGGATT
GAGTGGATGGGATCTATCTATGCTGAATATGATGATACAAGCTAC
GCACCGAAGTTCCAGGGCAGAGTCACCATGACTGCGGACAAGTCC
ACGAGCACAGTCTACATGGAGCTGAGCAGTCTGACATCTGAGGAC
ATGGCCGTGTATTACTGTGCAACAG
A
SEQ ID NO. 8:
VH8 IGHV9-66*01
L1:
ATGGCCCCTCTCCTGGTCATCTTCTGCCTGCTGGCTGCTCTCCACG
L2:
GTGTCGAGGCT
VH:
GAGGACCCTCTCGTGCAATGGGGAGGTGGAGTGGTGGTCTCCTCA
CAGACACTCAGCCTCACCTGTGCCGCCTACAAACGCAAAGTTTCA
GAATATTCCCTGTGGTGGATTCGCCTTCTCCCAGGGAAGGGGTTG
GAGTGCGTAGGTGTGATCTGGGCTAAGGGGGACACTCAGTGCAGC
CCCCACCTGCAGTCTCGAGTCAGCATCTCCAGGGACGCCACCAAG
AACCAAGTGTTCTTACAGCTGAGCAGTGTGATGCCTGAGGATTCA
GGCGTGTATTACTGTGCTCAAGA
SEQ ID NO. 9:
VH9 IGHV4-65*02
L1:
ATGAGACTCTTGTGTCTTCTCCTTTTCCTGGTGACGGCTCCCCAAG
L2:
GAGTCCTGTCC
VH:
CAGGTGCAGCTGCAGGAGTCGGGCCCAGGACTGGTGCAGCCCTCA
CAGACCCTGTCCCTCACCTGCACTGTCACTGGAGGCTCCATCACA
AGCAGCTATTCTAGCTGGAGCTGGTTACGCCAGCCTCCAGGGAAG
GGGCTGGAGTACATGGGGTACATATATTATGATGGTAGAACTTAC
TACAATCCTTCCTTCAAGAGCCGCACCTCCATCTCCAGAGACACC
TCCAGGAACCAGTTCTCCCTGCAGCTGAGCTCCGTGACCACCGAG
GACGCGGCCGTGTATTACTGTGCAAGAGA
SEQ ID NO. 10:
VH10 IGHV2-63*01
L1:
ATGGACACACTGTATCCCACCCTCCTGCTGCTGACCATCCCTTCCT
L2:
GGGCTCTGTCC
VH:
CAGATCAGCCTGCAGGAGTCTGGTCCTGGGCTGCTGAAGCCCACC
CAGACCCTTACGCTGACCTGCTCCTTCTCTGGGTTCTCACTGACT
ACTTCTGATATTGGTGTTGGTTGGATGCGTCAACCCCCTGGGAAG
GCACTGGAGTGGCTCACCTATGTTTGGTGGACTGATGAAAAGCAT
TACAACCCATCTCTGAAGAGCCGGCTCACAATCTCCAAGGACACC
TCCAAAAACCAGGTGATGCTGACAATGACCAGTTTGGACCCTCCA
GACACAGCCACATATTACTGTGTAAAGAGGG
SEQ ID NO. 11:
VH11 IGHV4-59
L1:
ATGAGGAGGCTGGGTCTTCTCCTTTTCCTGGTGACGGCTCCCCAAG
L2:
GTGTCCTCTCC
VH:
CAGGTGCAGCTGCAGGAGTCAGGACCAGGCCAGACGAATCCCTCA
CAGACCCTGTCCCTCACATGCACTGTCACTGGTTACTCCATCACC
AGTGGTTATGGCTGGAACTGGATCCGCCAGCCACCAAACAAAGGG
CTGGAGTGGATGGGGAGCATAAGCTATAGTGGTAGAACTAACTAC
AGCCCATCCCTCAGGAGCCGCATCACCATCTCCAGAGACACTTCC
AAGAACCAGTTCTTGCTGCAGCTGAGCTCAGTAACCACTGAGGAC
ACGGCCGTGTATTACTGTGCGACAGA
SEQ ID NO. 12:
VH12 IGHV4-55
L1:
ATGAGGCTGTTGGGTCTTCTCCTTTGTCTTGTGACGGCTTACCAGG
L2:
GTGTCCTGTCC
VH:
CAGGTGCAGCTGCAGGAGTCGGGCCCAGGACTGGTGAAGCCCTCA
CAGACCCTCTCCCTCACCTGCACTGTCACTGGTTACTCCATCACC
AGTGGTTACTACTGGAGCTGGATCCGTCAGCCCCCAGGAAAGAGG
CTGGAGTGGATGGGCTCCATATATTATAGTGGTAGCACTTACTAC
AGCCCATCCCTCAAGAGCCGCATCACCATCTCCACAGACACGTCC
CAGAACCAGTCCTCCCTGCAGCTGAGCTCCGTGACCACCAAGGAC
ACAGCTGTGTATTACTGTGCCAGAGA
IGHD
SEQ ID NO. 13:
DH1 IGHD3-1
GTATTATTCTCCTGGATAGAGTAATTACAAC
SEQ ID NO. 14:
DH2 IGHD4-2
TTACTATGGCTGGGGTAAC
SEQ ID NO. 15:
DH3 IGHD2-3
ATGATGACTATGGTGATACTTTCTACTATACA
SEQ ID NO. 16:
DH4 IGHD3-4
GTATTACTCTTCTGCATATGATTGTATCAAC
SEQ ID NO. 17:
DH5 IGHD4-5
TTACTACAACTATAACTAC
SEQ ID NO. 18:
DH6 IGHD2-6
ATGGTTACTATAGTAGGAGTTGCTATACC
SEQ ID NO. 19:
DH7 IGHD3-7
GGATTTACTGTTCTGGGTGCAGATGCTCGTTACAACCACAGCAA
SEQ ID NO. 20:
DH8 IGHD4-8
TAACTACAGATATAGCTCC
SEQ ID NO. 21:
DH9 IGHD2-9
ATGGTTACTATGCTAGTGGTTATGACTACA
SEQ ID NO. 22:
DH10 IGHD3-10
GTATTACTCTTCTGCATATGCTTGTATCAAC
SEQ ID NO. 23:
DH11 IGHD4-11
TAACTACGGTTATGGTTATGCTAC
SEQ ID NO. 24:
DH12 IGHD2-12
ACTATAGTTATGGTAGTTACTATGCC
SEQ ID NO. 25:
DH13 IGHD3-13
GTATGACTGTACTGGTCATGGATGTGTCTACATC
SEQ ID NO. 26:
DH14 IGHD4-14
TAACTACTATGGTAGCAAC
SEQ ID NO. 27:
DH15 IGHD2-15
ATGGTTACTATGGTAGTTACTACAGTAGTTACTATGCC
SEQ ID NO. 28:
DH16 IGHD3-16
GTATTACTATTCTGGATATAATTATTACAAC
SEQ ID NO. 29:
DH17 IGHD4-17
GCCACTGATATAGCTCC
SEQ ID NO. 30:
DH18 IGHD2-18
ATGGTTACTATGCTGGTAGTTACTATGCC
SEQ ID NO. 31:
DH19 IGHD3-19
GTGTGAATGTCCTGGGCATGGATGTTATTACGAC
SEQ ID NO. 32:
DH20 IGHD4-20
TTCCTACCGATATAGCTCC
SEQ ID NO. 33:
DH21 IGHD2-21
ACGGTTCCTATGCTGGTAGTTACTTATACTACA
SEQ ID NO. 34:
DH22 IGHD3-22
GTATTACTATTCTGCATATGATTATTACAAC
SEQ ID NO. 35:
DH23 IGHD2-23
ATGATTACTATGGTATTAGTGACTCCTACA
SEQ ID NO. 36:
DH24 IGHD2-24
GTATTACTCTTTTGAATATGGTTATAACAAC
SEQ ID NO. 37:
DH25 IGHD4-25
CTGCTATAGCAGCTATGCTTACTAC
SEQ ID NO. 38:
DH26 IGHD2-26
ACTATGGTTATGGTGGTGCTTACTACTACA
SEQ ID NO. 39:
DH27 IGHD3-27
GTATTACTATTCTGCATTTCGTTATTACAAC
SEQ ID NO. 40:
DH28 IGHD4-28
CTGCTATAGCAGCTATGCTTACTAC
SEQ ID NO. 41:
DH29 IGHD2-29
ACAGTTACTATGGTGGTAGTTCCTGGTACTCC
SEQ ID NO. 42:
DH30 IGHD3-30
GTATTACTATTCTGGACATGATTATTACAACCTCAGCGT
SEQ ID NO. 43:
DH31 IGHD4-31
TTACGATGACGGATACTACAAC
SEQ ID NO. 44:
DH32 IGHD1-32
GGTCCTGGGTACAGCTCC
SEQ ID NO. 45:
DH33 IGHD7-33
AGATACTCCAGTGCTGGTTAC
SEQ ID NO. 46:
DH34 IGHD6-34
CTACGGTAGCGGTTGGCC
SEQ ID NO. 47:
DH35 IGHD4-35
TAACTATGGCTCCTATAATTACTAC
SEQ ID NO. 48:
DH36 IGHD2-36
ATGATTATTATGGTGCTATTGACTACATAAC
SEQ ID NO. 49:
DH37 IGHD3-37
TATGACAATTCTGTATATAGCTCTGACTACAGCAT
SEQ ID NO. 50:
DH38 IGHD4-38
GGAGAAGAGTTGGAGTAAC
SEQ ID NO. 51:
DH39 IGHD2-39
ACAGTTACTGGAGTAGTAGTTACTATGCC
SEQ ID NO. 52:
DH40 IGHD3-40
GAATAACTATGCTACATATGATTATATCAAC
SEQ ID NO. 53:
DH41 IGHD4-41
AACTGCTATGGTAACAAC
SEQ ID NO. 54:
DH42 IGHD1-42
GGTACTTGGGTACAGCTCC
SEQ ID NO. 55:
DH41 IGHD7-43
AGATACTCCAGTGTTGGTTAC
SEQ ID NO. 56:
DH41 IGHD6-44
ATACGGTAGTGGTTGGCC
IGHJ
SEQ ID NO. 57:
JH1 IGHJ1
CTTATGCTTACTTGCAGCACTGGGGCCA
CGGCACCCTGGTCACCGTCTCCTCAG
SEQ ID NO. 58:
JH2 IGHJ2
GTCCTGGCACCTCGAGCACTGGGACCACG
GCATCCTGGTCACCGTCTCCTCAG
SEQ ID NO. 59:
JH3 IGHJ3
GTTATGGCTACGTGGATCACTGGGGCCAG
GGCACCCTGGTCACCGTCTCCTCAG
SEQ ID NO. 60:
JH4 IGHJ4
ACTATTTTGGCTACTGGGGCCAGGGCACC
CTGGTCACCGTCTCCTCAG
SEQ ID NO. 61:
JH5 IGHJ5
ACAACGAGTTGGATTACTGGGGCCAGGGCA
CCCTGGTCACCGTCTCCTCAG
SEQ ID NO. 62:
JH6 IGHJ6
ATTATTATGGTATAAACTACTGGGGCCAGGG
CATCCTGGTCACCGTCTCCTCAG
SEQ ID NO. 63:
JH7 IGHJ6-2
ATTATTATAATGCTATGGACCCCTGGGGCCA
GGGCACCCTGGTCACCGTCTCCTCAG
SEQ ID NO. 64:
JH8 IGHJ6-3*
ATTATTATGATATAGACTACTGGGGCCAGGG
CACCCTGGTCACCGTCTCCTCAG
SEQ ID NO. 65:
JH9 IGHJ6-4*
ATTATTATGATATAGACTACTGGGGCCAGGG
CACCCTGGTCACCGTCTCCTCAG
*JH8 and JH9 are separate gene segments
but have identical sequence.
IGKV
SEQ ID NO. 66:
IGKV2-48*01
L1:
ATGAGGTTCTCTGCTCAGCTCCTGGGGTT
GCTAATACTCTGGGTCCCAG
L2:
GATCCACTGGG
VK:
GACACAGTTTTGACCCAGACCCCACTCTCTCTGTCTGTCATCCCT
GGAGAGTCGGCCTCCATCTCTTGCAAGTCTAGTCAGAGCCTCCTA
CATGGTAATGGAAACACCTATTTGCATTGGTACCTGCAGAAGCCA
GGCCAGTCTCTTCAGCGCCTGATCTCTATGGTTTCCAATCGGGCA
TCTGGGGTCCCAGACAGGTTCAGTGGCAGCGGGTCTGGGACAGAT
TTCACCCTTATAATCAGCAAGTTGGAAGCTGAGGATGTTGGAGTT
TATTACTGCATGCAAGCTACACAAAGTCCCCC
SEQ ID NO. 67:
IGKV2-46*01
L1:
ATGAAATTCGCTAGTCAGCTCCTGGGGCTACTGATGCTCTGGATCCCAG
L2:
GATCCAGTGCG
VK:
GATGTTGTGTTGACCCAGACTCCACTCTCCCTGTCTGTCGTCCCT
GGAGAGCCGGCCTCCATCTCCTGCAAGTCTAGTCAGAGCCTCAAA
TATAGTGATGGGAAAACCTATTTGTATTGGTTCCTACAGAAGCCA
GGCCAGTCTCCAAAGCTCCTGATCTATTTGGTTTCCACCCGGTAC
TCTGGGGTCTCAGACAGGTTCAGTGGCAGCGGATCAGAAGCAGAT
TTCACCCTGAAAATCAGCAGAGTGGAGCCTGAGGATGTTGGAGTC
TATTACTGCTTTCAAGCTCTATATGCTTCTCC
SEQ ID NO. 68:
IGKV2-45*01
L1:
ATGAGGCTCCCTGCTCAGCTCCTGGGGCTGCTGATGCTCTGGATCACAG
L2:
GATCCAGTGGG
VK:
GATGTTGTGATGACCCAGACTCCACTCTCCCTGTCTGTCGTCCCT
GGAGAGCCGGCCTCCATCTCCTGCAAGTCTAGTCAGAGCCTCAAA
CATAGTGATGGAAAAACCTATTTGTATTGGTTCCTACAGAAGCCA
GGCCAGTCTCCAAAGTGCTTGATCTATTTGGTTTCCACCCGGGTC
TCTGGAGTCTCAGACAGGTTCAGTGGGAGCGGGTCAGAAACAGAT
TTCACCCTGAAAATCAGCAGAGGGGAGCCTGAGGACGTTGGAGTC
TATTACTGTGTGCAAGCTCTATATGCTTCTCC
SEQ ID NO. 69:
IGKV5-43*01
L1:
ATGGGCTCCCAGGCTCAGCTCCTCAGCTTCCTGCTCCTCTGGATTTTTG
L2:
ATACCAGGGCA
VK:
GAAATAACAGTCACACAGTCTCCGGAATCCATGTTAGTGATTCCA
GGAGACAAAGTCATCATCACCTGCAAAGCCAGCCAAGACATTGGT
GATGATGTGAACTGGTATCAATGGAAACCAGGAGAAGCTCCTAAG
CTCATTATTAAAGAAGCTACTACTCTCTGGTCTGGGGTTCCCTCT
CGGTTCAGTGGCACTGTGCATGGAGTAGATTTTACCCTGACAATT
GAGGACGTAAAATCTGAGGATGCTGCATATTATTTCTGTCTACAA
CATGATCGTATACCTCT
SEQ ID NO. 70:
IGKV2-39*01
L1:
ATGAGGCTCCCTGCTCAGCTCCTGGGGCTGCTGATGCTCTGGATCCCAG
L2:
GATCCAGTGGG
VK:
GATGTTGTGATGACCCAGACTCCACTCTCCCTGCCTGTCGTCCCT
GGAGAGCCGGCCTCCATCTCCTGCAAGTCTAGTCAGAGCCTGCTG
GATAGTGATGGAAAAACCTATTTGTATTGGTACCTGCAGAAGCCG
GGCCAGTCTCCAAAGCTCCTGATCTATTCAGTTTCCAACCGGGAC
TCTGGGATCTCAGACAGGTTCAGTGGCAGCGGGTCAGGAACAGAT
TTCACCCTGAAAATCAGCAGAGTGGAGCCTGAGGATGTTGAAGTC
TATTACTGTGTGCAAGCTACACATGCTCCTCC
SEQ ID NO. 71:
IGKV1-36*01
L1:
ATGAGGGTCCCTGCTCAGCTCCTCAGCCTTCTGCTGCTCTGGCTCCCAG
L2:
GTGCCAGGTGT
VK:
GAGATCCAGATGACCCAGTCTCCAGCCTCCCTGTCTGCATCTCTA
GGAGACAGAGTCACCATCACTTGCCAGGCCACTCAGGGCATTAAC
ACTTGGTTAGCCTGGTATCAGCAGAAACCAGGGAAAGCTCTTAAG
TTCCTGATCAGTAAGGCAACCATTTTGCACACTGGCGTCTCTTCG
AGGTTCAGTGGCAGTGGAACTTGGACAGATTTCACTCTCACCATC
AGCAGCCTGGAGCCTGAAGATGCTGCAACTTATTACTGTCAGCAG
TATAAGAGCAGCCCTCC
SEQ ID NO. 72:
IGKV2-33*01
L1:
ATGAAATTCCTTGCTCAGCTCCTGGGGCTGCTAATGCTCTGGATCCCAG
L2:
GATCCAGTGGA
VK:
GATATTGTGATGACCCAGACTCCACTCTCCCTGCCTGTCGTCCCT
GGAGAGCTGGCCTCCAACTCATGCAGGTTTAGTCAGAGCCTCCTA
CATAGTAATGGAAACACCTATTTGCACTGGTTCCTGCAGAAGCCA
GGCCAATCTCCAAGGCGTCTGACCTATAGGGTGTCCAACCGGAAC
TCTGGGGTCCCAGACAGGTTCATTGGCAGCGGGTCAGGGACAGAT
TTTACACTTAAAATCAGCAAGGTGGAGGCTGAAGATGGTGGAGTT
TATTATTGCTCCCAAGGTACACAAAGTCCCCC
SEQ ID NO. 73:
IGKV2-28*01
L1:
ATGAGGTTCCCTGCTCAGCTCCTGGGGCTACTAATGCTCTGGATCCCAG
L2:
GATCCAGTGGA
VK:
GATATTGTGATGACCCAGACTCCCCTCTGCTTGGCCGTCACCTTG
GGAGAGCCAGTTTCCATCTCCTGCAGGTCTAGTCAGAGCCTCCTC
CGTAGTGATGACTACACCTATTTGGATTGGTACCTGCAGAAACCA
GGCCAGTCTCCACGGCTGCTGATCTATGAGGTTTCCAAGCTGGTC
TCTGGAGTCTCAGACAGGTTCAGTGGCAGTGGGTCAGGGACAGAT
TTCACCCTTCAAATCAGCAGAGTGGAGGCTGAGGATGTTGGAGTT
TATTACTGCATGCAAGGTTCACAAAGACCTCC
SEQ ID NO. 74:
IGKV4-18*01 (RC)
L1:
ATGATGTCACTGACAAAGGTGTTTATGTCTTTGTTGCTCTGGGTCTCAA
L2:
CTGCCTGTGGG
VK:
GACATCGTGATGACCCAGTCTCCAGGCTCCTTGGCAGTGTCTCCA
GGACAGAGGGTCACCATTAGCTGCAAGGCCAGTCAGAGTGTTAGC
AACTACTTAGACTGGTACCAGCAAAAACCAGGAGAGGCTCCTATG
CTGCTTATCTATGCGGCATCCAGCAGAGCATCTGGGGTCCCCGAC
AGATTCAGTGGCGGTGGATCTGGGACAGATTTCGCTCTCACCATC
AGCAGCCTCCAGGCTGAAGATGTGGCAGTTTTCACTGTCAGCAGC
ATTATACTAATCCTCCC
SEQ ID NO. 75:
IGKV4-12*01 (RC)
L1:
ATGATGTGGGAGACACAGGTCCTTATGTCCTTATTGCTCGGGGTCTCAG
L2:
GTACCTTGGGG
VK:
GACATCATGATGACCCAGTCTCCAGACTCCTTGGCAGTGTCTCTA
GGAGAGAGGGTCGACATGAAGTGCACGGCCAGTCAGAGTGTTTAC
CACTACTTAGCCTGGTACCAGCAAAAACCAGGACAGGCTCCTAAG
CCCCTCATCTACTCAGCATCTACCAGACCATCTGGGATCCCTGAC
CGATTCAGTGGCAGTGGATCTGGGACAGATTTCACTCTCACCATC
AGCAACTTCCAAGCTGAAGATGCGGCAGTTTATTGCTGTGAGCAG
TATTATGGTAATCCTCC
SEQ ID NO. 76:
IGKV4-9-1*01 (RC)
L1:
ATGATGTCACAGACACAGGTCCTCTTGTCGGTGTTTCTCTGGGTCTCAG
L2:
GTGCCTGTGGG
VK:
GACCTCGTGATGACGCAGTCTCCAGGCTCCTTGGCAGCGTCTCTA
GGACAGAGAGTCGAGATGAAGTGCAAGGCCAGTCAGAGTGTTAGC
AGCTACTTAGACTGGTACCAGCAGAAACCAGGACAGGCTCCTAAG
CAGCTCATCTATGCTGCATCCAGCAGAGCGTCTGGGGTCCCCGAC
CGATTCAGTGGCAGTGGATCTGGGACAGATTTCACTATCACCATC
AGCAGCCTCCAGGCTGAAGATCTGGCCATTTATTACTGTCAGCAG
TATAATAGTGCTCCTCC
SEQ ID NO. 77:
IGKV4-9*01 (RC)
L1:
ATGATGTCACAGACACAGGTCCTCTTGTCGGTGTTTCTCTGGGTCTCAG
L2:
GTGCCTGTGGG
VK:
GACCTCGTGATGACGCAGTCTCCAGGCTCCTTGGCAGCGTCTCTA
GGACAGAGAGTCGAGATGAAGTGCAAGGCCAGTCAGAGTGTTAGC
AGCTACTTAGACTGGTACCAGCAGAAACCAGGACAGGCTCCTAAG
CAGCTCATCTATGCTGCATCCAGCAGAGCGTCTGGGGTCCCCGAC
CGATTCAGTGGCAGTGGATCTGGGACAGATTTCACTATCACCATC
AGCAGCCTCCAGGCTGAAGATCTGGCCATTTATTACTGTCAGCAG
TATAATAGTGCTCCTCC
SEQ ID NO. 78:
IGKV4-8*01 (RC)
L1:
ATGATGTTGCAGACACAGGTCCTTATAACCTTGTTGCTCTGGGTCTCAG
L2:
GTGCCTGTGGG
VK:
GACATCGTGATGACCCAGTCTCCAGACTCCTTGTCTGTGTCTGCA
GGACAAAGGGTCGACATGAAGTGCAGGGCCAGTCAGAGTGTTAGC
AATGAGTTATCCTGGTACCAGCAAAAACCAGGACAGGCTCCTAAG
CTGCTGATCTATGCAGCATCCAACAGAGCATCTGTGGTCCCTGAC
CGATTCAGTGGCGGTGGATCTGGGACAGATTTCACTCTCACCATC
AGTAGCCTCCAGGCTGAAGATGTGGCCGTTTATTACTGTCTGCAG
CATTATAATAATCCTCC
SEQ ID NO. 79:
IGKV4-5-1*01 (RC)
L1:
ATGATGTCGCTGACAAAGGTCCTTATATCTGTGTTGCTCTGGGTCTCAG
L2:
GTGCCTGTGGG
VK:
GACATCGTGTTGACCCAGTCTCCAGAGTCCTTGGCAGTGTCTCTA
GGACAGAGGGTCGAGATGAAGTGCAAGGCCAGTCAGAGTGCTAGC
AGCAACTTGGACTGGCACCAGCACAAACCAGGACAGGCTCCTAAG
CAGCTCATCTACAGAGCATCCAGCAGAGCGTCTGGGGTCCCTGAC
CGATTCAGTGGCAGTGGATCTGGGACAGATTTCACTCTCACCATC
AGCAGCCTCCAGGCTGAAGATGTGGCCGTTTATTACTGTCAGCAG
TATAATAGTGCTCCTCC
SEQ ID NO. 80:
IGKV5-5*01 (RC)
L1:
ATGATGTCATGGACTCAGATCCTTATGTCCTTGTTGCTCTGGGTCTCAG
L2:
GTGCCTGTGGG
VK:
GACATCGTGATGACCCAGTCTCCAGACTCCTTGGCAGTGTCTCTA
GGACAGAGAGTCGAGATGAAGTGCAAGGCCAGTCAGAGTGTTAGC
AACTACTTAGACTGGTACCAGCAAAAACCAGTAAAGGCTCCTAAG
CTGCTCATCTATGCAGCATCCAGCAGAGCATCTGGGGTCCCCGAC
CGATTCAGTGGCAGTGGATCTGGGACAGATTTCACTCTCACCATC
AGCAGCCTCCAGGCTGAAGATGTGGCAGTTTACTCCTGTCAGCAG
CGTTATAGTTCTCCTCC
SEQ ID NO. 81:
IGKV4-2*01 (RC)
L1:
ATGATGTCGCTGACACAGTTCCTTATATCTGTGTTGCTCTGGGTCTCAG
L2:
GTGCCTGTGGG
VK:
GACATCGTGATGACGCAGTCTCCAGACTCCTTGGCAGTGTCTCTA
GGACAGAGAGTCGAGATGAAGTGCAAGGCCAGTCAGAGTGTTAGC
AGCAGCTTGGACTGGCACCAGCACAAACCAGGACAGGCTCCTAAG
CTGCTCATCTACAGAGCATCCAGCAGAGCGTCTGGGGTCCCTGAC
CGATTCAGTGGCAGTGGATCTGGGACAGATTTCACTCTCACCATC
AGCAGCCTCCAGGTTGAAGATGTGGCAGTTTATTACTGTATGCAG
TCTAATACTGCTCCTCC
SEQ ID NO. 82:
IGKV4-1*01 (RC)
L1:
ATGCTGACGCAGACGCAGGTCCTTATATCTGTGTTGCTCTGGGTCTCAG
L2:
GAGCCTGTGGG
VK:
GACGTCATGATGACCCAGTCTCCAGACTCCTTGGCAGCGTCTCTA
GGACAGAGAGTCGAGATGAAGTGCAAGGCCAGTCAGAGTGTTAGC
AGCTACTTAGCTTGGTACCAGCACAAACCAGGACAGGCTCCTAAG
CGGCTCATCTATGCTGCATCCAGCAGAGCATCTGGGGTCCCTGAC
CGATTCAGTGGCAGTGGATCTGGGATGGATTTCACTCTCACCATC
AGCAGCCTCCAGGCTGAAGATGTGGCCATTTATTACTGTATGCAG
CATTATAATAATCCTCC
IGKJ
SEQ ID NO. 83:
IGKJ1*01
GTGGACGTTCGGTGCCGGGACCAAGCTGGAAATCAAAC
SEQ ID NO. 84:
IGKJ2*01
TATACACGTTTGGCCAAGGGACCAAGCTGGAGATCAAAA
SEQ ID NO. 85:
IGKJ3*01
GTTCACTTTCGGCCAAGGGACCAAACTGGAGATCAAAC
SEQ ID NO. 86:
IGKJ4*01
GCTTACGTTCGGCCAGGGGACCAAGCTGGAGATCAAAC
NB. The sequence of the horse lambda locus
is still incomplete. The sequences below do
not necessarily describe the complete
equine IGLV and IGLJ repertoire.
IGLV
SEQ ID NO. 87:
IGLV28 (RC)
L1:
ATGGCCTGGTCCCCGCTCCTCCTCACCCTCATCGCTCTCTGCACAG
VL:
TATCCTGGGCCCAGTCTGTGCTGACTCAGCCGGCCTCAGTGTCTG
GGAACCTGGGCCAGAGGGTCACCATCTCCTGCACTGGGAGCAGCT
CCAACACCAGGGATAATTATGTGAACTGGTACCAGCAGCTCCCAG
GAACCGCCCCCAAACTCATCATCTATGAAAATAGCAAAAGACCCT
CCGGGACCCCAGATCGAATCTCTGGCTCCAAGTCTGGAAACCCGG
CCTCCCTGACCATCACTGGGCTCCAGGCTGAGGATGAGGCTGATT
ATTACTGCCAGTCCTATGATGACAACCTGAATGCTCG
SEQ ID NO. 88:
IGLV27
L1:
ATGGCCTGGTCCCCTCTCCTCCTCACCCTCATCGCTCTCTGCACAG
VL:
TCACTACTATCACTGCTGTAATAGGAACCACAGTAATAATCGGCC
TCGTCCTCAGCCTGAAGCCCAGAGATGGTCAGGGTGGCTGTGTTG
CCAGACTTGGAGCCAGAGAATCGATCTGGGACCCCTGAGGCTCGT
TTGTTATTACCATAGATGAGGGTTTTGGGGGCTGTTCCTGGGATC
TGTTGGTACCAGCCCACAGCACTATAACTATACCCGATGTTGGAG
CTGCTTCCAGAGCAGGAGATGGTGACTGTCTGGCCCAGGGTCCCA
GACACTGAGGCGGGCTGGGTCAGAGACTGGGCCCAGGATC
SEQ ID NO. 89:
IGLV26
L1:
ATGGCCTGGTCCCCTCTCCTCCTCACCCTCATCGCTCTCTGCACAG
VL:
GATCCTGGGCCCAGTCTCTGACTCAGCCCGCCTCAGTGTCTGGGA
CCCTGGGCCAGACAGTCACCATCTCCTGCTCTGGAAGCAGCTCCA
ACATCGGGTATAGTTATAGTGCTGTGGGCTGGTACCAACAGATCC
CAGGAACAGCCCCCAAAACCCTCATCTATGGTAATAACAAACGAG
CCTCAGGGGTCCCAGATCGATTCTCTGGCTCCAAGTCTGGCAACA
CAGCCACCCTGACCATCTCTGGGCTTCAGGCTGAGGACGAGGCCG
ATTATTACTGTGGTTCCTATTACAGCAGTGATAGTAGTGA
SEQ ID NO. 90:
IGLV25
L1:
ATGGCCTGGTGCCCTCTCCTCCTCACCCTCATCGCTCTCTGCACAG
VL:
GATCCTGGGCCCAGTCTGTGACTCAGCCCGCCTCAGTGTCTGGGA
CCCTGGGCCAGACAGTCACCATCACCTGCACTGGAAGCAGCTCCA
ACATAGTTGCTTATGTGGGCTGGTACCAACAGATCCCAGGAACAG
CCCCCAAAACCCTCATCTACGCTAATAACAAACGAGCCTCAGGGG
TCCCAGATCGATTCTCTGGCTCCAAGTCTGGCAGCACAGCCACCC
TGACCATCACTGGGCTCCAGGCTGAGGACGAGGCCGATTATTACT
GTGGTACCTCTAGCAGCAGTGGTAGTAGTGA
SEQ ID NO. 91:
IGLV24
L1:
ATGTCCTGTACTCCTCTCCTCCTCGTGCTCCTCTCTCACTGCACAG
VL:
GTTCCCTCTCCCAGCCTGTGCTGACCCAGCCTCCCTTCTTCTCTG
CATCTCCTGGAGCATCAGCCAGACTCACCTGCACCCTGAGCAGTG
ACATCAGTGTTGACAGCTCTCTCATATTCTGGTGCCAGCAGAAGC
CAGGGAGCCCTCCCCGGTATCTCCTGAGTTTCTACTCAGACTCAG
TTAAGCACCAGGGCTCCGGGGTCCCCAGCCGCTTCTCTGGATCCA
GAGACACCTCGGCCAATGCAGGGCTTCTGCTCATCTCTGGGCTCA
AGGCTGAGGACGAGGCTGACTATTACTGTGCTACAGCTGATAGCA
GTGGGAGCAGCTCTGGTTACT
SEQ ID NO. 92:
IGLV23
L1:
ATGGCCTGGTCCCCTCTCCTCCTCACCCTCATCGCTCTCTGCACAG
VL:
GATCCCGGGCCCAGTCTGTGACGCAGCCCGCCTCAGTGTCTGGGA
CCCTGGGCCAGACAGTCACCATCTCCTGCTCTGGAAGCAGCTCCA
ACATCGGGAGTGGTCATGTGTCCTGGTACCAACAGATCCCAGGAA
CAGCCCCCAAACGCCTCATCTATTCTTCCGCTAGCAGGGCTTCCA
GGGTCCCCGACCGATTCTCTGGCTCCAGGTCTGGCAACACAGCCA
CCCTGACCATCTCTGGGCTCCAGGGTGAGGACGAGGCCGATTATT
ACTGTGGTACATTGTACAGCAGTTGGAGTAGTGA
SEQ ID NO. 93:
IGLV22
L1:
ATGGCCTGGTCCCCTCTCCTCCTCACCCTCATCGCTCTCTGCACAG
VL:
GATCCTGGGCCCAGTCTCTGACTCAGCCCGCCTCAGTGTCTGGGA
CCCTGGGCCAGACAGTCACCATCTCCTGCTCTGGAAGCAGCTCCA
GCATAGGTTCTTATATGGGCTGGTACCAACAGATCCCAGGGACAG
CCCCCAAAACCCTCATCTATGCTACTAACAAACGAGCCTCAGGGG
TCCCAGATCGATTCTCTGGCTCCAAGTCTGGCAACACAGCCACCC
TGACCATCTCTGGGCTTCAGGCTGAGGACGAGGCCGATTATTACT
GTGGTTCCTATTACAGCAGTGATAGTAGTGA
SEQ ID NO. 94:
IGLV21
L1:
ATGGCCTGGACAGTGCTTCTTCTCTGGCTCCTCACTTACAGCTCAG
VL:
GGGCAGATTCTCAGGCTGTGGTGATCCAGGACCCATCATTCTCTG
TGTCCCTAGGGGGGACGGTCATACTGACCTGTGGCCTTAGAACTG
GGTCAGTCTCTACCAGTAACTATCCTAGATGGTACCAGCAGACAC
CAGGCAAGGCTCCCCGTACACTCACCTACAGCACAAACAACCGCC
CCTCTGGGATCCCTGAACGCTTCTCTGGATCCATCTCAGGAAACA
AAGCCGCCCTCACCATCACGGGGGCCCAGCCCGAGGACGAGGCCG
ACTATTACTGTGATCTGTATGTGGATCGTGGTGTTTC
SEQ ID NO. 95:
IGLV20 (RC)
L1:
ATGGCCTGGATGGTGCTTCTTCTCGGGCTCCTTTCTTACAGCTCAG
VL:
GGGCGGATTCTCAGTCTGTGGTGACCCAGGAGCCATCACTCTCAG
TGTCTTCAGGAGGGACAGTCACACTCACCTGTGGCCTTAACTCTG
GGTCAGTCTCTTCCAGTAACCACCCCAGCTGGCACCAGCAAACCC
CAGGCCAGGCTCCCCGCACACTTATCTACTACACAAACACCCGTG
CCTCTGGAGTCCCTAATCTCTTCTCTGGATCCATCTCCGGGAACA
GAGCCACCCTCACCATCACGGGGGCCCAGCGTGAGGACGAGGCCG
ACTATTACTGCGCTCTGTATACGGGTAGTTACACTGA
SEQ ID NO. 96:
IGLV19 (RC)
L1:
ATGGCCTGGTCCCCTCTCCTCCTCACCCTCATCGCTCTCTGCACAG
VL:
GATCCTGGGCCCAGTCTGTGACTCAGCCCGCCTCAGTGTCTGGGA
CCCTGGGCCAGACAGTCACCATCTCCTGCACTGGAAGCATCTCCA
ACATAGGTGTTTATGTGGACTGGTACCAACAGATCCCAGGAACAG
CCCCCAAAACCATCATCTATGCTACTAACAAACAACCCTCAGGGG
TCCCAGATCGATTCTCTGGCTCCAAGTCTGGCAACACAGCCACCC
TGACCATCACTGGGCTCCAGGCTGAGGACGAGGCTGATTATTACT
GTGGTATCTATGACAGCAGCCTGAGTAGTGA
IGLJ
NB IGVL gene segments exist both upstream
and downstream of the equine IGLJ gene
segments and IGLC genes (not shown).
SEQ ID NO. 97:
IGLJ7
J7:
TGCGATGGTCAGGTGGGTGCCTCCGCCGAATGCACCA
SEQ ID NO. 98:
IGLJ6
J6:
TGCGATGGTCAGGTGGGTGCCTCCGCCGAATGCACCA
SEQ ID NO. 99:
IGLJ5
J5:
TGCGATGGTCAGGTGGGTGCCTCCGCCGAATGCACCA
SEQ ID NO. 100:
IGLJ4
J4:
GCGATGGGTCAGGTGGGTGCCTCCGCCGAATACAGCACA
SEQ ID NO. 101:
IGLJ3
J3:
AGGACTATCAGCTGGGTCCCTCAGCTGAGCACAGGA
SEQ ID NO. 102:
IGLJ2
J2:
CTAGGACGGTCAGATGGGTACCTCCAGTGAACTATGAA
SEQ ID NO. 103:
IGLJ1
J1:
CTAGGACGGTCAGATGGGTACCTCCAGTGAACTATGAA
SEQ ID NO. 104:
IGLV2 (RC)
L1:
ATGGCCTGGACCCCTCTCCTGCTCCTCCTCCTCACTCTC
TGCACAG (RC)
VL:
GCTCTGTGGCTTCTTCTATGCTGACTCAGCCACTTACCTTGTCCG
TGGCCTTTGGAAGCACAGTCACTATCACATGCCAGGGAGAGCTCC
TAGACAGTTATTATGCTGAGTGGTACCAGCAGAAGCCAGACCAGG
CTCCCGTGCTGGTCATATATTATGGAAGCAAACGTCCTTCGGGGA
TTTCTACCCGATTCTCTGGCTCCTACTCAAGCAAGATGGCCACCC
TGACCCTCAGTGGGGCCTTGGCCGAGGATGAGGCTGACTATTACT
GTCAGGTGTGGGACAGCAGTGGTAACCAGCC
SEQ ID NO. 105:
IGLV3 (RC)
L1:
ATGGCCTGGACCCTTCTCCTGCTTCCCCTCCTCACTCTCTGCACAG
VL:
GTTCTGTGACCACCTATGACTTGACGCAACCACACTCAACTTCGG
TGGCCCTAGGACAGACAGCGACAATCACCTGCTCTGGAGATAATC
TCGAGGATGAATATGCTTACTGGTACCAGCAGAAGACAGGCCAGT
CCCCTGCCCTGGTCATTTATAAGGATAGTGAGCACCCCTCAGGGA
TCCCTGACCGGTTCTCTGGCTCAAACTCAGGAAACACAGCCACGC
TGACCATCAGAGGGGCCAAGACAGAGGACAAGGCTGACTATTACT
GCCAATCGTGGAGCAGTGCTAATGCT
SEQ ID NO. 106:
IGLV4 (RC)
L1:
ATGGCCTGGACCCCTCTCTTGTTTGCCTTCCTCACTCTCTGCACAG
VL:
GTCCTGTAGTCTCTTCTGAGGTGACTCAGCCAACTGCGGTGTCCG
TGGCCTTGGGACAGACAGCCTCCATCACCTGCCAGGGAAGCGACT
TTGAAAATTATTATGCTAGCTGGTACCAGCAGAAGCCAGGCCAGG
CCCCAGTGCTGGTCATCAATGCTAATAATGAGCGGCCCTCAGGGA
TCCCTGAACGATTCTCTGGGTCCAGTTCAGGAGAGACAGCTACGC
TGACCATCAGTGGAGCCCACGCTGAGGACGAGGCCGACTATTACT
GTCTGGCAACAGATGCTTATGTTGCTGAAGCT
SEQ ID NO. 107:
IGLV5 (RC)
L1:
CTGTGCAGAGAGTGAGGAAGGCTAACAAGAGAGGGGTCCAGGCCAT
VL:
AGCTTCATAATCAGAAGCATCTGCTGCCAGACAGTAATAGTCAGC
CTCGTCCTCAGCCTGGGCCCCGCTGATGGTCAGCGTGGCTGTGTC
TCCTGAGCTGGAGCCAGAGAATCGTTCAGGGATCCCTGAGGGCCG
CTCATTACTAGCATCGATGACCAGCACAGGGGCCTGGCCTGGCTT
CTGCTGGTACCAGCTACCAACAAAACTTTCAAAGTCGCCTCCCTT
GCAGGTGATGGTGGCCGTCTGTCCCAAGGCCACAGACACTGAAGA
TGGCTGAGTCAGCTTAGAAGAGGCCACGGGAC
SEQ ID NO. 108:
IGLV6 (RC)
L1:
ATGGCCTGGACCCCTCTCTTGTTAGCCTTCCTCACTCTCTGCACAG
VL:
GTCCTATGGCCTCTTCGGAGGTGACTCAGCCATCTGCGGTGTCTG
TGGCCTTGGGACAGACAGCCACCCTCACCTGCCAGGGAGACTACT
ATGAAAGATATATTGTCAACTGGTACCAGCAGAAGCCAGGCCAGG
CACCTGTGCTGGTCATCTATGCTAATAGTGAGCGGCCCTCAGGAA
TCCCTGAACGATTCTCTGGCTCCAGCTCATTAGGCACATCCACGC
TGACCATCAGCGGGGCCCAGGCTGAGGATGAGGCTGACTATTACT
GTCAGCCAGCAGATGCTCATCGTTCTGAATCTGTCCTATGGCCTC
TTCGGAGGTGACTCAGCCATCTGCGGTGTCTGTGGCCTTGGGACA
GACAGCCACCCTCACCTGCCAGGGAGACTACTATGAAAGATATAT
TGTCAACTGGTACCAGCAGAAGCCAGGCCAGGCACCTGTGCTGGT
CATCTATGCTAATAGTGAGCGGCCCTCAGGAATCCCTGAACGATT
CTCTGGCTCCAGCTCATTAGGCACATCCACGCTGACCATCAGCGG
GGCCCAGGCTGAGGATGAGGCTGACTATTACTGTCAGCCAGCAGA
TGCTCATCGTTCTGAATCT
SEQ ID NO. 109:
IGLV7 (RC)
L1:
ATGGCCTGGACCCCTCTCTTGTTAGCCTTCCTCTCTCTCTGCACAG
VL:
GTCCTGTTGTCTCTTCTGCAGTGACTCAGCCATCTGAGGTGTCCG
TGGCCTTGGGACAGAGAGCCACCCTCACCTGCCAGGGAAGCAACT
TTGAATTTTTTTCTCCTAGCTGGTACCAGCAGAAGCCAGGCCAGG
CCCCTGTACTGCTCATCAATATTAATAATGAGCGCCACTCAGGGA
TCCCTGAACGATTCTCCGGCTCCAGCTCAGGAGACACGTCCACAC
TGACCATCAGTGGGGCCCAGGCTGAGGACGAGGCTGACTATTACT
GTCTGGCAGTAGATGCTCTTAGTTCTGAAACT
SEQ ID NO. 110:
IGLV8 (RC)
L1:
ATGGCCTGGACACTTCTCCTTCTCCCTCTCCTCACTCTCTGCACAG
VL:
GTTCTGTGGCCCCTTCTGAGCTGACTCAGTTAACTGTGGTGTCTG
TGGCCTTGGCACAGACAGCCAGGGTCACCTGCCAGGGAGAGAGAC
CAAAAAGTGTCTATGCTGGCTGGTACCAGCAGAAGCCAGGCCAGG
CCCCTGTATGGGTCATCTATAGTAAAAACAATTGGACCACAGGCA
CACCTGAACAATTCTCTGCCTCTGACTCAGGGGACACAGCCACCC
TGACCATCAGTGGGGTCCAGGTTGAGGGCGAGACTGACTATTACT
GTGGGGTAAGTGTTGGAAGTGGGAGCAGCTGGCAGTCACT
SEQ ID NO. 111:
IGLV9 (RC)
L1:
ATGGCCTGGACCCCTCTCCTGCTCCCTCTCCTCACTCTCTGCACAG
VL:
GTTCTGTGTCCTCTTCTGAGCTTACTCAGTCTACTGCAGTGTCAT
TTTCCTTGGGACAGACAGCCACCATCACCTGCCAGGGAGAAACCC
TAAGAAGCCACTATGCTAGCIGGTACCAGAAGAATCCAGGACAGG
CCCCTGTATTGGTAATATATGGTAATAACAACCGGCCCTCAGGGA
TCCCTGCCCGATTTTCCAGCTCCTACTCAGAGGACACAGGCACCC
TGACCATCAGTGGGGTCCAGATAGAGGATGAGGCTGACTATTACT
GCCAATCATTGGGCAGTGATTATGC
T
SEQ ID NO. 112:
IGLV10 (RC)
L1:
ATGGCCTGGGCTCTGTTCCTCATCACCCTCCTCACTCAGGGCACAG
VL:
GGTCCTGGGGCCAGTCTGCCCTGGTTCAGCCTTCTTCGGTGTCCG
TGGCTCTAGGACAGTCGGTCACCATCTCCTGTGCTGGAAGCAGCA
GTGACATTGGGTATTATAACTCTATTTCCTGGTACCAACAGCACC
CAGGCACAACCCCAAAGCTGCTGATTTACTATACCAATAAGAAGC
ACTCAGGGATCCCTGATCGCTTCTCTGGCTCCAAGTCTGGGAACA
CGGCCTCCCTGACCATCTCTGGGCTCCAGGCTGAGGATGAGGCTG
AGTATTACTGTTGCTCATATGCAGGCAGTGGCAATTTA
SEQ ID NO. 113:
IGLV11 (RC)
L1:
ATGGCCTGGACTCTGCTCCTTCTCACCCTCCTCACTCAGGGTACAG
VL:
GGTCCTGGGCCCAGTCTGCCCTGACTCAGCCTGCGTCAGTGTCCG
GGGCTCTAGGACAGTCGGTCACCATCACCTGTGCTGGAAGCAGCA
GTGACATTGGGGGTTATAATGCTGTCAGCTGGTTACAACAGCACC
CGGGCACAGCCCCCAAAGTTCTGATTTATAGTGTGAATACTCGGG
CCTCAGGGATCCCTGATCGCTTCTCTGGCTCCAAGTCTGGCAACA
CGGCCTCCCTGACCATCTCTGGGCTCCAGGTTGAGGACGAGGCTG
ATTATTACTGTTACTCGCTTGTGAGTGGTTACACTTTC
SEQ ID NO. 114:
IGLV12 (RC)
L1:
ATGGCCTGGGCTCTGCTCCTCATCAGCCTCTTCACTCAGGGCACAG
VL:
GGTCCTGGGCGCAGTCTGCCCTGACTCAGCCTGCGTCAGTGTCCG
GGACTCTGGGACAGTCGGTCACCATCTCCTGTGCTGGAAGCAGCA
GCAACATTGGGAGTTATAACTATGTTTCCTGGTACCAACAGCACC
CGGGCACAGCCCCCAAACTCCTCATTTATAGTGCCAGTTCTCGAG
CCTCAGGGATCCCTGATCGCTTCTCTGGCTCCAAGTCTGGGAACA
CGGCCTCTCTGACCATCTCGGGGCTCCAGGCTGAGGACGAGGCCG
ATTATTACTGTAGCTCATATATCAATGCTGATCCTTATC
SEQ ID NO. 115:
IGLV13 (RC)
L1:
ATGGCCTGGGCTCTGCTCCTCATCACCCTCCTCACTCAGGGCACAG
VL:
GTAATTGTAACTGCCAACATACGCGTGACAGTAATAATCAGCCTC
GTCCTCAGCCTGGAGCCCAGAGATGGTCAGGGACATCGTGTTGCC
AGACGTGGAGCCGGAGAAGCGATCAGGGATCCCTGAGGCCCGATT
ATTCCCATTATAAATGAGGAGTTTGGGGGCTGTGCCTGGGTGCTG
TTGGTACCAGGAAATATATTTATAAGATCCCGTGCTTCCAGCACA
GGTGATGGTGACCGACTGTCCCAGAGTCCCGGAGACTGACGCAGG
CTGAGTCAGGGCAGACTGCGCCCAGGACC
SEQ ID NO. 116:
IGLV14 (RC)
L1:
ATGGCCTGGGTGCCACTCCTGCTCACACTTCTGGCTCACTGCACAG
VL:
GGTCCACTTCACAGGATGTGGTGATTCAGGAATCTTCACTGATCA
CAACTCCTGGGGGAACAGTCACACTCACCTGTGGCTCAAGTGCTG
GGGCTGTCACCTCCAATAATTATGCCAACTGGGTCCAAGAGAAGC
CCTATCAGGGACGCCAGGGTCTAATAGGTGGTACTAGCAACAGGG
TCTCAGGGGGTCCCTGCCCGATTCTCTGGCTCCCTGCGCTTGGGA
ACAAGGCCGCCCTCACTATCATGGGGGCCCAGCCAGAGGACGAGG
ACGAGTGTTACTGTGCTCTGTGGTTCAGCAACCATTTC
SEQ ID NO. 117:
IGLV15 (RC)
L1:
ATGGCCTGGTCCCCTCTCCTCCTCACCCTCATCGCTCTCTGCACAG
VL:
GATCCTGGGCCCAGTCTCTGACTCAGCCCGCCTCAGTGTCTGGGA
CCCTGGGCCAGACAGTCACCATCTCCTGCTCTGGAAGCAGCTCCA
ACATCGGGTATAGTTATAGTGCTGTGGGCTGGTACCAACAGATCC
CAGGAACAGCCCCCAAAACCCTCATCTATGGTAATAACAAACGAG
CCTCAGGGGTCCCAGATCGATTCTCTGGCTCCAAGTCTGGCAACA
CAGCCACCCTGACCATCTCTGGGGTCCAGGCTGAGGACGAGGCCG
ATTATTACTGCTCAGCAGGAGACAGCAGTGGTAGTAGTGA
SEQ ID NO. 118:
IGLV16 (RC)
L1:
ATGGCCTGGACTCCTCTCATCCTCATGCTCCTGTCTCACTGCACAG
VL:
GTTCCCTCTCCCAGCCTGTGCTGACCCAGCCACCCTCCCTCTCTG
CATCTCCTGGAACATCAGCCAGACTCACCTGCGCCCTGAGCAGTG
ATGTCAGTGTTAGCAGCTCTCTCATATTCTGGTACCAGCAGAAGC
CAGGGAGCCCTCCGGGGTATCTTCTGAGTTTCTACTCAGACTCAG
TTAAGCACCAGGGCTCCGGGGTCCCCAGCCACTTCTCTGGATCCA
AAGACACCTCGTCCAATGCAGGGCTTCTGCTCATCTCTGGGCTCG
AGGCTGAGGACGAGGCTGACTATTACTGTGCTACAGCTGATAGCA
GTGGGATCAGCTCTGGTTACT
SEQ ID NO. 119:
IGLV17 (RC)
L1:
ATGGCCTGGTCCCCTCTCCTCCTCACCCTCATCGCTCTCTGCACAG
VL:
GATCCTGGGCCCAGTCTGTGACTCAGCCCGCCTCAGTGTCTGGGA
CCCTGGGCCAGACAGTCACCATCTCCTGCTCTGGAAGCAGCTCCA
ACATCGGGTATAGTTATAGTTATGTGGGCTGGTTCCAACAGATCC
CAGGAACAGCCCCCAAAACCCTCATCTATGGTAATAACAAACGAG
CCTCAGGGGTCCCAGATCGATTCTCTGGCTCCAAGTCTGGCAACA
CAGCCACCCTGACCATCTCTGGGGTCCAGGCTGAGGACGAGGCCG
ATTATTACTGTGGTTCCTATGACAGCAGCAGTAGTAGTGA
SEQ ID NO. 120:
IGLV18 (RC)
L1:
ATGGCCTGGTCCCCTCTCCTCCTCACCCTCATCGCTCTCTGCACAG
L2:
TCCCGGGCCCAGTCTGTGACTCAGCCCGCCTCAGTGTCTGGGACC
CTGGGCCAGACAGTCACCATCTCCTGCTCTGGAAGCAGCTCCAAC
ATCGGGAGTGGTCATGTGTCCTGGTACCAACAGATCCCAGGAACA
GCCCCCAAACGCCTCATCTATTCTTCCACTAATAGGGCTTCTGGG
GTCCCCGACCGATTCTCTGGCTCCAGGTCTGGCAACACAGCCACC
CTGACCATCTCTGGGCTCCAGGCTGAGGACGAGGCTGATTATTAC
TGTGGTACATTGTACAGCAGTTGGAGTAATGA
SEQ ID NO. 121:
IGLV19 (RC)
L1:
ATGGCCTGGTCCCCTCTCCTCCTCACCCTCATCGCTCTCTGCACAG
LV:
CAGTCTGTGACTCAGCCCGCCTCAGTGTCTGGGACCCTGGGCCAG
ACAGTCACCATCTCCTGCACTGGAAGCATCTCCAACATAGGTGTT
TATGTGGACTGGTACCAACAGATCCCAGGAACAGCCCCCAAAACC
ATCATCTATGCTACTAACAAACAACCCTCAGGGGTCCCAGATCGA
TTCTCTGGCTCCAAGTCTGGCAACACAGCCACCCTGACCATCACT
GGGCTCCAGGCTGAGGACGAGGCTGATTATTACTGTGGTATCTAT
GACAGCAGCCTGAGTAGTGA
SEQ ID NO. 122:
IGLV20 (RC)
L1:
ATGGCCTGGATGGTGCTTCTTCTCGGGCTCCTTTCTTACAGCTCAG
VL:
GGGCGGATTCTCAGTCTGTGGTGACCCAGGAGCCATCACTCTCAG
TGTCTTCAGGAGGGACAGTCACACTCACCTGTGGCCTTAACTCTG
GGTCAGTCTCTTCCAGTAACCACCCCAGCTGGCACCAGCAAACCC
CAGGCCAGGCTCCCCGCACACTTATCTACTACACAAACACCCGTG
CCTCTGGAGTCCCTAATCTCTTCTCTGGATCCATCTCCGGGAACA
GAGCCACCCTCACCATCACGGGGGCCCAGCGTGAGGACGAGGCCG
ACTATTACTGCGCTCTGTATACGGGTAGTTACACTGA
Pre-DJ
This is a 21609 bp fragment upstream of the
Ighd-5 DH gene. The pre-DJ sequence can
be found in Mus musculus strain C57BL/6J
chromosome 12, Assembly: GRCm38.p4,
Annotation release 106, Sequence ID:
NC_000078.6
The entire sequence lies between the two 100 bp
sequences shown below:
Upstream of the Ighd-5 DH
gene segment, corresponding to positions
113526905-113527004 in NC 000078.6:
ATTTCTGTACCTGATCTATGTCAATATCTGTACCATGGCTCTAGC
AGAGATGAAATATGAGACAGTCTGATGTCATGTGGCCATGCCTGG
TCCAGACTTG
(SEQ ID NO. 123)
2 kb upstream of the Adam6a gene corresponding
to positions 113548415-113548514
in NC 000078.6:
GTCAATCAGCAGAAATCCATCATACATGAGACAAAGTTATAATCA
AGAAATGTTGCCCATAGGAAACAGAGGATATCTCTAGCACTCAGA
GACTGAGCAC
(SEQ ID NO. 124)
Adam6a
Adam6a (a disintegrin and metallopeptidase
domain 6A) is a gene involved in male
fertility. The Adam6a sequence can be found
in Mus musculus strain C57BL/6J chromosome 12,
Assembly:
GRCm38.p4, Annotation release 106,
Sequence ID:
NC_000078.6 at position 113543908-113546414.
Adam6a sequence ID:
OTTMUSG00000051592 (VEGA)

We claim:

1. A transgenic rodent with a genome in which an endogenous a rodent immunoglobulin variable gene locus is deleted and replaced with a partly equine immunoglobulin locus comprising equine immunoglobulin variable gene coding sequences and non-coding regulatory sequences based on the endogenous rodent immunoglobulin variable gene locus, wherein the partly equine immunoglobulin locus of the transgenic rodent is functional and expresses immunoglobulin chains comprising equine variable domains and rodent constant domains.

2. The transgenic rodent of claim 1, wherein the partly equine immunoglobulin locus comprises equine V_{H}, D_{H} and J_{H} coding sequences.

3. The transgenic rodent of claim 1, wherein the partly equine immunoglobulin locus comprises equine kappa V_{L} and J_{L} coding sequences, equine lambda V_{L} and J_{L} coding sequences, or a combination thereof.

4. The transgenic rodent of claim 1, wherein the partly equine immunoglobulin loci comprise equine V_{H}, D_{H} and J_{H}, equine kappa V_{L} and J_{L} coding sequences, equine lambda V_{L} and J_{L} coding sequences, or a combination thereof.

5. The transgenic rodent of claim 1, wherein the rodent is a mouse.

6. The transgenic rodent of claim 1, wherein the non-coding regulatory sequences comprise promoters preceding individual V gene segments, splice sites, and recombination signal sequences for V(D)J recombination.

7. The transgenic rodent of claim 1, wherein the partly equine immunoglobulin locus further comprises an ADAM6 gene.

8. The transgenic rodent of claim 1, wherein the partly equine immunoglobulin locus further comprises Pax-5-Activated Intergenic Repeat (PAIR) elements.

9. The transgenic rodent of claim 1, wherein the partly equine immunoglobulin locus further comprises CTCF binding sites from a heavy chain intergenic control region 1.

10. A cell of B lymphocyte lineage from the transgenic rodent of any of claims 1 to 9.

11. A hybridoma cell derived from the cell of B lymphocyte lineage of claim 10.

12. An immortalized cell derived from the cell of B lymphocyte lineage of claim 10.

13. A part of or whole immunoglobulin molecule comprising equine variable domains and rodent constant domains derived from the cell of B lymphocyte lineage of claim 10, the hybridoma of claim 11 or the immortalized cell of claim 12.

14. A method for generating the transgenic rodent of claim 1, the method comprising:
a) integrating in a rodent cell's genome at least one target site for a site-specific recombinase upstream of an endogenous immunoglobulin variable gene locus and at least one target site for a site-specific recombinase downstream of the endogenous immunoglobulin variable gene locus, wherein the endogenous immunoglobulin variable locus comprises (i) V_{H}, D_{H} and J_{H} gene segments, (ii) V_{K} and J_{K} gene segments, (iii) Vλ and JA gene segments; or (iv) Vλ and JA gene segments and CA genes;
b) providing a vector comprising a partly equine immunoglobulin locus, the partly equine immunoglobulin locus comprising partly equine immunoglobulin variable region gene segments, wherein each of the partly equine immunoglobulin variable region gene segments comprises equine immunoglobulin variable region gene coding sequences and rodent non-coding regulatory sequences, with the partly equine immunoglobulin variable region gene locus being flanked by target sites for a site-specific recombinase, wherein the target sites are capable of recombining with the target sites introduced into the rodent cell in step a);
c) introducing into the cell the vector of step b) and a site-specific recombinase capable of recognizing the target sites;
d) allowing a recombination event to occur between the genome of the cell and the partly equine immunoglobulin locus, resulting in a replacement of the endogenous immunoglobulin variable gene locus with the partly equine immunoglobulin locus;
e) selecting a cell that comprises the partly equine immunoglobulin variable locus generated in step d); and
f) utilizing the cell to create a transgenic rodent comprising the partly equine immunoglobulin variable locus.

15. The method of claim 14, wherein the cell is a rodent embryonic stem (ES) cell.

16. The method of claim 15, wherein the cell is a mouse embryonic stem (ES) cell.

17. The method of claim 14, further comprising after the introducing step and before the providing step a step of deleting the endogenous immunoglobulin variable gene locus by introduction of a recombinase that recognizes a first set of target sites, wherein the deleting step leaves in place at least two target sites in the rodent cell's genome that are not capable of recombining with one another.

18. The method of claim 14, wherein the vector comprises equine V_{H}, D_{H}, and J_{H}, coding sequences.

19. The method of claim 14, wherein the vector comprises V_{L} and J_{L} coding sequences, either κ or λ.

20. The method of claim 14, wherein the vector further comprises V gene promoters, splice sites, and recombination signal sequences of endogenous host origin.

21. The method of claim 14, wherein the vector further comprises an ADAM6 gene.

22. The method of claim 14, wherein the vector further comprises Pax-5-Activated Intergenic Repeat elements.

23. The method of claim 14, wherein the vector further comprises CTCF binding sites from a heavy chain intergenic control region 1.

24. A method of producing an antibody for therapeutic or diagnostic use, the method comprising:
(i) expressing an antibody with an equine variable domain cloned from an antibody-producing cell of a transgenic rodent whose genome comprises an endogenous rodent immunoglobulin locus variable region that has been deleted and replaced with an immunoglobulin locus variable region comprising at least one of each of a chimeric V_{H}, D and J_{H} immunoglobulin variable region gene segments at the immunoglobulin heavy chain locus, and/or at least one of each of a chimeric V_{L} and J_{L} variable gene segments at the immunoglobulin light chain loci, wherein each chimeric gene segment comprises equine V, D or J immunoglobulin variable region coding sequences and rodent immunoglobulin variable region non-coding gene segment sequences; and
(ii) isolating the antibody with the equine variable domain, wherein the antibody is suitable for therapeutic or diagnostic use.

25. The method of claim 24, wherein the antibody is cloned from a B cell of the transgenic rodent.

26. A therapeutic or diagnostic antibody produced by the method of claim 24.

27. A method of producing a therapeutic or diagnostic antibody with equine variable domains, the method comprising:
(i) cloning an equine variable domain of an antibody expressed by an antibody-producing cell from a transgenic rodent whose genome comprises an endogenous rodent immunoglobulin locus variable region that has been deleted and replaced with an immunoglobulin locus variable region comprising at least one of each of a chimeric V_{H}, D_{H} and J_{H} immunoglobulin variable region gene segments at the immunoglobulin heavy chain locus, and/or at least one of each of a chimeric V_{L} and J_{L} variable gene segments at the immunoglobulin light chain loci, wherein each chimeric gene segment comprises equine V, D or J immunoglobulin variable region coding sequences and rodent immunoglobulin variable region non-coding gene segment sequences; and
(ii) producing the therapeutic or diagnostic antibody comprising the equine variable domain of the antibody expressed by the transgenic rodent.

28. The method of claim 27, wherein the equine variable domain is cloned from an antibody expressed by a B cell from the transgenic rodent.

29. A therapeutic or diagnostic antibody produced by the method of claim 27.

30. A method of producing a monoclonal antibody comprising an equine variable domain, the method comprising:
(i) providing B cells from a transgenic rodent whose genome comprises an endogenous rodent immunoglobulin locus variable region that has been deleted and replaced with an immunoglobulin locus variable region comprising at least one of each of a chimeric V_{H}, D_{H} and J_{H} immunoglobulin variable region gene segments at the immunoglobulin heavy chain locus, and/or at least one of each of a chimeric V_{L} and J_{L} variable gene segments at the immunoglobulin light chain loci, wherein each chimeric gene segment comprises equine V, D or J immunoglobulin variable region coding sequences embedded in rodent immunoglobulin variable region non-coding gene segment sequences;
(ii) immortalizing the B cells; and
(iii) isolating monoclonal antibodies comprising equine variable domains expressed by the immortalized B cells, or genes encoding the antibodies.

31. The method of claim 30, further comprising:
(iv) cloning the equine variable domains expressed by the B cells; and
(v) producing a therapeutic or diagnostic antibody comprising the equine variable domain cloned from the B cells of the transgenic rodent.

32. A method of producing antibodies comprising equine variable domains, the method comprising providing a transgenic rodent whose genome comprises an endogenous rodent immunoglobulin locus variable region that has been deleted and replaced with an immunoglobulin locus variable region comprising at least one of each of a chimeric V_{H}, D_{H} and J_{H} immunoglobulin variable region gene segments at the immunoglobulin heavy chain locus, and/or at least one of each of a chimeric V_{L} and J_{L} variable gene segments at the immunoglobulin light chain loci, wherein each chimeric gene segment comprises equine V, D or J immunoglobulin variable region coding sequences embedded in rodent immunoglobulin variable region non-coding gene segment sequences, wherein the immunoglobulin locus of the transgenic rodent expresses antibodies comprising equine variable domains.

33. The method of claim 32, further comprising isolating the antibodies comprising equine variable regions expressed by the transgenic rodent, or genes encoding the antibodies.

34. The method of claim 32, further comprising:
(i) obtaining B cells from the transgenic rodent expressing antibodies specific for the target antigen;
(ii) immortalizing the B cells; and
(iii) isolating antibodies specific for the target antigen from the immortalized B cells.

35. The method of claim 34, further comprising cloning equine variable regions from the B cells specific for the particular antigen.

36. The method of claim 35, further comprising producing a therapeutic or diagnostic antibody using the equine variable regions cloned from the B cells.

37. A therapeutic or diagnostic antibody produced by the method of claim 32.


SEQUENCE LISTING (124 sequences)

SEQ ID NO: 1 - DNA, 353 bp, Artificial Sequence
  Description of Artificial Sequence Synthetic polynucleotide
atggactggagctggagcatcctcttcttggtggcagtggctgcaggtgtctcctccgaggtccagctggtgcagtctggggctgaggtgaagaagccaggggcatccgtgaaggtctcctgcaaggcttctggagacagcttcacttattactctatgagctgggtgcgacaggcccctggacaagggcttgagtggacgggatatatctatcctgaatatgatgctatgggctacccgcagaagttccagggcagagtcaccatgactgcggacaagtccacgagcacagtctacatggagctgagcagtctggcatctgaggacacagccgtgtattactgtgcaacaga

SEQ ID NO: 2 - DNA, 350 bp, Artificial Sequence
  Description of Artificial Sequence Synthetic polynucleotide
atgaatcacctgtggttcttcctctttctggtggccgctcctgcatgtgtcctgtcccaggtgcaactgaaggagtcaggacctggcctggtgaagccctcgcagaccctgtccctcacctgccctgtctctagattccctttaaccaaccatcatgtacactggacccaccaggctccaggaaaagggctggagtggcttggtgattcaaggagtggtgaaagcacatactacaacttaactctgaagtcccaactcagcatccccagtgatacttccaaaagccaaatttatttaacgctgaacaggctgagaggcgatgacatggccatgtactactgtgccagaga

SEQ ID NO: 3 - DNA, 356 bp, Artificial Sequence
  Description of Artificial Sequence Synthetic polynucleotide
atgagactcttgtgtcttctcctttgcctggtgatggctccccaaggagtcctgtcccaggtgaagctgcaggagtcgggcccaggactggtgaagccctcacagaccctctccctcacctgctctgtgtctggagtctccatcacaagcagtggtgactggtggagctggatccgccagcccccagggaaggggctggaatggatggggtacataagttatagtggtagcgcttactacaccacatccctcaagagccgactctccatctccagagacacgtccaaggaccagttctccctgcagctgagctccgtgaccacagaggacacggccgtttattactgtgcaagtga

SEQ ID NO: 4 - DNA, 350 bp, Artificial Sequence
  Description of Artificial Sequence Synthetic polynucleotide
atgaatcacctgtggttcttcctctttctggtggccgctcctacatgtgtcctgtcccaggtgcaactgaaggagtcgggacctggcctggtgaagccctcgcagaccctgtccctcacctgcactgtctctggattatctttgagcagtaatgctgtaggctgggtccgccaggctccaggaaaagggctggagtgggttggtgttatatatggtagtgaaagtacatactacaacccagccctgaagtcccgagccagcatcaccaaggacacctcaaagagccaagtttatctgacgctgaacagcctgacaggcgaagacacggccgtctattactgtgcaggatg

SEQ ID NO: 5 - DNA, 348 bp, Artificial Sequence
  Description of Artificial Sequence Synthetic polynucleotide
atgagtcacctgtggttcttcctctttctggtggccgctcctacatgtgtcctgtcccaggtgcaactgaaggagtcaggacctggcctggtgaagccctcgcagaccctgtccctcacctgcactgtctctggattatctttgagcagttatgctgtaggctgggtccgccaggctccaggaaaagggctggaatatgttggtgctatatatggtagtgcaagtgcaaactacaacccagccctgaagtcccgagccagcatcaccaaggacacctcaaagagccaagtttatctgacgctgaacagcctgacaggcgaggacacggccgtctattactgtgcgaga

SEQ ID NO: 6 - DNA, 353 bp, Artificial Sequence
  Description of Artificial Sequence Synthetic polynucleotide
atggactggagctggagcatcctcttcttggtggcagtggctgcaggtgtctcctccgaggtccagctggtgcagtctggggctgaggtgaggaagccaggggcatccgtgaaggtctcctgcaaggcttctggagacagcttcacttattactctatgagctgggtgcgacaggcccctggacaagggctcgactggatgggagggatcttgcctatagttgatgatacaagctacacgcagaagttccagggcagagtcaccatgactgcagacaagtccacgagcacagtctacatggagctgagcagtctgacatccgaggacacggccgtgtattactgtgcaaaaga

SEQ ID NO: 7 - DNA, 353 bp, Artificial Sequence
  Description of Artificial Sequence Synthetic polynucleotide
atgggctggagctggagaatcctcttcttggtggcagtagcttcaggtgtctcctccgagggtcagctggaacagtcggggccggagttgaagaagcctgggtcatcagtgaagatctcctgcaaggcttctggatacaccttcagtagctatgctgtgcactgggtgcgacaggccaatggaaaagggattgagtggatgggatctatctatgctgaatatgatgatacaagctacgcaccgaagttccagggcagagtcaccatgactgcggacaagtccacgagcacagtctacatggagctgagcagtctgacatctgaggacatggccgtgtattactgtgcaacaga

SEQ ID NO: 8 - DNA, 350 bp, Artificial Sequence
  Description of Artificial Sequence Synthetic polynucleotide
atggcccctctcctggtcatcttctgcctgctggctgctctccacggtgtcgaggctgaggaccctctcgtgcaatggggaggtggagtggtggtctcctcacagacactcagcctcacctgtgccgcctacaaacgcaaagtttcagaatattccctgtggtggattcgccttctcccagggaaggggttggagtgcgtaggtgtgatctgggctaagggggacactcagtgcagcccccacctgcagtctcgagtcagcatctccagggacgccaccaagaaccaagtgttcttacagctgagcagtgtgatgcctgaggattcaggcgtgtattactgtgctcaaga

SEQ ID NO: 9 - DNA, 356 bp, Artificial Sequence
  Description of Artificial Sequence Synthetic polynucleotide
atgagactcttgtgtcttctccttttcctggtgacggctccccaaggagtcctgtcccaggtgcagctgcaggagtcgggcccaggactggtgcagccctcacagaccctgtccctcacctgcactgtcactggaggctccatcacaagcagctattctagctggagctggttacgccagcctccagggaaggggctggagtacatggggtacatatattatgatggtagaacttactacaatccttccttcaagagccgcacctccatctccagagacacctccaggaaccagttctccctgcagctgagctccgtgaccaccgaggacgcggccgtgtattactgtgcaagaga

SEQ ID NO: 10 - DNA, 358 bp, Artificial Sequence
  Description of Artificial Sequence Synthetic polynucleotide
atggacacactgtatcccaccctcctgctgctgaccatcccttcctgggctctgtcccagatcagcctgcaggagtctggtcctgggctgctgaagcccacccagacccttacgctgacctgctccttctctgggttctcactgactacttctgatattggtgttggttggatgcgtcaaccccctgggaaggcactggagtggctcacctatgtttggtggactgatgaaaagcattacaacccatctctgaagagccggctcacaatctccaaggacacctccaaaaaccaggtgatgctgacaatgaccagtttggaccctccagacacagccacatattactgtgtaaagaggg

SEQ ID NO: 11 - DNA, 353 bp, Artificial Sequence
  Description of Artificial Sequence Synthetic polynucleotide
atgaggaggctgggtcttctccttttcctggtgacggctccccaaggtgtcctctcccaggtgcagctgcaggagtcaggaccaggccagacgaatccctcacagaccctgtccctcacatgcactgtcactggttactccatcaccagtggttatggctggaactggatccgccagccaccaaacaaagggctggagtggatggggagcataagctatagtggtagaactaactacagcccatccctcaggagccgcatcaccatctccagagacacttccaagaaccagttcttgctgcagctgagctcagtaaccactgaggacacggccgtgtattactgtgcgacaga

SEQ ID NO: 12 - DNA, 353 bp, Artificial Sequence
  Description of Artificial Sequence Synthetic polynucleotide
atgaggctgttgggtcttctcctttgtcttgtgacggcttaccagggtgtcctgtcccaggtgcagctgcaggagtcgggcccaggactggtgaagccctcacagaccctctccctcacctgcactgtcactggttactccatcaccagtggttactactggagctggatccgtcagcccccaggaaagaggctggagtggatgggctccatatattatagtggtagcacttactacagcccatccctcaagagccgcatcaccatctccacagacacgtcccagaaccagtcctccctgcagctgagctccgtgaccaccaaggacacagctgtgtattactgtgccagaga

SEQ ID NO: 13 - DNA, 31 bp, Artificial Sequence
  Description of Artificial Sequence Synthetic oligonucleotide
gtattattctcctggatagagtaattacaac

SEQ ID NO: 14 - DNA, 19 bp, Artificial Sequence
  Description of Artificial Sequence Synthetic oligonucleotide
ttactatggctggggtaac

SEQ ID NO: 15 - DNA, 32 bp, Artificial Sequence
  Description of Artificial Sequence Synthetic oligonucleotide
atgatgactatggtgatactttctactataca

SEQ ID NO: 16 - DNA, 31 bp, Artificial Sequence
  Description of Artificial Sequence Synthetic oligonucleotide
gtattactcttctgcatatgattgtatcaac

SEQ ID NO: 17 - DNA, 19 bp, Artificial Sequence
  Description of Artificial Sequence Synthetic oligonucleotide
ttactacaactataactac

SEQ ID NO: 18 - DNA, 29 bp, Artificial Sequence
  Description of Artificial Sequence Synthetic oligonucleotide
atggttactatagtaggagttgctatacc

SEQ ID NO: 19 - DNA, 44 bp, Artificial Sequence
  Description of Artificial Sequence Synthetic oligonucleotide
ggatttactgttctgggtgcagatgctcgttacaaccacagcaa

SEQ ID NO: 20 - DNA, 19 bp, Artificial Sequence
  Description of Artificial Sequence Synthetic oligonucleotide
taactacagatatagctcc

SEQ ID NO: 21 - DNA, 30 bp, Artificial Sequence
  Description of Artificial Sequence Synthetic oligonucleotide
atggttactatgctagtggttatgactaca

SEQ ID NO: 22 - DNA, 31 bp, Artificial Sequence
  Description of Artificial Sequence Synthetic oligonucleotide
gtattactcttctgcatatgcttgtatcaac

SEQ ID NO: 23 - DNA, 24 bp, Artificial Sequence
  Description of Artificial Sequence Synthetic oligonucleotide
taactacggttatggttatgctac

SEQ ID NO: 24 - DNA, 26 bp, Artificial Sequence
  Description of Artificial Sequence Synthetic oligonucleotide
actatagttatggtagttactatgcc

SEQ ID NO: 25 - DNA, 34 bp, Artificial Sequence
  Description of Artificial Sequence Synthetic oligonucleotide
gtatgactgtactggtcatggatgtgtctacatc

SEQ ID NO: 26 - DNA, 19 bp, Artificial Sequence
  Description of Artificial Sequence Synthetic oligonucleotide
taactactatggtagcaac

SEQ ID NO: 27 - DNA, 38 bp, Artificial Sequence
  Description of Artificial Sequence Synthetic oligonucleotide
atggttactatggtagttactacagtagttactatgcc

SEQ ID NO: 28 - DNA, 31 bp, Artificial Sequence
  Description of Artificial Sequence Synthetic oligonucleotide
gtattactattctggatataattattacaac

SEQ ID NO: 29 - DNA, 17 bp, Artificial Sequence
  Description of Artificial Sequence Synthetic oligonucleotide
gccactgatatagctcc

SEQ ID NO: 30 - DNA, 29 bp, Artificial Sequence
  Description of Artificial Sequence Synthetic oligonucleotide
atggttactatgctggtagttactatgcc

SEQ ID NO: 31 - DNA, 34 bp, Artificial Sequence
  Description of Artificial Sequence Synthetic oligonucleotide
gtgtgaatgtcctgggcatggatgttattacgac

SEQ ID NO: 32 - DNA, 19 bp, Artificial Sequence
  Description of Artificial Sequence Synthetic oligonucleotide
ttcctaccgatatagctcc

SEQ ID NO: 33 - DNA, 33 bp, Artificial Sequence
  Description of Artificial Sequence Synthetic oligonucleotide
acggttcctatgctggtagttacttatactaca

SEQ ID NO: 34 - DNA, 31 bp, Artificial Sequence
  Description of Artificial Sequence Synthetic oligonucleotide
gtattactattctgcatatgattattacaac

SEQ ID NO: 35 - DNA, 30 bp, Artificial Sequence
  Description of Artificial Sequence Synthetic oligonucleotide
atgattactatggtattagtgactcctaca

SEQ ID NO: 36 - DNA, 31 bp, Artificial Sequence
  Description of Artificial Sequence Synthetic oligonucleotide
gtattactcttttgaatatggttataacaac

SEQ ID NO: 37 - DNA, 25 bp, Artificial Sequence
  Description of Artificial Sequence Synthetic oligonucleotide
ctgctatagcagctatgcttactac

SEQ ID NO: 38 - DNA, 30 bp, Artificial Sequence
  Description of Artificial Sequence Synthetic oligonucleotide
actatggttatggtggtgcttactactaca

SEQ ID NO: 39 - DNA, 31 bp, Artificial Sequence
  Description of Artificial Sequence Synthetic oligonucleotide
gtattactattctgcatttcgttattacaac

SEQ ID NO: 40 - DNA, 25 bp, Artificial Sequence
  Description of Artificial Sequence Synthetic oligonucleotide
ctgctatagcagctatgcttactac

SEQ ID NO: 41 - DNA, 32 bp, Artificial Sequence
  Description of Artificial Sequence Synthetic oligonucleotide
acagttactatggtggtagttcctggtactcc

SEQ ID NO: 42 - DNA, 39 bp, Artificial Sequence
  Description of Artificial Sequence Synthetic oligonucleotide
gtattactattctggacatgattattacaacctcagcgt

SEQ ID NO: 43 - DNA, 22 bp, Artificial Sequence
  Description of Artificial Sequence Synthetic oligonucleotide
ttacgatgacggatactacaac

SEQ ID NO: 44 - DNA, 18 bp, Artificial Sequence
  Description of Artificial Sequence Synthetic oligonucleotide
ggtcctgggtacagctcc

SEQ ID NO: 45 - DNA, 21 bp, Artificial Sequence
  Description of Artificial Sequence Synthetic oligonucleotide
agatactccagtgctggttac

SEQ ID NO: 46 - DNA, 18 bp, Artificial Sequence
  Description of Artificial Sequence Synthetic oligonucleotide
ctacggtagcggttggcc

SEQ ID NO: 47 - DNA, 25 bp, Artificial Sequence
  Description of Artificial Sequence Synthetic oligonucleotide
taactatggctcctataattactac

SEQ ID NO: 48 - DNA, 31 bp, Artificial Sequence
  Description of Artificial Sequence Synthetic oligonucleotide
atgattattatggtgctattgactacataac

SEQ ID NO: 49 - DNA, 35 bp, Artificial Sequence
  Description of Artificial Sequence Synthetic oligonucleotide
tatgacaattctgtatatagctctgactacagcat

SEQ ID NO: 50 - DNA, 19 bp, Artificial Sequence
  Description of Artificial Sequence Synthetic oligonucleotide
ggagaagagttggagtaac

SEQ ID NO: 51 - DNA, 29 bp, Artificial Sequence
  Description of Artificial Sequence Synthetic oligonucleotide
acagttactggagtagtagttactatgcc

SEQ ID NO: 52 - DNA, 31 bp, Artificial Sequence
  Description of Artificial Sequence Synthetic oligonucleotide
gaataactatgctacatatgattatatcaac

SEQ ID NO: 53 - DNA, 18 bp, Artificial Sequence
  Description of Artificial Sequence Synthetic oligonucleotide
aactgctatggtaacaac

SEQ ID NO: 54 - DNA, 19 bp, Artificial Sequence
  Description of Artificial Sequence Synthetic oligonucleotide
ggtacttgggtacagctcc

SEQ ID NO: 55 - DNA, 21 bp, Artificial Sequence
  Description of Artificial Sequence Synthetic oligonucleotide
agatactccagtgttggttac

SEQ ID NO: 56 - DNA, 18 bp, Artificial Sequence
  Description of Artificial Sequence Synthetic oligonucleotide
atacggtagtggttggcc

SEQ ID NO: 57 - DNA, 54 bp, Artificial Sequence
  Description of Artificial Sequence Synthetic oligonucleotide
cttatgcttacttgcagcactggggccacggcaccctggtcaccgtctcctcag

SEQ ID NO: 58 - DNA, 53 bp, Artificial Sequence
  Description of Artificial Sequence Synthetic oligonucleotide
gtcctggcacctcgagcactgggaccacggcatcctggtcaccgtctcctcag

SEQ ID NO: 59 - DNA, 54 bp, Artificial Sequence
  Description of Artificial Sequence Synthetic oligonucleotide
gttatggctacgtggatcactggggccagggcaccctggtcaccgtctcctcag

SEQ ID NO: 60 - DNA, 48 bp, Artificial Sequence
  Description of Artificial Sequence Synthetic oligonucleotide
actattttggctactggggccagggcaccctggtcaccgtctcctcag

SEQ ID NO: 61 - DNA, 51 bp, Artificial Sequence
  Description of Artificial Sequence Synthetic oligonucleotide
acaacgagttggattactggggccagggcaccctggtcaccgtctcctcag

SEQ ID NO: 62 - DNA, 54 bp, Artificial Sequence
  Description of Artificial Sequence Synthetic oligonucleotide
attattatggtataaactactggggccagggcatcctggtcaccgtctcctcag

SEQ ID NO: 63 - DNA, 57 bp, Artificial Sequence
  Description of Artificial Sequence Synthetic oligonucleotide
attattataatgctatggacccctggggccagggcaccctggtcaccgtctcctcag

SEQ ID NO: 64 - DNA, 54 bp, Artificial Sequence
  Description of Artificial Sequence Synthetic oligonucleotide
attattatgatatagactactggggccagggcaccctggtcaccgtctcctcag

SEQ ID NO: 65 - DNA, 54 bp, Artificial Sequence; identical to SEQ ID NO: 64
  Description of Artificial Sequence Synthetic oligonucleotide

SEQ ID NO: 66 - DNA, 362 bp, Artificial Sequence
  Description of Artificial Sequence Synthetic polynucleotide
atgaggttctctgctcagctcctggggttgctaatactctgggtcccaggatccactggggacacagttttgacccagaccccactctctctgtctgtcatccctggagagtcggcctccatctcttgcaagtctagtcagagcctcctacatggtaatggaaacacctatttgcattggtacctgcagaagccaggccagtctcttcagcgcctgatctctatggtttccaatcgggcatctggggtcccagacaggttcagtggcagcgggtctgggacagatttcacccttataatcagcaagttggaagctgaggatgttggagtttattactgcatgcaagctacacaaagtccccc

SEQ ID NO: 67 - DNA, 362 bp, Artificial Sequence
  Description of Artificial Sequence Synthetic polynucleotide
atgaaattcgctagtcagctcctggggctactgatgctctggatcccaggatccagtgcggatgttgtgttgacccagactccactctccctgtctgtcgtccctggagagccggcctccatctcctgcaagtctagtcagagcctcaaatatagtgatgggaaaacctatttgtattggttcctacagaagccaggccagtctccaaagctcctgatctatttggtttccacccggtactctggggtctcagacaggttcagtggcagcggatcagaagcagatttcaccctgaaaatcagcagagtggagcctgaggatgttggagtctattactgctttcaagctctatatgcttctcc

SEQ ID NO: 68 - DNA, 362 bp, Artificial Sequence
  Description of Artificial Sequence Synthetic polynucleotide
atgaggctccctgctcagctcctggggctgctgatgctctggatcacaggatccagtggggatgttgtgatgacccagactccactctccctgtctgtcgtccctggagagccggcctccatctcctgcaagtctagtcagagcctcaaacatagtgatggaaaaacctatttgtattggttcctacagaagccaggccagtctccaaagtgcttgatctatttggtttccacccgggtctctggagtctcagacaggttcagtgggagcgggtcagaaacagatttcaccctgaaaatcagcagaggggagcctgaggacgttggagtctattactgtgtgcaagctctatatgcttctcc

SEQ ID NO: 69 - DNA, 347 bp, Artificial Sequence
  Description of Artificial Sequence Synthetic polynucleotide
atgggctcccaggctcagctcctcagcttcctgctcctctggatttttgataccagggcagaaataacagtcacacagtctccggaatccatgttagtgattccaggagacaaagtcatcatcacctgcaaagccagccaagacattggtgatgatgtgaactggtatcaatggaaaccaggagaagctcctaagctcattattaaagaagctactactctctggtctggggttccctctcggttcagtggcactgtgcatggagtagattttaccctgacaattgaggacgtaaaatctgaggatgctgcatattatttctgtctacaacatgatcgtatacctct

SEQ ID NO: 70 - DNA, 362 bp, Artificial Sequence
  Description of Artificial Sequence Synthetic polynucleotide
atgaggctccctgctcagctcctggggctgctgatgctctggatcccaggatccagtggggatgttgtgatgacccagactccactctccctgcctgtcgtccctggagagccggcctccatctcctgcaagtctagtcagagcctgctggatagtgatggaaaaacctatttgtattggtacctgcagaagccgggccagtctccaaagctcctgatctattcagtttccaaccgggactctgggatctcagacaggttcagtggcagcgggtcaggaacagatttcaccctgaaaatcagcagagtggagcctgaggatgttgaagtctattactgtgtgcaagctacacatgctcctcc

SEQ ID NO: 71 - DNA, 347 bp, Artificial Sequence
  Description of Artificial Sequence Synthetic polynucleotide
atgagggtccctgctcagctcctcagccttctgctgctctggctcccaggtgccaggtgtgagatccagatgacccagtctccagcctccctgtctgcatctctaggagacagagtcaccatcacttgccaggccactcagggcattaacacttggttagcctggtatcagcagaaaccagggaaagctcttaagttcctgatcagtaaggcaaccattttgcacactggcgtctcttcgaggttcagtggcagtggaacttggacagatttcactctcaccatcagcagcctggagcctgaagatgctgcaacttattactgtcagcagtataagagcagccctcc

SEQ ID NO: 72 - DNA, 362 bp, Artificial Sequence
  Description of Artificial Sequence Synthetic polynucleotide
atgaaattccttgctcagctcctggggctgctaatgctctggatcccaggatccagtggagatattgtgatgacccagactccactctccctgcctgtcgtccctggagagctggcctccaactcatgcaggtttagtcagagcctcctacatagtaatggaaacacctatttgcactggttcctgcagaagccaggccaatctccaaggcgtctgacctatagggtgtccaaccggaactctggggtcccagacaggttcattggcagcgggtcagggacagattttacacttaaaatcagcaaggtggaggctgaagatggtggagtttattattgctcccaaggtacacaaagtccccc

SEQ ID NO: 73 - DNA, 362 bp, Artificial Sequence
  Description of Artificial Sequence Synthetic polynucleotide
atgaggttccctgctcagctcctggggctactaatgctctggatcccaggatccagtggagatattgtgatgacccagactcccctctgcttggccgtcaccttgggagagccagtttccatctcctgcaggtctagtcagagcctcctccgtagtgatgactacacctatttggattggtacctgcagaaaccaggccagtctccacggctgctgatctatgaggtttccaagctggtctctggagtctcagacaggttcagtggcagtgggtcagggacagatttcacccttcaaatcagcagagtggaggctgaggatgttggagtttattactgcatgcaaggttcacaaagacctcc

SEQ ID NO: 74 - DNA, 347 bp, Artificial Sequence
  Description of Artificial Sequence Synthetic polynucleotide
atgatgtcactgacaaaggtgtttatgtctttgttgctctgggtctcaactgcctgtggggacatcgtgatgacccagtctccaggctccttggcagtgtctccaggacagagggtcaccattagctgcaaggccagtcagagtgttagcaactacttagactggtaccagcaaaaaccaggagaggctcctatgctgcttatctatgcggcatccagcagagcatctggggtccccgacagattcagtggcggtggatctgggacagatttcgctctcaccatcagcagcctccaggctgaagatgtggcagttttcactgtcagcagcattatactaatcctccc

SEQ ID NO: 75 - DNA, 347 bp, Artificial Sequence
  Description of Artificial Sequence Synthetic polynucleotide
atgatgtgggagacacaggtccttatgtccttattgctcggggtctcaggtaccttgggggacatcatgatgacccagtctccagactccttggcagtgtctctaggagagagggtcgacatgaagtgcacggccagtcagagtgtttaccactacttagcctggtaccagcaaaaaccaggacaggctcctaagcccctcatctactcagcatctaccagaccatctgggatccctgaccgattcagtggcagtggatctgggacagatttcactctcaccatcagcaacttccaagctgaagatgcggcagtttattgctgtgagcagtattatggtaatcctcc

SEQ ID NO: 76 - DNA, 347 bp, Artificial Sequence
  Description of Artificial Sequence Synthetic polynucleotide
atgatgtcacagacacaggtcctcttgtcggtgtttctctgggtctcaggtgcctgtggggacctcgtgatgacgcagtctccaggctccttggcagcgtctctaggacagagagtcgagatgaagtgcaaggccagtcagagtgttagcagctacttagactggtaccagcagaaaccaggacaggctcctaagcagctcatctatgctgcatccagcagagcgtctggggtccccgaccgattcagtggcagtggatctgggacagatttcactatcaccatcagcagcctccaggctgaagatctggccatttattactgtcagcagtataatagtgctcctcc

SEQ ID NO: 77 - DNA, 347 bp, Artificial Sequence; identical to SEQ ID NO: 76
  Description of Artificial Sequence Synthetic polynucleotide

SEQ ID NO: 78 - DNA, 347 bp, Artificial Sequence
  Description of Artificial Sequence Synthetic polynucleotide
atgatgttgcagacacaggtccttataaccttgttgctctgggtctcaggtgcctgtggggacatcgtgatgacccagtctccagactccttgtctgtgtctgcaggacaaagggtcgacatgaagtgcagggccagtcagagtgttagcaatgagttatcctggtaccagcaaaaaccaggacaggctcctaagctgctgatctatgcagcatccaacagagcatctgtggtccctgaccgattcagtggcggtggatctgggacagatttcactctcaccatcagtagcctccaggctgaagatgtggccgtttattactgtctgcagcattataataatcctcc

SEQ ID NO: 79 - DNA, 347 bp, Artificial Sequence
  Description of Artificial Sequence Synthetic polynucleotide
atgatgtcgctgacaaaggtccttatatctgtgttgctctgggtctcaggtgcctgtggggacatcgtgttgacccagtctccagagtccttggcagtgtctctaggacagagggtcgagatgaagtgcaaggccagtcagagtgctagcagcaacttggactggcaccagcacaaaccaggacaggctcctaagcagctcatctacagagcatccagcagagcgtctggggtccctgaccgattcagtggcagtggatctgggacagatttcactctcaccatcagcagcctccaggctgaagatgtggccgtttattactgtcagcagtataatagtgctcctcc

SEQ ID NO: 80 - DNA, 347 bp, Artificial Sequence
  Description of Artificial Sequence Synthetic polynucleotide
atgatgtcatggactcagatccttatgtccttgttgctctgggtctcaggtgcctgtggggacatcgtgatgacccagtctccagactccttggcagtgtctctaggacagagagtcgagatgaagtgcaaggccagtcagagtgttagcaactacttagactggtaccagcaaaaaccagtaaaggctcctaagctgctcatctatgcagcatccagcagagcatctggggtccccgaccgattcagtggcagtggatctgggacagatttcactctcaccatcagcagcctccaggctgaagatgtggcagtttactcctgtcagcagcgttatagttctcctcc

SEQ ID NO: 81 - DNA, 347 bp, Artificial Sequence
  Description of Artificial Sequence Synthetic polynucleotide
atgatgtcgctgacacagttccttatatctgtgttgctctgggtctcaggtgcctgtggggacatcgtgatgacgcagtctccagactccttggcagtgtctctaggacagagagtcgagatgaagtgcaaggccagtcagagtgttagcagcagcttggactggcaccagcacaaaccaggacaggctcctaagctgctcatctacagagcatccagcagagcgtctggggtccctgaccgattcagtggcagtggatctgggacagatttcactctcaccatcagcagcctccaggttgaagatgtggcagtttattactgtatgcagtctaatactgctcctcc

SEQ ID NO: 82 - DNA, 347 bp, Artificial Sequence
  Description of Artificial Sequence Synthetic polynucleotide
atgctgacgcagacgcaggtccttatatctgtgttgctctgggtctcaggagcctgtggggacgtcatgatgacccagtctccagactccttggcagcgtctctaggacagagagtcgagatgaagtgcaaggccagtcagagtgttagcagctacttagcttggtaccagcacaaaccaggacaggctcctaagcggctcatctatgctgcatccagcagagcatctggggtccctgaccgattcagtggcagtggatctgggatggatttcactctcaccatcagcagcctccaggctgaagatgtggccatttattactgtatgcagcattataataatcctcc

SEQ ID NO: 83 - DNA, 38 bp, Artificial Sequence
  Description of Artificial Sequence Synthetic oligonucleotide
gtggacgttcggtgccgggaccaagctggaaatcaaac

SEQ ID NO: 84 - DNA, 39 bp, Artificial Sequence
  Description of Artificial Sequence Synthetic oligonucleotide
tatacacgtttggccaagggaccaagctggagatcaaaa

SEQ ID NO: 85 - DNA, 38 bp, Artificial Sequence
  Description of Artificial Sequence Synthetic oligonucleotide
gttcactttcggccaagggaccaaactggagatcaaac

SEQ ID NO: 86 - DNA, 38 bp, Artificial Sequence
  Description of Artificial Sequence Synthetic oligonucleotide
gcttacgttcggccaggggaccaagctggagatcaaac

SEQ ID NO: 87 - DNA, 353 bp, Artificial Sequence
  Description of Artificial Sequence Synthetic polynucleotide
atggcctggtccccgctcctcctcaccctcatcgctctctgcacagtatcctgggcccagtctgtgctgactcagccggcctcagtgtctgggaacctgggccagagggtcaccatctcctgcactgggagcagctccaacaccagggataattatgtgaactggtaccagcagctcccaggaaccgcccccaaactcatcatctatgaaaatagcaaaagaccctccgggaccccagatcgaatctctggctccaagtctggaaacccggcctccctgaccatcactgggctccaggctgaggatgaggctgattattactgccagtcctatgatgacaacctgaatgctcg

SEQ ID NO: 88 - DNA, 356 bp, Artificial Sequence
  Description of Artificial Sequence Synthetic polynucleotide
atggcctggtcccctctcctcctcaccctcatcgctctctgcacagtcactactatcactgctgtaataggaaccacagtaataatcggcctcgtcctcagcctgaagcccagagatggtcagggtggctgtgttgccagacttggagccagagaatcgatctgggacccctgaggctcgtttgttattaccatagatgagggttttgggggctgttcctgggatctgttggtaccagcccacagcactataactatacccgatgttggagctgcttccagagcaggagatggtgactgtctggcccagggtcccagacactgaggcgggctgggtcagagactgggcccaggatc

SEQ ID NO: 89 - DNA, 356 bp, Artificial Sequence
  Description of Artificial Sequence Synthetic polynucleotide
atggcctggtcccctctcctcctcaccctcatcgctctctgcacaggatcctgggcccagtctctgactcagcccgcctcagtgtctgggaccctgggccagacagtcaccatctcctgctctggaagcagctccaacatcgggtatagttatagtgctgtgggctggtaccaacagatcccaggaacagcccccaaaaccctcatctatggtaataacaaacgagcctcaggggtcccagatcgattctctggctccaagtctggcaacacagccaccctgaccatctctgggcttcaggctgaggacgaggccgattattactgtggttcctattacagcagtgatagtagtga

SEQ ID NO: 90 - DNA, 347 bp, Artificial Sequence
  Description of Artificial Sequence Synthetic polynucleotide
atggcctggtgccctctcctcctcaccctcatcgctctctgcacaggatcctgggcccagtctgtgactcagcccgcctcagtgtctgggaccctgggccagacagtcaccatcacctgcactggaagcagctccaacatagttgcttatgtgggctggtaccaacagatcccaggaacagcccccaaaaccctcatctacgctaataacaaacgagcctcaggggtcccagatcgattctctggctccaagtctggcagcacagccaccctgaccatcactgggctccaggctgaggacgaggccgattattactgtggtacctctagcagcagtggtagtagtga

SEQ ID NO: 91 - DNA, 382 bp, Artificial Sequence
  Description of Artificial Sequence Synthetic polynucleotide
atgtcctgtactcctctcctcctcgtgctcctctctcactgcacaggttccctctcccagcctgtgctgacccagcctcccttcttctctgcatctcctggagcatcagccagactcacctgcaccctgagcagtgacatcagtgttgacagctctctcatattctggtgccagcagaagccagggagccctccccggtatctcctgagtttctactcagactcagttaagcaccagggctccggggtccccagccgcttctctggatccagagacacctcggccaatgcagggcttctgctcatctctgggctcaaggctgaggacgaggctgactattactgtgctacagctgatagcagtgggagcagctctggttact

SEQ ID NO: 92 - DNA, 350 bp, Artificial Sequence
  Description of Artificial Sequence Synthetic polynucleotide
atggcctggtcccctctcctcctcaccctcatcgctctctgcacaggatcccgggcccagtctgtgacgcagcccgcctcagtgtctgggaccctgggccagacagtcaccatctcctgctctggaagcagctccaacatcgggagtggtcatgtgtcctggtaccaacagatcccaggaacagcccccaaacgcctcatctattcttccgctagcagggcttccagggtccccgaccgattctctggctccaggtctggcaacacagccaccctgaccatctctgggctccagggtgaggacgaggccgattattactgtggtacattgtacagcagttggagtagtga

SEQ ID NO: 93 - DNA, 347 bp, Artificial Sequence
  Description of Artificial Sequence Synthetic polynucleotide
atggcctggtcccctctcctcctcaccctcatcgctctctgcacaggatcctgggcccagtctctgactcagcccgcctcagtgtctgggaccctgggccagacagtcaccatctcctgctctggaagcagctccagcataggttcttatatgggctggtaccaacagatcccagggacagcccccaaaaccctcatctatgctactaacaaacgagcctcaggggtcccagatcgattctctggctccaagtctggcaacacagccaccctgaccatctctgggcttcaggctgaggacgaggccgattattactgtggttcctattacagcagtgatagtagtga

SEQ ID NO: 94 - DNA, 353 bp, Artificial Sequence
  Description of Artificial Sequence Synthetic polynucleotide
atggcctggacagtgcttcttctctggctcctcacttacagctcaggggcagattctcaggctgtggtgatccaggacccatcattctctgtgtccctaggggggacggtcatactgacctgtggccttagaactgggtcagtctctaccagtaactatcctagatggtaccagcagacaccaggcaaggctccccgtacactcacctacagcacaaacaaccgcccctctgggatccctgaacgcttctctggatccatctcaggaaacaaagccgccctcaccatcacgggggcccagcccgaggacgaggccgactattactgtgatctgtatgtggatcgtggtgtttc

SEQ ID NO: 95 - DNA, 353 bp, Artificial Sequence
  Description of Artificial Sequence Synthetic polynucleotide
atggcctggatggtgcttcttctcgggctcctttcttacagctcaggggcggattctcagtctgtggtgacccaggagccatcactctcagtgtcttcaggagggacagtcacactcacctgtggccttaactctgggtcagtctcttccagtaaccaccccagctggcaccagcaaaccccaggccaggctccccgcacacttatctactacacaaacacccgtgcctctggagtccctaatctcttctctggatccatctccgggaacagagccaccctcaccatcacgggggcccagcgtgaggacgaggccgactattactgcgctctgtatacgggtagttacactga

SEQ ID NO: 96 - DNA, 347 bp, Artificial Sequence
  Description of Artificial Sequence Synthetic polynucleotide
atggcctggtcccctctcctcctcaccctcatcgctctctgcacaggatcctgggcccagtctgtgactcagcccgcctcagtgtctgggaccctgggccagacagtcaccatctcctgcactggaagcatctccaacataggtgtttatgtggactggtaccaacagatcccaggaacagcccccaaaaccatcatctatgctactaacaaacaaccctcaggggtcccagatcgattctctggctccaagtctggcaacacagccaccctgaccatcactgggctccaggctgaggacgaggctgattattactgtggtatctatgacagcagcctgagtagtga

SEQ ID NO: 97 - DNA, 37 bp, Artificial Sequence
  Description of Artificial Sequence Synthetic oligonucleotide
tgcgatggtcaggtgggtgcctccgccgaatgcacca

SEQ ID NO: 98 - DNA, 37 bp, Artificial Sequence; identical to SEQ ID NO: 97
  Description of Artificial Sequence Synthetic oligonucleotide

SEQ ID NO: 99 - DNA, 37 bp, Artificial Sequence; identical to SEQ ID NO: 97
  Description of Artificial Sequence Synthetic oligonucleotide

SEQ ID NO: 100 - DNA, 39 bp, Artificial Sequence
  Description of Artificial Sequence Synthetic oligonucleotide
gcgatgggtcaggtgggtgcctccgccgaatacagcaca

SEQ ID NO: 101 - DNA, 36 bp, Artificial Sequence
  Description of Artificial Sequence Synthetic oligonucleotide
aggactatcagctgggtccctcagctgagcacagga

SEQ ID NO: 102 - DNA, 38 bp, Artificial Sequence
  Description of Artificial Sequence Synthetic oligonucleotide
ctaggacggtcagatgggtacctccagtgaactatgaa

SEQ ID NO: 103 - DNA, 38 bp, Artificial Sequence; identical to SEQ ID NO: 102
  Description of Artificial Sequence Synthetic oligonucleotide

SEQ ID NO: 104 - DNA, 347 bp, Artificial Sequence
  Description of Artificial Sequence Synthetic polynucleotide
atggcctggacccctctcctgctcctcctcctcactctctgcacaggctctgtggcttcttctatgctgactcagccacttaccttgtccgtggcctttggaagcacagtcactatcacatgccagggagagctcctagacagttattatgctgagtggtaccagcagaagccagaccaggctcccgtgctggtcatatattatggaagcaaacgtccttcggggatttctacccgattctctggctcctactcaagcaagatggccaccctgaccctcagtggggccttggccgaggatgaggctgactattactgtcaggtgtgggacagcagtggtaaccagcc

SEQ ID NO: 105 - DNA, 342 bp, Artificial Sequence
  Description of Artificial Sequence Synthetic polynucleotide
atggcctggacccttctcctgcttcccctcctcactctctgcacaggttctgtgaccacctatgacttgacgcaaccacactcaacttcggtggccctaggacagacagcgacaatcacctgctctggagataatctcgaggatgaatatgcttactggtaccagcagaagacaggccagtcccctgccctggtcatttataaggatagtgagcacccctcagggatccctgaccggttctctggctcaaactcaggaaacacagccacgctgaccatcagaggggccaagacagaggacaaggctgactattactgccaatcgtggagcagtgctaatgct

SEQ ID NO: 106 - DNA, 348 bp, Artificial Sequence
  Description of Artificial Sequence Synthetic polynucleotide
atggcctggacccctctcttgtttgccttcctcactctctgcacaggtcctgtagtctcttctgaggtgactcagccaactgcggtgtccgtggccttgggacagacagcctccatcacctgccagggaagcgactttgaaaattattatgctagctggtaccagcagaagccaggccaggccccagtgctggtcatcaatgctaataatgagcggccctcagggatccctgaacgattctctgggtccagttcaggagagacagctacgctgaccatcagtggagcccacgctgaggacgaggccgactattactgtctggcaacagatgcttatgttgctgaagct

SEQ ID NO: 107 - DNA, 348 bp, Artificial Sequence
  Description of Artificial Sequence Synthetic polynucleotide
ctgtgcagagagtgaggaaggctaacaagagaggggtccaggccatagcttcataatcagaagcatctgctgccagacagtaatagtcagcctcgtcctcagcctgggccccgctgatggtcagcgtggctgtgtctcctgagctggagccagagaatcgttcagggatccctgagggccgctcattactagcatcgatgaccagcacaggggcctggcctggcttctgctggtaccagctaccaacaaaactttcaaagtcgcctcccttgcaggtgatggtggccgtctgtcccaaggccacagacactgaagatggctgagtcagcttagaagaggccacgggac

SEQ ID NO: 108 - DNA, 650 bp, Artificial Sequence
  Description of Artificial Sequence Synthetic polynucleotide
atggcctggacccctctcttgttagccttcctcactctctgcacaggtcctatggcctcttcggaggtgactcagccatctgcggtgtctgtggccttgggacagacagccaccctcacctgccagggagactactatgaaagatatattgtcaactggtaccagcagaagccaggccaggcacctgtgctggtcatctatgctaatagtgagcggccctcaggaatccctgaacgattctctggctccagctcattaggcacatccacgctgaccatcagcggggcccaggctgaggatgaggctgactattactgtcagccagcagatgctcatcgttctgaatctgtcctatggcctcttcggaggtgactcagccatctgcggtgtctgtggccttgggacagacagccaccctcacctgccagggagactactatgaaagatatattgtcaactggtaccagcagaagccaggccaggcacctgtgctggtcatctatgctaatagtgagcggccctcaggaatccctgaacgattctctggctccagctcattaggcacatccacgctgaccatcagcggggcccaggctgaggatgaggctgactattactgtcagccagcagatgctcatcgttctgaatct

SEQ ID NO: 109 - DNA, 348 bp, Artificial Sequence
  Description of Artificial Sequence Synthetic polynucleotide
atggcctggacccctctcttgttagccttcctctctctctgcacaggtcctgttgtctcttctgcagtgactcagccatctgaggtgtccgtggccttgggacagagagccaccctcacctgccagggaagcaactttgaatttttttctcctagctggtaccagcagaagccaggccaggcccctgtactgctcatcaatattaataatgagcgccactcagggatccctgaacgattctccggctccagctcaggagacacgtccacactgaccatcagtggggcccaggctgaggacgaggctgactattactgtctggcagtagatgctcttagttctgaaact

SEQ ID NO: 110 - DNA, 356 bp, Artificial Sequence
  Description of Artificial Sequence Synthetic polynucleotide
atggcctggacacttctccttctccctctcctcactctctgcacaggttctgtggccccttctgagctgactcagttaactgtggtgtctgtggccttggcacagacagccagggtcacctgccagggagagagaccaaaaagtgtctatgctggctggtaccagcagaagccaggccaggcccctgtatgggtcatctatagtaaaaacaattggaccacaggcacacctgaacaattctctgcctctgactcaggggacacagccaccctgaccatcagtggggtccaggttgagggcgagactgactattactgtggggtaagtgttggaagtgggagcagctggcagtcact

SEQ ID NO: 111 - DNA, 342 bp, Artificial Sequence
  Description of Artificial Sequence Synthetic polynucleotide
atggcctggacccctctcctgctccctctcctcactctctgcacaggttctgtgtcctcttctgagcttactcagtctactgcagtgtcattttccttgggacagacagccaccatcacctgccagggagaaaccctaagaagccactatgctagctggtaccagaagaatccaggacaggcccctgtattggtaatatatggtaataacaaccggccctcagggatccctgcccgattttccagctcctactcagaggacacaggcaccctgaccatcagtggggtccagatagaggatgaggctgactattactgccaatcattgggcagtgattatgct

SEQ ID NO: 112 - DNA, 354 bp, Artificial Sequence
  Description of Artificial Sequence Synthetic polynucleotide
atggcctgggctctgttcctcatcaccctcctcactcagggcacagggtcctggggccagtctgccctggttcagccttcttcggtgtccgtggctctaggacagtcggtcaccatctcctgtgctggaagcagcagtgacattgggtattataactctatttcctggtaccaacagcacccaggcacaaccccaaagctgctgatttactataccaataagaagcactcagggatccctgatcgcttctctggctccaagtctgggaacacggcctccctgaccatctctgggctccaggctgaggatgaggctgagtattactgttgctcatatgcaggcagtggcaattta

SEQ ID NO: 113 - DNA, 354 bp, Artificial Sequence
  Description of Artificial Sequence Synthetic polynucleotide
atggcctggactctgctccttctcaccctcctcactcagggtacagggtcctgggcccagtctgccctgactcagcctgcgtcagtgtccggggctctaggacagtcggtcaccatcacctgtgctggaagcagcagtgacattgggggttataatgctgtcagctggttacaacagcacccgggcacagcccccaaagttctgatttatagtgtgaatactcgggcctcagggatccctgatcgcttctctggctccaagtctggcaacacggcctccctgaccatctctgggctccaggttgaggacgaggctgattattactgttactcgcttgtgagtggttacactttc

SEQ ID NO: 114 - DNA, 355 bp, Artificial Sequence
  Description of Artificial Sequence Synthetic polynucleotide
atggcctgggctctgctcctcatcagcctcttcactcagggcacagggtcctgggcgcagtctgccctgactcagcctgcgtcagtgtccgggactctgggacagtcggtcaccatctcctgtgctggaagcagcagcaacattgggagttataactatgtttcctggtaccaacagcacccgggcacagcccccaaactcctcatttatagtgccagttctcgagcctcagggatccctgatcgcttctctggctccaagtctgggaacacggcctctctgaccatctcggggctccaggctgaggacgaggccgattattactgtagctcatatatcaatgctgatccttatc

SEQ ID NO: 115 - DNA, 345 bp, Artificial Sequence
  Description of Artificial Sequence Synthetic polynucleotide
atggcctgggctctgctcctcatcaccctcctcactcagggcacaggtaattgtaactgccaacatacgcgtgacagtaataatcagcctcgtcctcagcctggagcccagagatggtcagggacatcgtgttgccagacgtggagccggagaagcgatcagggatccctgaggcccgattattcccattataaatgaggagtttgggggctgtgcctgggtgctgttggtaccaggaaatatatttataagatcccgtgcttccagcacaggtgatggtgaccgactgtcccagagtcccggagactgacgcaggctgagtcagggcagactgcgcccaggacc

SEQ ID NO: 116 - DNA, 354 bp, Artificial Sequence
  Description of Artificial Sequence Synthetic polynucleotide
atggcctgggtgccactcctgctcacacttctggctcactgcacagggtccacttcacaggatgtggtgattcaggaatcttcactgatcacaactcctgggggaacagtcacactcacctgtggctcaagtgctggggctgtcacctccaataattatgccaactgggtccaagagaagccctatcagggacgccagggtctaataggtggtactagcaacagggtctcagggggtccctgcccgattctctggctccctgcgcttgggaacaaggccgccctcactatcatgggggcccagccagaggacgaggacgagtgttactgtgctctgtggttcagcaaccatttc

SEQ ID NO: 117 - DNA, 356 bp, Artificial Sequence
  Description of Artificial Sequence Synthetic polynucleotide
atggcctggtcccctctcctcctcaccctcatcgctctctgcacaggatcctgggcccagtctctgactcagcccgcctcagtgtctgggaccctgggccagacagtcaccatctcctgctctggaagcagctccaacatcgggtatagttatagtgctgtgggctggtaccaacagatcccaggaacagcccccaaaaccctcatctatggtaataacaaacgagcctcaggggtcccagatcgattctctggctccaagtctggcaacacagccaccctgaccatctctggggtccaggctgaggacgaggccgattattactgctcagcaggagacagcagtggtagtagtga

SEQ ID NO: 118 - DNA, 382 bp, Artificial Sequence
  Description of Artificial Sequence Synthetic polynucleotide
atggcctggactcctctcatcctcatgctcctgtctcactgcacaggttccctctcccagcctgtgctgacccagccaccctccctctctgcatctcctggaacatcagccagactcacctgcgccctgagcagtgatgtcagtgttagcagctctctcatattctggtaccagcagaagccagggagccctccggggtatcttctgagtttctactcagactcagttaagcaccagggctccggggtccccagccacttctctggatccaaagacacctcgtccaatgcagggcttctgctcatctctgggctcgaggctgaggacgaggctgactattactgtgctacagctgatagcagtgggatcagctctggttact

SEQ ID NO: 119 - DNA, 356 bp, Artificial Sequence
  Description of Artificial Sequence Synthetic polynucleotide
atggcctggtcccctctcctcctcaccctcatcgctctctgcacaggatcctgggcccagtctgtgactcagcccgcctcagtgtctgggaccctgggccagacagtcaccatctcctgctctggaagcagctccaacatcgggtatagttatagttatgtgggctggttccaacagatcccaggaacagcccccaaaaccctcatctatggtaataacaaacgagcctcaggggtcccagatcgattctctggctccaagtctggcaacacagccaccctgaccatctctggggtccaggctgaggacgaggccgattattactgtggttcctatgacagcagcagtagtagtga

SEQ ID NO: 120 - DNA, 348 bp, Artificial Sequence
  Description of Artificial Sequence Synthetic polynucleotide
atggcctggtcccctctcctcctcaccctcatcgctctctgcacagtcccgggcccagtctgtgactcagcccgcctcagtgtctgggaccctgggccagacagtcaccatctcctgctctggaagcagctccaacatcgggagtggtcatgtgtcctggtaccaacagatcccaggaacagcccccaaacgcctcatctattcttccactaatagggcttctggggtccccgaccgattctctggctccaggtctggcaacacagccaccctgaccatctctgggctccaggctgaggacgaggctgattattactgtggtacattgtacagcagttggagtaatga

SEQ ID NO: 121 - DNA, 336 bp, Artificial Sequence
  Description of Artificial Sequence Synthetic polynucleotide
atggcctggtcccctctcctcctcaccctcatcgctctctgcacagcagtctgtgactcagcccgcctcagtgtctgggaccctgggccagacagtcaccatctcctgcactggaagcatctccaacataggtgtttatgtggactggtaccaacagatcccaggaacagcccccaaaaccatcatctatgctactaacaaacaaccctcaggggtcccagatcgattctctggctccaagtctggcaacacagccaccctgaccatcactgggctccaggctgaggacgaggctgattattactgtggtatctatgacagcagcctgagtagtga

SEQ ID NO: 122 - DNA, 353 bp, Artificial Sequence; identical to SEQ ID NO: 95
  Description of Artificial Sequence Synthetic polynucleotide

SEQ ID NO: 123 - DNA, 100 bp, Mus musculus
atttctgtacctgatctatgtcaatatctgtaccatggctctagcagagatgaaatatgagacagtctgatgtcatgtggccatgcctggtccagacttg

SEQ ID NO: 124 - DNA, 100 bp, Mus musculus
gtcaatcagcagaaatccatcatacatgagacaaagttataatcaagaaatgttgcccataggaaacagaggatatctctagcactcagagactgagcac